(19) United States
(12) Patent Application Publication
(10) Pub. No.: US 2007/0258946 A1
(43) Pub. Date: Nov. 8, 2007

(54) Combination Therapy for Treating Hepatitis C Virus Infection

(76) Inventor: Lawrence M. Blatt, San Francisco (CA)

Correspondence Address: THELEN REID BROWN RAYSMAN & STEINER LLP, PO BOX 1510, NEW YORK, NY, 10150-1510, US

(21) Appl. No.: 10/584,169

(22) PCT Filed: Oct. 13, 2004
(86) PCT No.: PCT/US04/33959

§ 371 Date: Jul. 2, 2007

Related U.S. Application Data
(60) Provisional application No. 60/532,834, filed on Dec. 23, 2003.

(51) Int. Cl. A61K 38/21; A61P 31/14

(52) U.S. Cl. 424/85.5; 424/85.7

(57) ABSTRACT

The present invention provides methods of treating hepatitis C virus (HCV) infection; methods of reducing the incidence of complications associated with HCV and cirrhosis of the liver; and methods of reducing viral load, or reducing the time to viral clearance, or reducing morbidity or mortality in the clinical outcomes, in patients suffering from HCV infection. The methods generally involve administering to the individual i) a Type I interferon receptor agonist or a Type III interferon receptor agonist; ii) an immunomodulatory agent; and iii) an inhibitor of an HCV enzyme.

### FIELD OF THE INVENTION

This invention is in the field of viral infection, particularly hepatitis C viral infection.

### BACKGROUND OF THE INVENTION

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with combination therapy using pegylated IFN-A plus ribavirin, 40% to 50% of patients fail therapy, i.e., are nonresponders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1 960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

HCV is an enveloped positive strand RNA virus in the Flaviviridae family. The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins of the virus. In the case of HCV, the generation of mature nonstructural proteins (NS2, NS3, NS4, NS4A, NS4B, NS5A, and NS5B) is effected by two viral proteases. The first viral protease cleaves at the NS2-NS3 junction of the polyprotein. The second viral protease is serine protease contained within the N-terminal region of NS3 (herein referred to as “NS3 protease”). NS3 protease mediates all of the subsequent cleavage events at sites downstream relative to the position of NS3 in the polyprotein (i.e., sites located between the C-terminus of NS3 and the C-terminus of the polyprotein). NS3 protease exhibits activity both in cis, at the NS3-NS4 cleavage site, and in trans, for the remaining NS4A-NS4B, NS4B-NS5A, and NS5A-NS5B sites. The NS4A protein is believed to serve multiple functions, acting as a cofactor for the NS3 protease and possibly assisting in the membrane localization of NS3 and other viral replicase components.

Apparently, the formation of the complex between NS3 and NS4A is necessary for NS3-mediated processing events and enhances proteolytic efficiency at all sites recognized by NS3.

The NS3 protease also exhibits nucleoside triphosphatase and RNA helicase activities. NS5B is an RNA-dependent RNA polymerase involved in the replication of HCV RNA.

There is a need in the art for improved methods for treating viral infections, e.g. hepatitis C viral infection. The present invention addresses this need.

Literature

U.S. Pat. Nos. 6,642,204, 6,617,309; U.S. Pat. Nos. 6,524,570, 5,908,621, and 6,177,074; U.S. Pat. No. 5,382,657; METAVIR (1994) Hepatology 20:15-20; Brunt (2000) Hepatol. 31:241-246; Alpini (1997) J. Hepatol. 27:371-380; Baroni et al. (1996) Hepatol. 23:1189-1199; Czaja et al. (1989) Hepatol. 10:795-800; Grossman et al. (1998) J. Gastroenterol. Hepatol. 13:1058-1060; Rockey and Chung (1994) J. Invest. Med. 42:660-670; Sakaida et al. (1998) J. Hepatol. 28:471-479; Shi et al. (1997) Proc. Natl. Acad. Sci. USA 94:10663-10668; Baroni et al. (1999) Liver 19:212-219; Lortat-Jacob et al. (1997) J. Hepatol. 26:894-903; Llorent et al. (1996) J. Hepatol. 24:555-563; U.S. Pat. No. 5,082,659; European Patent Application EP 294,160; U.S. Pat. No. 4,806,347; Balish et al. (1992) J. Infect. Diseases 166:1401-1403; Katayama et al. (2001) J. Viral Hepatitis 8:180-185; U.S. Pat. No. 5,082,659; U.S. Pat. No. 5,190,751; U.S. Pat. No. 4,806,347; Wandl et al. (1992) Br. J. Haematol. 81:516-519; European Patent Application No. 294,160; Canadian Patent No. 1,321,348; European Patent Application No. 276,120; Wandl et al. (1992) Sem. Oncol. 19:88-94; Balish et al. (1992) J. Infectious Diseases 166:1401-1403; Van Dijk et al. (1994) Int. J. Cancer 56:262-268; Sundmacher et al. (1987) Current Eye Res. 6:273-276; U.S. Pat. Nos. 6,172,046; 6,245,740; 5,824,784; 5,372,808; 5,980,884; published international patent applications WO 96/21468; WO 96/11953; Torre et al. (2001) J. Med. Virol. 64:455-459; Belckering et al. (2001) J. Hepatol. 34:435-440; Zeuzem et al. (2001) Gastroenterol. 120:1438-1447; Zeuzem (1999) J. Hepatol. 31:61-64; Keeffe and Hollinger (1997) Hepatol. 26:101S-107S; Wills (1990) Clin. Pharmacokinet. 19:390-399; Heathcote et al. (2000) New Engl. J. Med. 343:1673-1680; Husa and Husova (2001) Bratisl. Lek. Listy 102:248-252; Glue et al. (2000) Clin. Pharmacol. 68:556-567; Bailon et al. (2001) Bioconj. Chem. 12:195-202; and Neumann et al. (2001) Science 282:103; Zalipslcy (1995) Adv. Drug Delivery Reviews S. 16, 157-182; Manm et al. (2001) Lancet 358:958-965; Zeuzem et al. (2000) New Engl. J. Med. 343:1666-1672; U.S. Pat. Nos. 5,985,265; 5,908,121; 6,177,074; 5,985,263; 5,711,944; 5,382,657; and 5,908,121; Osborn et al. (2002) J. Pharmacol. Exp. Therap. 303:540-548; Sheppard et al. (2003) Nat. Inmmunol. 4:63-68; Chang et al. (1999) Nat. Biotechnol. 17:793-797; Adolf (1995) Multiple Sclerosis 1 Suppl. 1:S44-S47.

### SUMMARY OF THE INVENTION

The present invention provides methods of treating hepatitis C virus (HCV) infection; methods of reducing the incidence of complications associated with HCV and cirrhosis of the liver; and methods of reducing viral load, or reducing the time to viral clearance, or reducing morbidity or mortality in the clinical outcomes, in patients suffering from HCV infection. The methods generally involve administering to the individual i) a Type I interferon receptor agonist or a Type III interferon receptor agonist; ii) an immunomodulatory agent; and iii) an inhibitor of an HCV enzyme.

### FEATURES OF THE INVENTION

The invention features a method of treating an HCV infection, generally involving administering to an individual (i) a Type I or a Type III interferon receptor agonist (ii) an immunomodulatory agent and (iii) an HCV enzyme inhibitor concurrently, in an amount effective to achieve a sustained viral response. In many embodiments, the Type I interferon receptor agonist is IFN-α. In some embodiments, the IFN-α is INFERGEN® consensus IFN-α. In some embodiments, the IFN-α is monoPEG(30 kD, linear)-ylated consensus IFN-α. In some embodiments, the IFN-α is PEG-INTRONαPEGylated IFN-α2b. In some embodiments, the IFN-α is PEGASYS®PEGylated IFN-α2a. In many embodiments, the immunomodulatory agent is selected from i) a Type II interferon receptor agonist; ii) a TNF antagonist; iii) pirfenidone or a pirfenidone analog; and iv) thymosin-α. In many embodiments, the subject method further provides administering to the individual an effective amount of a nucleoside analog, such as isatoribine, ribavirin, levovirin and viramidine.

In carrying out the methods of combination therapy for treating a hepatitis C viral infection in an individual as described above, (i) a Type I or Type III interferon receptor agonist (ii) an inmunomodulatory agent and (iii) an HCV enzyme inhibitor are administered to the individual. In some embodiments, two or more of the drugs are administered in the same formulation. In other embodiments, the Type I or III interferon receptor agonist, the immunomodulatory agent and the HCV enzyme inhibitor are administered in two or more separate formulations. When administered in separate formulations, a Type I or Type III interferon receptor agonist, an imununomodulatory agent, and an HCV enzyme inhibitor can be administered substantially simultaneously, or can be administered within about 24 hours of one another. In many embodiments, a Type I or Type III interferon receptor agonist, an immunomodulatory agent, and an HCV enzyme inhibitor are administered subcutaneously in multiple doses. Optionally, the Type I or Type III interferon receptor agonist, immunomodulatory agent, and/or HCV enzyme inhibitor is/are administered to the individual by a controlled drug delivery device. Optionally, the Type I or Type III interferon receptor agonist, immunomodulatory agent, and/or HCV enzyme inhibitor is/are administered to the individual substantially continuously or continuously by a controlled drug delivery device. Optionally, the controlled drug delivery device is an implantable infusion pump and the infusion pump delivers the Type I or Type III interferon receptor agonist, immunomodulatory agent, and/or HCV enzyme inhibitor to the individual by subcutaneous infusion.

In many embodiments, any of the above-described methods involve administering a Type I interferon receptor agonist that is a PEGylated IFN-α conjugate. In some embodiments, the PEGylated IFN-α conjugate is a monoPEGylated IFN-α. In other embodiments, the monoPEGylated IFN-α conjugate is an IFN-α polypeptide covalently linked to a single PEG moiety via a lysine residue or the N-terminal amino acid residue of the IFN-α polypeptide. In other embodiments, the monoPEGylated IFN-α conjugate is an IFN-α polypeptide covalently linked to a single PEG moiety via an amide bond between either the epsilon-amino group of a lysine residue or the alpha-amino group of the IFN-α polypeptide and an activated carboxyl group of the PEG moiety. In other embodiments, the monoPEGylated IFN-α conjugate is an IFN-α polypeptide covalently linked to a single, linear PEG moiety.

In other embodiments, the monoPEGylated IFN-α conjugate is an IFN-α polypeptide covalently linked to a single, linear 30 kD PEG moiety (“monoPEG(30 kD, linear)-ylated IFN-α”). In other embodiments, the monoPEGylated IFN-α conjugate is an IFN-α polypeptide covalently linked to a single, linear 30 kD PEG moiety via an amide bond between the epsilon-amino group of a lysine residue or the alpha-amino group of the IFN-α polypeptide and an activated carboxyl group of the PEG moiety. In other embodiments, the monoPEGylated IFN-α conjugate is an IFN-α polypeptide covalently linked to a single, linear 30 kD PEG via an amide bond between the epsilon-amino group of a lysine residue or the alpha-amino group of the IFN-α polypeptide and an activated propionyl group of the PEG moiety. In other embodiments, the monoPEGylated IFN-α conjugate is an IFN-α polypeptide covalently linked to a single, linear monomethoxy-PEG (mPEG). In other embodiments, the monoPEGylated IFN-α conjugate is the product of a condensation reaction between an IFN-α polypeptide and a linear, succinimidyl propionate ester-activated 30 kD mPEG. In any of the foregoing methods using a PEGylated IFN-α conjugate, the IFN-α polypeptide can be a consensus interferon (CIFN) polypeptide. In any of the foregoing methods using a PEGylated IFN-α conjugate, the IFN-α polypeptide can be a CIFN polypeptide that is interferon alfacon-1.

In one aspect, the present invention provides a combination therapy for treating an HCV infection in an individual, the method comprising administering IFN-α, IFN-γ, and an HCV enzyme inhibitor in combined effective amounts to achieve a sustained viral response. In some embodiments, the IFN-α is INFERGEN® consensus IFN-α. In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor.

In one aspect, the present invention provides a combination therapy for treating an HCV infection in an individual, the method comprising administering IFN-α, IFN-γ, ribavirin, and an HCV enzyme inhibitor in combined effective amounts to achieve a sustained viral response. In some embodiments, the IFN-α is INFERGEN® consensus IFN-α. In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor.

In one aspect, the present invention provides a combination therapy for treating an HCV infection in an individual, the method comprising administering IFN-α, IFN-γ, pirfenidone or a pirfenidone analog, and an HCV enzyme inhibitor in combined effective amounts to achieve a sustained viral response. In some embodiments, the IFN-α is INFERGEN® consensus IFN-α. In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor.

In one aspect, the present invention provides a combination therapy for treating an HCV infection in an individual, the method comprising administering IFN-α, pirfenidone or a pirfenidone analog, and an HCV enzyme inhibitor in combined effective amounts to achieve a sustained viral response. In some embodiments, the IFN-α is INFERGEN® consensus IFN-α. In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor.

In one aspect, the present invention provides a combination therapy for treating an HCV infection in an individual, the method comprising administering IFN-α, ribavirin, pirfenidone or a pirfenidone analog, and an HCV enzyme inhibitor in combined effective amounts to achieve a sustained viral response. In some embodiments, the IFN-α is INFERGEN® consensus IFN-α. In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor.

In one aspect, the present invention provides a combination therapy for treating an HCV infection in an individual, the method comprising administering IFN-α, an L-nucleoside, pirfenidone or a pirfenidone analog, and an HCV enzyme inhibitor in combined effective amounts to achieve a sustained viral response. In some embodiments, the IFN-α is INFERGEN® consensus IFN-α. In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor.

In one aspect, the present invention provides a combination therapy for treating an HCV infection in an individual, the method comprising administering IFN-α, IFN-γ, a TNF antagonist, and an HCV enzyme inhibitor in combined effective amounts to achieve a sustained viral response. In some embodiments, the IFN-α is INFERGEN® consensus IFN-α In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor. In some embodiments, the TNF antagonist is selected from HUMIRA, ENBREL, and REMICADE.

In one aspect, the present invention provides a combination therapy for treating an HCV infection in an individual, the method comprising administering IFN-α, a TNF antagonist, and an HCV enzyme inhibitor in combined effective amounts to achieve a sustained viral response. In some embodiments, the IFN-α is INFERGEN® consensus IFN-α In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor. In some embodiments, the TNF antagonist is selected from HUMIRA, ENBREL, and REMICADE.

In one aspect, the present invention provides a combination therapy for treating an HCV infection in an individual, the method comprising administering IFN-α, ribavirin, a TNF antagonist, and an HCV enzyme inhibitor in combined effective amounts to achieve a sustained viral response. In some embodiments, the IFN-α is INFERGEN® consensus IFN-α In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor. In some embodiments, the TNF antagonist is selected from HUMIRA, ENBREL, and REMICADE.

In one aspect, the present invention provides a combination therapy for treating an HCV infection in an individual, the method comprising administering IFN-α, an L-nucleoside, a TNF antagonist, and an HCV enzyme inhibitor in combined effective amounts to achieve a sustained viral response. In some embodiments, the IFN-α is INFERGEN® consensus IFN-α. In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor. In some embodiments, the TNF antagonist is selected from HUMIRA, ENBREL, and REMICADE.

In one aspect, the present invention provides a combination therapy for treating an HCV infection in an individual, the method comprising administering IFN-α, IFN-γ, an L-nucleoside, and an HCV enzyme inhibitor in combined effective amounts to achieve a sustained viral response. In some embodiments, the IFN-α is INFERGEN® consensus IFN-α In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor.

In one aspect, the present invention provides a combination therapy for treating an HCV infection in an individual, the method comprising administering IFN-α, IFN-γ, thymosin-α, and an HCV enzyme inhibitor in combined effective amounts to achieve a sustained viral response. In some embodiments, the IFN-α is INFERGEN® consensus IFN-α. In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor.

In one aspect, the present invention provides a combination therapy for treating an HCV infection in an individual, the method comprising administering IFN-α, thymosin-α, and an HCV enzyme inhibitor in combined effective amounts to achieve a sustained viral response. In some embodiments, the IFN-α is INFERGEN® consensus IFN-α. In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor.

In one aspect, the present invention provides a combination therapy for treating an HCV infection in an individual, the method comprising administering IFN-α, ribavirin, thymosin-α, and an HCV enzyme inhibitor in combined effective amounts to achieve a sustained viral response. In some embodiments, the IFN-α is INFERGEN® consensus IFN-α In some embodiments, the IFN-α is monoPEG(30 1kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor.

In one aspect, the present invention provides a combination therapy for treating an HCV infection in an individual, the method comprising administering IFN-α, an L-nucleoside, thymosin-α, and an HCV enzyme inhibitor in combined effective amounts to achieve a sustained viral response. In some embodiments, the IFN-α is INFERGEN® consensus IFN-α. In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor.

In one aspect, the present invention provides a combination therapy for treating an HCV infection in an individual, the method comprising administering IFN-α, IFN-γ, levovirin, and an HCV enzyme inhibitor in combined effective amounts to achieve a sustained viral response. In some embodiments, the IFN-α is INFERGEN® consensus IFN-α. In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor.

In one aspect, the present invention provides a combination therapy for treating an HCV infection in an individual, the method comprising administering IFN-α, IFN-γ, viramidine, and an HCV enzyme inhibitor in combined effective amounts to achieve a sustained viral response. In some embodiments, the IFN-α is INFERGEN® consensus IFN-α. In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor.

In one aspect, the present invention provides a combination therapy for treating an HCV infection in an individual, the method comprising administering IFN-α, IFN-γ, isatoribine, and an HCV enzyme inhibitor in combined effective amounts to achieve a sustained viral response. In some embodiments, the IFN-α is INFERGEN® consensus IFN-α. In some embodiments, the IFN-α is monoPEG(30 kD, linear)-ylated consensus IFN-α. In some embodiments, the IFN-α is PEG-INTRON®PEGylated IFN-α2b. In some embodiments, the IFN-α is PEGASYS®PEGylated IFN-α2a. In some embodiments, the HCV enzyme inhibitor is an NS3 protease inhibitor. In other embodiments, the HCV enzyme inhibitor is an NS5B RNA-dependent RNA polymerase inhibitor.

### Definitions

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, primates, including simians and humans.

The term “treatment failure patients” (or “treatment failures”) as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

The term “pharmacokinetic profile,” as used herein, refers to the profile of the curve defined by a patient's serum concentration of PEGylated IFN-α as a function of time, following the administration of PEGylated IFN-α to the patient. “Area under the curve,” or “AUC,” refers to the integrated area under the curve defined by a patient's serum concentration of PEGylated IFN-α as a function of time, following the administration of PEGylated IFN-α to the patient.

As used herein, the term “a Type I interferon receptor agonist” refers to any naturally occurring or non-naturally occurring ligand of human Type I interferon receptor, which binds to and causes signal transduction via the receptor. Type I interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

As used herein, the term “a Type III interferon receptor agonist” refers to any naturally occurring or non-naturally occurring ligand of human IL-28 receptor a (“IL-28R”), the amino acid sequence of which is described by Sheppard, et al., infra., that binds to and causes signal transduction via the receptor.

As used herein, the terms “immunomodulator” and “immunomodulatory agent” refer to any agent, other than (i) a Type I or Type III interferon receptor agonist and (ii) a nucleoside, that stimulates immune cell mediated destruction of virus-infected cells. The term “immunomodulatory agent” includes, but is not limited to, Type II interferon receptor agonists (including IFN-γ); TNF antagonists; pirfenidone and pirfenidone analogs; and thymosin-α (Zadaxin®; SciClone Pharmaceuticals); and the like.

As used herein, the term “nucleoside” refers to a compound composed of any pentose or modified pentose moiety attached to a specific position of a heterocycle or to the natural position of a purine (9-position) or pyrimidine (1-position) or to the equivalent position in an analog.

As used herein, the term “nucleotide” refers to a phosphate ester substituted on the 5′-position of a nucleoside.

As used herein, the term “heterocycle” refers to a monovalent saturated or unsaturated carbocyclic radical having at least one hetero atom, such as N, O, S, Se or P, within the ring, each available position of which can be optionally substituted, independently, with, e.g., hydroxyl, oxo, amino, imino, lower alkyl, bromo, chloro and/or cyano. Included within the term “heterocycle” are purines and pyrimidines.

As used herein, the term “purine” refers to nitrogenous bicyclic heterocycles.

As used herein, the term “pyrimidine” refers to nitrogenous monocyclic heterocycles.

As used herein, the term “L-nucleoside” refers to a nucleoside compound that has an L-ribose sugar moiety.

As used herein, the term “pirfenidone” refers to 5-methyl-1-phenyl-2-(1H)-pyridone. As used herein, the term “pirfenidone analog” refers to any compound of Formula I, IIA or IIB below. A “specific pirfenidone analog,” and all grammatical variants thereof, refers to, and is limited to, each and every pirfenidone analog shown in Table 1.

As used herein, the term “a Type II interferon receptor agonist” refers to any naturally-occurring or non-naturally-occurring ligand of a human Type II interferon receptor which binds to and causes signal transduction via the receptor. Type II interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

As used herein, the term “HCV enzyme inhibitor” refers to any agent that inhibits an enzymatic activity of an enzyme encoded by HCV. The term “HCV enzyme inhibitor” includes, but is not limited to, agents that inhibit HCV NS3 protease activity; agents that inhibit HCV NS3 helicase activity; and agents that inhibit HCV NS5B RNA-dependent RNA polymerase activity.

As used herein, the terms “HCV NS3 protease inhibitor” and “NS3 protease inhibitor” refer to any agent that inhibits the protease activity of HCV NS3/NS4A complex.

The term “hepatitis virus infection” refers to infection with one or more of hepatitis A, B, C, D, or E virus, with blood-borne hepatitis viral infection being of particular interest, particularly hepatitis C virus infection.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

As used herein, the term “hepatic fibrosis,” used interchangeably herein with “liver fibrosis,” refers to the growth of scar tissue in the liver that can occur in the context of a chronic hepatitis infection.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a continuous delivery device (e.g., a pump or other controlled release injectible system); and a single subcutaneous injection followed by installation of a continuous delivery system.

“Continuous delivery” as used herein (e.g., in the context of “continuous delivery of a substance to a tissue”) is meant to refer to movement of drug to a delivery site, e.g., into a tissue in a fashion that provides for delivery of a desired amount of substance into the tissue over a selected period of time, where about the same quantity of drug is received by the patient each minute during the selected period of time.

“Controlled release” as used herein (e.g., in the context of “controlled drug release”) is meant to encompass release of substance (e.g., a Type I or Type III interferon receptor agonist, e.g., IFN-α) at a selected or otherwise controllable rate, interval, and/or amount, which is not substantially influenced by the environment of use. “Controlled release” thus encompasses, but is not necessarily limited to, substantially continuous delivery, and patterned delivery (e.g., intermittent delivery over a period of time that is interrupted by regular or irregular time intervals).

“Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unlit time, or volume of drug formulation for a unit time), and further encompasses delivery that is continuous or substantially continuous, or chronic.

The term “controlled drug delivery device” is meant to encompass any device wherein the release (e.g., rate, timing of release) of a drug or other desired substance contained therein is controlled by or determined by the device itself and not substantially influenced by the environment of use, or releasing at a rate that is reproducible within the environment of use.

By “substantially continuous” as used in, for example, the context of “substantially continuous infusion” or “substantially continuous delivery” is meant to refer to delivery of drug in a maimer that is substantially uninterrupted for a pre-selected period of drug delivery, where the quantity of drug received by the patient during any 8 hour interval in the pre-selected period never falls to zero. Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

By “substantially steady state” as used in the context of a biological parameter that may vary as a function of time, it is meant that the biological parameter exhibits a substantially constant value over a time course, such that the area under the curve defined by the value of the biological parameter as a function of time for any 8 hour period during the time course (AUC₈ₕᵣ) is no more than about 20% above or about 20% below, and preferably no more than about 15% above or about 15% below, and more preferably no more than about 10% above or about 10% below, the average area under the curve of the biological parameter over an 8 hour period during the time course (AUC_{8hr average}). The AUC₈ₕᵣ average is defined as the quotient (q) of the area under the curve of the biological parameter over the entirety of the time course (AUCₜₒₜₐₗ) divided by the number of 8 hour intervals in the time course (t_{total1/3days}), i.e., q=(AUCₜₒₜₐₗ)/(t_{total1/3days}). For example, in the context of a serum concentration of a drug, the serum concentration of the drug is maintained at a substantially steady state during a time course when the area under the curve of serum concentration of the drug over time for any 8 hour period during the time course (AUC₈ₕᵣ) is no more than about 20% above or about 20% below the average area under the curve of serum concentration of the drug over an 8 hour period in the time course (AUC_{8hr average}), i.e., the AUC₈ₕᵣ is no more than 20% above or 20% below the AUC₈ₕᵣ average for the serum concentration of the drug over the time course.

As used herein, any compound or agent described as “effective for the avoidance or amelioration of side effects induced by a Type I interferon receptor agonist,” or as “effective for reducing or eliminating the severity or occurrence of side effects induced by a Type I interferon receptor agonist,” or any compound or agent described by language with a meaning similar or equivalent to that of either of the foregoing quoted passages, is/are defined as a compound(s) or agent(s) that when co-administered to a patient in an effective amount along with a given dosing regimen of a subject Type I interferon receptor agonist combination therapy, abates or eliminates the severity or occurrence of side effects experienced by a patient in response to the given dosing regimen of the a Type I interferon receptor agonist combination therapy, as compared to the severity or occurrence of side effects that would have been experienced by the patient in response to the same dosing regimen of the a Type I interferon receptor agonist combination therapy without co-administration of the agent.

In many embodiments, the effective amounts of a Type I interferon receptor agonist and a second therapeutic agent are synergistic amounts. As used herein, a “synergistic combination” or a “synergistic amount” of a Type I interferon receptor agonist and a second therapeutic agent is a combination or amount that is more effective in the therapeutic or prophylactic treatment of a disease than the incremental improvement in treatment outcome that could be predicted or expected from a merely additive combination of (i) the therapeutic or prophylactic benefit of the Type I interferon receptor agonist when administered at that same dosage as a monotherapy and (ii) the therapeutic or prophylactic benefit of the second therapeutic agent when administered at the same dosage as a monotherapy.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a Type I interferon receptor agonist” includes a plurality of such agonists and reference to “the NS3 inhibitor” includes reference to one or more NS3 inhibitors and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of treating hepatitis C virus (HCV) infection; methods of reducing the incidence of complications associated with HCV and cirrhosis of the liver; and methods of reducing viral load, or reducing the time to viral clearance, or reducing morbidity or mortality in the clinical outcomes, in patients suffering from HCV infection. The methods generally involve administering to the individual i) a Type I interferon receptor agonist or a Type III interferon receptor agonist; ii) an immunomodulatory agent; and iii) an inhibitor of an HCV enzyme.

Without intending a limitation to any particular mechanism, the present invention arises in part from the discernment of an adverse effect that the inhibition of NS3 will have on cytotoxic T lymphocyte (CTL)-mediated clearance of virally infected tissue. The adverse effect on CTL activity is believed to originate from the pleiotropic effects of Type I interferon receptor activation, which include the induction of TNF (and other inflammatory cytokines). Interferon response factor 3 (IRF3) mediates the transduction of signal from activated Type I interferon receptor. NS3 mediates HCV resistance to Type I interferon therapy by blocking the activation and effector function of IRF3 (Foy, et al., Science 300: 1145-1148 (2003)). Thus, the inhibition of NS3 will improve IRF3 -mediated signal transduction, leading to stronger TNF induction. The increase in TNF will create a cytokine pattern that disfavors TH1 activation, thereby reducing CTL-mediated clearance of virally infected tissue. The regimens of the present invention include an immunomodulator to moderate or offset the NS3 inhibitor's negative impact on CTL activity in HCV infection.

In addition, the above-described mechanism applies to intervention with an inhibitor of HCV NS5B RNA-dependent RNA polymerase (RDRP). Since RDRP activity is needed for replication of viral RNA template encoding NS3, the inhibition of RDRP activity will downregulate NS3 expression and improve IRF3-mediated induction of TNF. Thus, the immunomodulator in the present regimens will moderate or offset the RDRP inhibitor's negative impact on CTL activity in HCV infection.

Treatment Methods

The present invention provides methods of treating an HCV infection; and methods of treating complications or sequelae of an HCV infection, e.g., liver fibrosis. The methods generally involve a combination therapeutic regimen, wherein a Type I or a Type III interferon receptor agonist, one or more immunomodulatory agents, and one or more HCV enzyme inhibitors are administered to an individual in need thereof.

Hepatitis Virus Infections

The present invention provides methods for treating a hepatitis C virus (HCV) infection. The methods generally involve a combination therapy comprising administering to an individual in need thereof combined effective amounts of i) a Type I interferon receptor agonist or a Type III interferon receptor agonist; ii) an immunomodulatory agent; and iii) an inhibitor of an HCV enzyme, where the combination therapy is effective to decrease viral load in the individual, and to achieve a sustained viral response. Optionally, the subject method further provides administering to the individual an effective amount of a nucleoside analog, such as ribavirin, levovirin, and viramidine. Of particular interest in many embodiments is treatment of humans. In many embodiments, the Type I interferon receptor agonist is IFN-α.

Whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

Viral load can be measured by measuring the titer or level of virus in serum. These methods include, but are not limited to, a quantitative polymerase chain reaction (PCR) and a branched DNA (bDNA) test. Quantitative assays for measuring the viral load (titer) of HCV RNA have been developed. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif. See, e.g., Gretch et al. (1995) Ann. Intern. Med. 123:321-329.

In general, an effective amount of a therapeutic agent that is administered as part of a subject combination therapy is an amount that is effective to reduce viral load to undetectable levels, e.g., to less than about 5000, less than about 1000, less than about 500, or less than about 200 genome copies/mL serum. In some embodiments, an effective amount of a therapeutic agent that is administered as part of a subject combination therapy is an amount that is effective to reduce viral load to less than 100 genome copies/mL serum. In many embodiments, the methods of the invention achieve a sustained viral response, e.g., the viral load is reduced to undetectable levels for a period of at least about one month, at least about two months, at least about three months, at least about four months, at least about five months, or at least about six months following cessation of treatment.

As noted above, whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of a therapeutic agent that is administered as part of a subject combination therapy is an amount effective to reduce ALT levels to less than about 45 U/ml serum.

A therapeutically effective amount of a therapeutic agent that is administered as part of a subject combination therapy is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., enzyme-linked immunosorbent assays (ELISA), radioimmunoassays, and the like, using antibody specific for a given serum marker.

Liver Fibrosis

Liver fibrosis is a precursor to the complications associated with liver cirrhosis, such as portal hypertension, progressive liver insufficiency, and hepatocellular carcinoma. A reduction in liver fibrosis thus reduces the incidence of such complications. Accordingly, the present invention further provides methods of reducing the likelihood that an individual will develop complications associated with cirrhosis of the liver.

The present methods generally involve administering therapeutically effective amounts of i) a Type I interferon receptor agonist or a Type III interferon receptor agonist; ii) an immunomodulatory agent; and iii) an inhibitor of an HCV enzyme. As used herein, “effective amounts” of therapeutic agents that are administered in a subject combination therapy are any combined dosage that is effective in reducing liver fibrosis or reduce the rate of progression of liver fibrosis; and/or that is effective in reducing the likelihood that an individual will develop liver fibrosis; and/or that is effective in reducing a parameter associated with liver fibrosis; and/or that is effective in reducing a disorder associated with cirrhosis of the liver.

The invention also provides a method for treatment of liver fibrosis in an individual comprising administering to the individual amounts of i) a Type I interferon receptor agonist or a Type III interferon receptor agonist; ii) an imununomodulatory agent; and iii) an inhibitor of an HCV enzyme that are effective for prophylaxis or therapy of liver fibrosis in the individual, e.g., increasing the probability of survival, reducing the risk of death, ameliorating the disease burden or slowing the progression of disease in the individual.

Whether treatment with a combination of i) a Type I interferon receptor agonist or a Type III interferon receptor agonist; ii) an immunomodulatory agent; and iii) an inhibitor of an HCV enzyme is effective in reducing liver fibrosis can be determined by any of a number of well-established techniques for measuring liver fibrosis and liver function. Whether liver fibrosis is reduced is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, therapeutically effective amounts of a Type I or III interferon receptor agonist, an immunomodulatory agent, and an HCV enzyme inhibitor are any combined dosage that effects a change of one unit or more in the fibrosis stage based on pre- and post-therapy liver biopsies. In particular embodiments, therapeutically effective amounts of a Type I or III interferon receptor agonist, an immunomodulatory agent, and an HCV enzyme inhibitor reduce liver fibrosis by at least one unit in the METAVIR, the Inodell, the Scheuer, the Ludwig, or the Ishalc scoring system.

Secondary, or indirect, indices of liver function can also be used to evaluate the efficacy of treatment with a subject combination therapy. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

In another embodiment, effective amounts of i) a Type I interferon receptor agonist or a Type III interferon receptor agonist; ii) an immunomodulatory agent; and iii) an inhibitor of an HCV enzyme are any combined dosage that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or in a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

In another embodiment, therapeutically effective amounts of i) a Type I interferon receptor agonist or a Type III interferon receptor agonist; ii) an immunomodulatory agent; and iii) an inhibitor of an HCV enzyme are any combined dosage that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or in a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linled immunosorbent assays (ELISA), radioimmunoassays, and the like, using antibody specific for a given serum marker.

Quantitative tests of functional liver reserve can also be used to assess the efficacy of treatment with dual IFN receptor agonist therapy. These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequelae of decompensated liver disease, i.e., or occurs subsequently to and as a result of development of liver fibrosis, and includes, but is not limited to, development of ascites, variceal bleeding, portal hypertension, jaundice, progressive liver insufficiency, encephalopathy, hepatocellular carcinoma, liver failure requiring liver transplantation, and liver-related mortality.

In another embodiment, therapeutically effective amounts of i) a Type I interferon receptor agonist or a Type III interferon receptor agonist; ii) an immunomodulatory agent; and iii) an inhibitor of an HCV enzyme are any combined dosage that is effective in reducing the incidence of (e.g., the likelihood that an individual will develop) a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or in a placebo-treated individual.

Whether combination therapy with a Type I or III interferon receptor agonist, an immunomodulatory agent and an HCV enzyme inhibitor is effective in reducing the incidence of a disorder associated with cirrhosis of the liver can readily be determined by those skilled in the art.

Reduction in liver fibrosis increases liver function. Thus, the invention provides methods for increasing liver function, generally involving administering therapeutically effective amounts of i) a Type I interferon receptor agonist or a Type III interferon receptor agonist; ii) an immunomodulatory agent; and iii) an inhibitor of an HCV enzyme. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal range of alanine transaminase is from about 7 to about 56 units per liter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

In another embodiment, therapeutically effective amounts of i) a Type I interferon receptor agonist or a Type III interferon receptor agonist; ii) an immunomodulatory agent; and iii) an inhibitor of an HCV enzyme are any combined dosage that is effective to increase liver function by at least about 10%, at least about 20%, at least about 3 0%, at least about 40%, at least about 50%, at least about 60%, at least about 70%, at least about 80%, or more. In one non-limiting example, therapeutically effective amounts of i) a Type I interferon receptor agonist or a Type III interferon receptor agonist; ii) an immunomodulatory agent; and iii) an inhibitor of an HCV enzyme are any combined dosage that is effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to withhiin a normal range. In another non-limiting example, therapeutically effective amounts of i) a Type I interferon receptor agonist or a Type III interferon receptor agonist; ii) an immunomodulatory agent; and iii) an inhibitor of an HCV enzyme any combined dosage effective to increase a reduced level of a serum marker of liver ftmction by at least about 10%, at least about 20%, at least about 30%, at least about 40%, at least about 50%, at least about 60%, at least about 70%, at least about 80%, or more, or to increase the level of the serum marker of liver function to within a normal range.

Type I Interferon Receptor Agonists

In any of the above-described methods, in some embodiments a Type I interferon receptor agonist is administered. Type I interferon receptor agonists include an IFN-α; an IFN-β an IFN-tau; an IFN-ω; antibody agonists specific for a Type I interferon receptor; and any other agonist of Type I interferon receptor, including non-polypeptide agonists.

Interferon-Alpha

Any known IFN-α can be used in the instant invention. The term “interferon-alpha” as used herein refers to a family of related polypeptides that inhibit viral replication and cellular proliferation and modulate immune response. The term “IFN-α” includes naturally occurring IFN-α; synthetic IFN-α; derivatized IFN-α (e.g., PEGylated IFN-α, glycosylated IFN-α, and the like); and analogs of naturally occurring or synthetic IFN-α; essentially any IFN-α that has antiviral properties, as described for naturally occurring IFN-α.

Suitable alpha interferons include, but are not limited to, naturally-occurring IFN-α (including, but not limited to, naturally occurring IFN-α2a, IFN-α2b); recombinant interferon alpha-2b such as Intron-A interferon available from Schering Corporation, Kenilworth, N.J.; recombinant interferon alpha-2a such as Roferon interferon available from Hoffmann-La Roche, Nutley, N.J.; recombinant interferon alpha-2C such as Berofor alpha 2 interferon available from Boehringer Ingelheim Pharmaceutical, Inc., Ridgefield, Conn.; interferon alpha-n1, a purified blend of natural alpha interferons such as Sumiferon available from Sumitomo, Japan or as Weliferon interferon alpha-n1 (INS) available from the Glaxo-Wellcome Ltd., London, Great Britain; and interferon alpha-n3 a mixture of natural alpha interferons made by Interferon Sciences and available from the Purdue Frederick Co., Norwalk, Conn., under the Alferon Tradename.

The term “IFN-α” also encompasses consensus IFN-α. Consensus IFN-α (also referred to as “CIFN” and “IFN-con” and “consensus interferon”) encompasses but is not limited to the amino acid sequences designated IFN-con₁, IFN-con₂ and IFN-con₃ which are disclosed in U.S. Pat. Nos. 4,695,623 and 4,897,471; and consensus interferon as defined by determination of a consensus sequence of naturally occurring interferon alphas (e.g., INFERGEN®), InterMune, Inc., Brisbane, Calif.). IFN-con₁ is the consensus interferon agent in the INFERGEN® alfacon-1 product. The INFERGEN® consensus interferon product is referred to herein by its brand name (INFERGEN®) or by its generic name (interferon alfacon-1). DNA sequences encoding IFN-con may be synthesized as described in the aforementioned patents or other standard methods. Use of CIFN is of particular interest.

Also suitable for use in the present invention are fusion polypeptides comprising an IFN-α and a heterologous polypeptide. Suitable IFN-α fusion polypeptides include, but are not limited to, Albuferon-alpha™ (a fusion product of human albumin and IFN-α; Human Genome Sciences; see, e.g., Osborn et al. (2002) J. Pharmacol. Exp. Therap. 303:540-548). Also suitable for use in the present invention are gene-shuffled forms of IFN-α. See., e.g., Masci et al. (2003) Curr. Oncol. Rep. 5:108-113.

PEGylated Interferon-Alpha

The term “IFN-α” also encompasses derivatives of IFN-α that are derivatized (e.g., are chemically modified) to alter certain properties such as serum half-life. As such, the term “IFN-α” includes glycosylated IFN-α; IFN-α derivatized with polyethylene glycol (“PEGylated IFN-α”); and the like. PEGylated IFN-α, and methods for making same, is discussed in, e.g., U.S. Pat. Nos. 5,382,657; 5,981,709; and 5,951,974. PEGylated IFN-α encompasses conjugates of PEG and any of the above-described IFN-α molecules, including, but not limited to, PEG conjugated to interferon alpha-2a (Roferon, Hoffman La-Roche, Nutley, N.J.), interferon alpha 2b (Intron, Schering-Plough, Madison, N.J.), interferon alpha-2c (Berofor Alpha, Boebringer Ingelheim, Ingelheim, Germany); and consensus interferon as defined by determination of a consensus sequence of naturally occurring interferon alphas (INFERGEN(®, InterMune, Inc., Brisbane, Calif.).

Any of the above-mentioned IFN-α polypeptides can be modified with one or more polyethylene glycol moieties, i.e., PEGylated. The PEG molecule of a PEGylated IFN-α polypeptide is conjugated to one or more amino acid side chains of the IFN-α polypeptide. In some embodiments, the PEGylated IFN-α contains a PEG moiety on only one amino acid. In other embodiments, the PEGylated IFN-α contains a PEG moiety on two or more amino acids, e.g., the IFN-α contains a PEG moiety attached to two, three, four, five, six, seven, eight, nine, or ten different amino acid residues.

IFN-α may be coupled directly to PEG (i.e., without a linking group) through an amino group, a sulfhydryl group, a hydroxyl group, or a carboxyl group.

In some embodiments, the PEGylated IFN-α is PEGylated at or near the amino terminus (N-terminus) of the IFN-α polypeptide, e.g., the PEG moiety is conjugated to the IFN-α polypeptide at one or more amino acid residues from amino acid 1 through amino acid 4, or from amino acid 5 through about 10.

In other embodiments, the PEGylated IFN-α is PEGylated at one or more amino acid residues from about 10 to about 28.

In other embodiments, the PEGylated IFN-α is PEGylated at or near the carboxyl terminus (C-terminus) of the IFN-α polypeptide, e.g., at one or more residues from amino acids 156-166, or from amino acids 150 to 155.

In other embodiments, the PEGylated IFN-α is PEGylated at one or more amino acid residues at one or more residues from amino acids 100-114.

The polyethylene glycol derivatization of amino acid residues at or near the receptor-binding and/or active site domains of the IFN-α protein can disrupt the functioning of these domains. In certain embodiments of the invention, amino acids at which PEGylation is to be avoided include amino acid residues from amino acid 30 to amino acid 40; and amino acid residues from amino acid 113 to amino acid 149.

In some embodiments, PEG is attached to IFN-α via a linking group. The linking group is any biocompatible linking group, where “biocompatible” indicates that the compound or group is non-toxic and may be utilized in vitro or in vivo without causing injury, sickness, disease, or death. PEG can be bonded to the linking group, for example, via an ether bond, an ester bond, a thiol bond or an amide bond. Suitable biocompatible linking groups include, but are not limited to, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl butanoate (SBA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteihe group, a histidine group or a primary amine.

Methods for making succinimidyl propionate (SPA) and succinimidyl butanoate (SBA) ester-activated PEGs are described in U.S. Pat. No. 5,672,662 (Harris, et al.) and WO 97/03106.

Methods for attaching a PEG to an IFN-α polypeptide are known in the art, and any known method can be used. See, for example, by Park et al, Anticancer Res., 1:373-376 (1981); Zaplipsky and Lee, Polyethylene Glycol Chemistry: Biotechnical and Biomedical Applications, J. M. Harris, ed., Plenum Press, NY, Chapter 21 (1992); U.S. Pat. No. 5,985,265; U.S. Pat. No. 5,672,662 (Harris, et al.) and WO 97/03106.

Pegylated IFN-α, and methods for making same, is discussed in, e.g., U.S. Pat. Nos. 5,382,657; 5,981,709; 5,985,265; and 5,951,974. Pegylated IFN-α encompasses conjugates of PEG and any of the above-described IFN-α molecules, including, but not limited to, PEG conjugated to interferon alpha-2a (Roferon, Hoffman LaRoche, Nutley, N.J.), where PEGylated Roferon is known as Pegasys (Hoffman LaRoche); interferon alpha 2b (Intron, Schering-Plough, Madison, N.J.), where PEGylated Intron is known as PEG-Intron (Schering-Plough); interferon alpha-2c (Berofor Alpha, Boehringer Ingelheim, Ingelheim, Germany); and consensus interferon (CIFN) as defined by determination of a consensus sequence of naturally occurring interferon alphas (INFERGEN®, InterMune, Inc., Brisbane, Calif.), where PEGylated CIFN is referred to as PEG-CIFN.

In many embodiments, the PEG is a monomethoxyPEG molecule that reacts with primary amine groups on the IFN-α polypeptide. Methods of modifying polypeptides with monomethoxy PEG via reductive alkylation are known in the art. See, e.g., Chamow et al. (1994) Bioconj. Chem. 5:133-140.

In one non-limiting example, PEG is linked to IFN-α via an SPA linking group. SPA esters of PEG, and methods for making same, are described in U.S. Pat. No. 5,672,662. SPA linkages provide for linkage to free amine groups on the IFN-α polypeptide.

For example, a PEG molecule is covalently attached via a linkage that comprises an amide bond between a propionyl group of the PEG moiety and the epsilon amino group of a surface-exposed lysine residue in the IFN-α polypeptide. Such a bond can be formed, e.g., by condensation of an α-methoxy, omega propanoic acid activated ester of PEG (mPEGspa).

In some embodiments, the invention employs a PEG-modified CIFN, where the PEG moiety is attached to a lysine residue chosen from lys³¹, lys⁵⁰, lys⁷¹, lys⁸⁴, lys¹²¹, lys122, lys¹³⁴, lys¹³⁵, and lys¹⁶⁵. In these embodiments, the PEG moiety can be a linear PEG moiety having an average molecular weight of about 30 kD.

In other embodiments, the invention employs a PEG-modified CIFN, where the PEG moiety is attached to a lysine residue chosen from lys¹²¹, lys¹³⁴, lys¹³⁵, and lys¹⁶⁵. In these embodiments, the PEG moiety can be a linear PEG moiety having an average molecular weight of about 30 kD.

As one non-limiting example, one monopegylated CIFN conjugate preferred for use herein has a linear PEG moiety of about 30 kD attached via a covalent linkage to the CIFN polypeptide, where the covalent linkage is an amide bond between a propionyl group of the PEG moiety and the epsilon amino group of a surface-exposed lysine residue in the CIFN polypeptide, where the surface-exposed lysine residue is chosen from lys³¹, lys⁵⁰, lys⁷¹, lys⁸⁴, lys¹²¹, lys¹²², lys¹³⁴, lys¹³⁵, and lys¹⁶⁵, and the amide bond is formed by condensation α-methoxy, omega propanoic acid activated ester of PEG.

Linking Groups

In some embodiments, PEG is attached to IFN-α via a linking group. The linking group is any biocompatible linking group, where “biocompatible” indicates that the compound or group is essentially non-toxic and may be utilized in vivo without causing a significant adverse response in the subject, e.g., injury, sickness, disease, undesirable immune response, or death. PEG can be bonded to the linking group, for example, via an ether bond, an ester bond, a thio ether bond or an amide bond. Suitable biocompatible linking groups include, but are not limited to, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl butanoic acid (SBA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine.

In many embodiments, the PEG is a monomethoxyPEG molecule that reacts with primary amine groups on the IFN-α polypeptide. Methods of modifying polypeptides with monomethoxy PEG via reductive allcylation are known in the art. See, e.g., Chamow et al. (1994) Bioconj. Chem. 5:133-140.

In one non-limiting example, PEG is linked to IFN-α via an SPA linking group. SPA esters of PEG, and methods for making same, are described in U.S. Pat. No. 5,672,662. SPA linkages provide for linkage to free amine groups on the IFN-α polypeptide.

For example, a PEG molecule is covalently attached via a linkage that comprises an amide bond between a propionyl group of the PEG moiety and the epsilon amino group of a surface-exposed lysine residue in the IFN-α polypeptide. Such a bond can be formed, e.g., by condensation of an a-methoxy, omega propanoic acid activated ester of PEG (mPEGspa).

As one non-limiting example, monopegylated CIFN has a linear PEG moiety of about 30 kD attached via a covalent linkage to the CIFN polypeptide, where the covalent linkage is an amide bond between a propionyl group of the PEG moiety and the epsilon amino group of a surface-exposed lysine residue in the CIFN polypeptide, where the surface-exposed lysine residue is chosen from lys¹²¹, lys¹³⁴, lys¹³⁵, and lys¹⁶⁵, and the amide bond is formed by condensation of an a-methoxy, omega propanoic acid activated ester of PEG.

Methods for attaching a PEG molecule to an IFN-α polypeptide are known in the art, and any known method can be used. See, for example, by Park et al, Anticancer Res., 1:373-376 (1981); Zaplipsky and Lee, Polyethylene Glycol Chemistry: Biotechnical and Biomedical Applications, J. M. Harris, ed., Plenwn Press, NY, Chapter 21 (1992); and U.S. Pat. No. 5,985,265.

Polyethylene Glycol

Polyethylene glycol suitable for conjugation to an IFN-α polypeptide is soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons.

In many embodiments, PEG has at least one hydroxyl group, e.g., a terminal hydroxyl group, which hydroxyl group is modified-to generate a functional group that is reactive with an amino group, e.g., an epsilon amino group of a lysine residue, a free amino group at the N-terminus of a polypeptide, or any other amino group such as an amino group of asparagine, glutamine, arginine, or histidine.

In other embodiments, PEG is derivatized so that it is reactive with free carboxyl groups in the IFN-α polypeptide, e.g., the free carboxyl group at the carboxyl terminus of the IFN-α polypeptide. Suitable derivatives of PEG that are reactive with the free carboxyl group at the carboxyl-terminus of IFN-α include, but are not limited to PEG-amine, and hydrazine derivatives of PEG (e.g., PEG-NH—NH₂).

In other embodiments, PEG is derivatized such that it comprises a terminal thiocarboxylic acid group, —COSH, which selectively reacts with amino groups to generate amide derivatives. Because of the reactive nature of the thio acid, selectivity of certain amino groups over others is achieved. For example, —SH exhibits sufficient leaving group ability in reaction with N-terminal amino group at appropriate pH conditions such that the ε-amino groups in lysine residues are protonated and remain non-nucleophilic. On the other hand, reactions under suitable pH conditions may make some of the accessible lysine residues to react with selectivity.

In other embodiments, the PEG comprises a reactive ester such as an N-hydroxy succinimidate at the end of the PEG chain. Such an N-hydroxysuccinimidate-containing PEG molecule reacts with select amino groups at particular pH conditions such as neutral 6.5-7.5. For example, the N-terminal amino groups may be selectively modified under neutral pH conditions. However, if the reactivity of the reagent were extreme, accessible-NH₂ groups of lysine may also react.

The PEG can be conjugated directly to the IFN-α polypeptide, or through a linker. In some embodiments, a linker is added to the IFN-α polypeptide, forming a linker-modified IFN-α polypeptide. Such linkers provide various functionalities, e.g., reactive groups such sulfhydryl, amino, or carboxyl groups to couple a PEG reagent to the linker-modified IFN-α polypeptide.

In some embodiments, the PEG conjugated to the IFN-α polypeptide is linear. In other embodiments, the PEG conjugated to the IFN-α polypeptide is branched. Branched PEG derivatives such as those described in U.S. Pat. No. 5,643,575, “star-PEG's” and multi-armed PEG's such as those described in Shearwater Polymers, Inc. catalog “Polyethylene Glycol Derivatives 1997-1998.” Star PEGs are described in the art including, e.g., in U.S. Pat. No. 6,046,305.

PEG having a molecular weight in a range of from about 2 kDa to about 100 kDa, is generally used, where the term “about,” in the context of PEG, indicates that in preparations of polyethylene glycol, some molecules will weigh more, some less, than the stated molecular weight. For example, PEG suitable for conjugation to IFN-α has a molecular weight of from about 2 kDa to about 5 kDa, from about 5 kDa to about 10 kDa, from about 10 kDa to about 15 kDa, from about 15 kDa to about 20 kDa, from about 20 kDa to about 25 kDa, from about 25 kDa to about 30 kDa, from about 30 kDa to about 40 kDa, from about 40 kDa to about 50 kDa, from about 50 kDa to about 60 kDa, from about 60 kDa to about 70 kDa, from about 70 kDa to about 80 kDa, from about 80 kDa to about 90 kDa, or from about 90 kDa to about 100 kDa.

Preparing PEG-IFN-α Conjugates

As discussed above, the PEG moiety can be attached, directly or via a linker, to an amino acid residue at or near the N-terminus, internally, or at or near the C-terminus of the IFN-α polypeptide. Conjugation can be carried out in solution or in the solid phase.

N-Terminal Linkage

Methods for attaching a PEG moiety to an amino acid residue at or near the N-terminus of an IFN-α polypeptide are known in the art. See, e.g., U.S. Pat. No. 5,985,265.

In some embodiments, known methods for selectively obtaining an N-terminally chemically modified IFN-α are used. For example, a method of protein modification by reductive alkylation which exploits differential reactivity of different types of primary amino groups (lysine versus the N-terminus) available for derivatization in a particular protein can be used. Under the appropriate reaction conditions, substantially selective derivatization of the protein at the N-terminus with a carbonyl group containing polymer is achieved. The reaction is performed at pH which allows one to take advantage of the pKₐ differences between the ε-amino groups of the lysine residues and that of the α-amino group of the N-terminal residue of the protein. By such selective derivatization attachment of a PEG moiety to the IFN-α is controlled: the conjugation with the polymer takes place predominantly at the N-terminus of the IFN-α and no significant modification of other reactive groups, such as the lysine side chain amino groups, occurs.

C-terminal Linkage

N-terminal-specific coupling procedures such as described in U.S. Pat. No. 5,985,265 provide predominantly monoPEGylated products. However, the purification procedures aimed at removing the excess reagents and minor multiply PEGylated products remove the N-terminal blocked polypeptides. In terms of therapy, such processes lead to significant increases in manufacturing costs. For example, examination of the structure of the well-characterized INFERGEN® Alfacon-1 CIFN polypeptide amino acid sequence reveals that the clipping is approximate 5% at the carboxyl terminus and thus there is only one major C-terminal sequence. Thus, in some embodiments, N-terminally PEGylated IFN-α is not used; instead, the IFN-α polypeptide is C-terminally PEGylated.

An effective synthetic as well as therapeutic approach to obtain mono PEGylated INFERGEN product is therefore envisioned as follows:

A PEG reagent that is selective for the C-terminal can be prepared with or without spacers. For example, polyethylene glycol modified as methyl ether at one end and having an amino function at the other end may be used as the starting material.

Preparing or obtaining a water-soluble carbodiimide as the condensing agent can be carried out. Coupling IFN-α (e.g., INFERGEN® Alfacon-1 CIFN or consensus interferon) with a water-soluble carbodiimide as the condensing reagent is generally carried out in aqueous medium with a suitable buffer system at an optimal pH to effect the amide linkage. A high molecular weight PEG can be added to the protein covalently to increase the molecular weight.

The reagents selected will depend on process optimization studies. A non-limiting example of a suitable reagent is EDAC or 1-ethyl-3-(3-dimethylaminopropyl) carbodiimide. The water solubility of EDAC allows for direct addition to a reaction without the need for prior organic solvent dissolution. Excess reagent and the isourea formed as the by-product of the cross-linking reaction are both water-soluble and may easily be removed by dialysis or gel filtration. A concentrated solution of EDAC in water is prepared to facilitate the addition of a small molar amount to the reaction. The stock solution is prepared and used immediately in view of the water labile nature of the reagent. Most of the synthetic protocols in literature suggest the optimal reaction medium to be in pH range between 4.7 and 6.0. However the condensation reactions do proceed without significant losses in yields up to pH 7.5. Water may be used as solvent. In view of the contemplated use of INFERGEN, preferably the medium will be 2-(N-morpholino)ethane sulfonic acid buffer pre-titrated to pH between 4.7 and 6.0. However, 0.1M phosphate in the pH 7-7.5 may also be used in view of the fact that the product is in the same buffer. The ratios of PEG amine to the IFN-α molecule is optimized such that the C-terminal carboxyl residue(s) are selectively PEGylated to yield monoPEGylated derivative(s).

Even though the use of PEG amine has been mentioned above by name or structure, such derivatives are meant to be exemplary only, and other groups such as hydrazine derivatives as in PEG-NH—NH₂ which will also condense with the carboxyl group of the IFN-α protein, can also be used. In addition to aqueous phase, the reactions can also be conducted on solid phase. Polyethylene glycol can be selected from list of compounds of molecular weight ranging from 300-40000. The choice of the various polyethylene glycols will also be dictated by the coupling efficiency and the biological performance of the purified derivative in vitro and in vivo i.e., circulation times, anti viral activities etc.

Additionally, suitable spacers can be added to the C-terminal of the protein. The spacers may have reactive groups such as SH, NH₂ or COOH to couple with appropriate PEG reagent to provide the high molecular weight IFN-α derivatives. A combined solid/solution phase methodology can be devised for the preparation of C-terminal pegylated interferons. For example, the C-terminus of IFN-α is extended on a solid phase using a Gly-Gly-Cys-NH₂ spacer and then monopegylated in solution using activated dithiopyridyl-PEG reagent of appropriate molecular weights. Since the coupling at the C-terminus is independent of the blocking at the N-terminus, the envisioned processes and products will be beneficial with respect to cost (a third of the protein is not wasted as in N-terminal PEGylation methods) and contribute to the economy of the therapy to treat chronic hepatitis C infections, liver fibrosis etc.

There may be a more reactive carboxyl group of amino acid residues elsewhere in the molecule to react with the PEG reagent and lead to monoPEGylation at that site or lead to multiple PEGylations in addition to the —COOH group at the C-terminus of the IFN-α. It is envisioned that these reactions will be minimal at best owing to the steric freedom at the C-terminal end of the molecule and the steric hindrance imposed by the carbodiimides and the PEG reagents such as in branched chain molecules. It is therefore the preferred mode of PEG modification for INFERGEN and similar such proteins, native or expressed in a host system, which may have blocked N-termini to varying degrees to improve efficiencies and maintain higher in vivo biological activity.

Another method of achieving C-terminal PEGylation is as follows. Selectivity of C-terminal PEGylation is achieved with a sterically hindered reagent which excludes reactions at carboxyl residues either buried in the helices or internally in IFN-α. For example, one such reagent could be a branched chain PEG 40kd in molecular weight and this agent could be synthesized as follows: OH₃C—(CH₂CH₂O)n-CH₂CH₂NH₂+ Glutamic Acid i.e., HOCO—CH₂CH₂CH(NH₂)—COOH is condensed with a suitable agent e.g., dicyclohexyl carbodiimide or water-soluble EDC to provide the branched chain PEG agent OH₃C—(CH₂CH₂O)ₙ-CH₂CH₂NHCOCH(NH₂)CH₂OCH₃—(CH₂CH₂O)ₙ-CH₂CH₂NHCOCH₂.

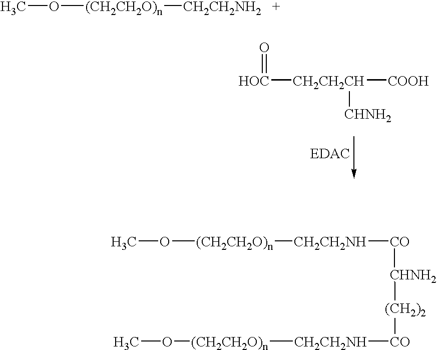

This reagent can be used in excess to couple the amino group with the free and flexible carboxyl group of IFN-α to form the peptide bond.

If desired, PEGylated IFN-α is separated from unPEGylated IFN-α using any known method, including, but not limited to, ion exchange chromatography, size exclusion chromatography, and combinations thereof. For example, where the PEG-IFN-α conjugate is a monoPEGylated IFN-α, the products are first separated by ion exchange chromatography to obtain material having a charge characteristic of monoPEGylated material (other multi-PEGylated material having the same apparent charge may be present), and then the monoPEGylated materials are separated using size exclusion chromatography.

MonoPEG (30 kD, linear)-ylated IFN-α

PEGylated IFN-α that is suitable for use in the present invention includes a monopegylated consensus interferon (CIFN) molecule comprised of a single CIFN polypeptide and a single polyethylene glycol (PEG) moiety, where the PEG moiety is linear and about 30 kD in molecular weight and is directly or indirectly linked through a stable covalent link-age to either the N-terminal residue in the CIFN polypeptide or a lysine residue in the CIFN polypeptide. In some embodiments, the monoPEG (30 kD, linear)-ylated IFN-α is monoPEG (30 kD, linear)-ylated consensus IFN-α.

In some embodiments, the PEG moiety is linked to either the alpha-amino group of the N-terminal residue in the CIFN polypeptide or the epsilon-amino group of a lysine residue in the CIFN polypeptide. In further embodiments, the linkage comprises an amide bond between the PEG moiety and either the alpha-amino group of the N-terminal residue or the epsilon-amino group of the lysine residue in the CIFN polypeptide. In still further embodiments, the linkage comprises an amide bond between a propionyl group of the PEG moiety and either the alpha-amino group of the N-terminal residue or the epsilon-amino group of the lysine residue in the CIFN polypeptide. In additional embodiments, the amide bond is formed by condensation of an alpha-methoxy, omega-propanoic acid activated ester of the PEG moiety and either the alpha-amino group of the N-terminal residue or the epsilon-amino group of the lysine residue in the CIFN polypeptide, thereby forming a hydrolytically stable linkage between the PEG moiety and the CIFN polypeptide.

In some embodiments, the PEG moiety is linked to the N-terminal residue in the CIFN polypeptide. In other embodiments, the PEG moiety is linked to the alpha-amino group of the N-terminal residue in the CIFN polypeptide. In further embodiments, the linkage comprises an amide bond between the PEG moiety and the alpha-amino group of the N-terminal residue in the CIFN polypeptide. In still further embodiments, the linkage comprises an amide bond between a propionyl group of the PEG moiety and the alpha-amino group of the N-terminal residue in the CIFN polypeptide. In additional embodiments, the amide bond is formed by condensation of an alpha-methoxy, omega-propanoic acid activated ester of the PEG moiety and the alpha-amino group of the N-terminal residue of the CIFN polypeptide.

In some embodiments, the PEG moiety is linked to a lysine residue in the CIFN polypeptide. In other embodiments, the PEG moiety is linked to the epsilon-amino group of a lysine residue in the CIFN polypeptide. In further embodiments, the linkage comprises an amide bond between the PEG moiety and the epsilon-amino group of the lysine group in the CIFN polypeptide. In still further embodiments, the linkage comprises an amide bond between a propionyl group of the PEG moiety and the epsilon-amino group of the lysine group in the CIFN polypeptide. In additional embodiments, the amide bond is formed by condensation of an alpha-methoxy, omega-propanoic acid activated ester of the PEG moiety and the epsilon-amino group of the lysine residue in the CIFN polypeptide.

In some embodiments, the PEG moiety is linked to a surface-exposed lysine residue in the CIFN polypeptide. In other embodiments, the PEG moiety is linked to the epsilon-amino group of a surface-exposed lysine residue in the CIFN polypeptide. In further embodiments, the linkage comprises an amide bond between the PEG moiety and the epsilon-amino group of the surface-exposed lysine residue in the CIFN polypeptide. In still further embodiments, the linkage comprises an amide bond between a propionyl group of the PEG moiety and the epsilon-amino group of the surface-exposed lysine residue in the CIFN polypeptide. In additional embodiments, the amide bond is formed by condensation of an alpha-methoxy, onlega-propanoic acid activated ester of the PEG moiety and the epsilon-amino group of the surface-exposed lysine residue in the CIFN polypeptide.

In some embodiments, the PEG moiety is linked to a lysine chosen from lys³¹, lys⁵⁰, lys⁷¹, lys⁸⁴, lys¹²¹, lys¹²², lys¹³⁴, lys¹³⁵, and lys¹⁶⁵ of the CIFN polypeptide. In other embodiments, the PEG moiety is linked to the epsilon-amino group of a lysine chosen from lys³¹, lys⁵⁰, lys⁷¹, lys⁸⁴, lys¹²¹, lys¹²², lys¹³⁴, lys¹³⁵, and lys¹⁶⁵ of the CIFN polypeptide. In further embodiments, the linkage comprises an amide bond between the PEG moiety and the epsilon-amino group of the chosen lysine residue in the CIFN polypeptide. In still further embodiments, the linkage comprises an amide bond between a propionyl group of the PEG moiety and the epsilon-amino group of the chosen lysine residue in the CIFN polypeptide. In additional embodiments, the amide bond is formed by condensation of an alpha-methoxy, omega-propanoic acid activated ester of the PEG moiety and the epsilon-amino group of the chosen lysine residue in the CIFN polypeptide.

In some embodiments, the PEG moiety is linked to a lysine chosen from lys121, lys¹³⁴, lys¹³⁵, and lys¹⁶⁵ of the CIFN polypeptide. In other embodiments, the PEG moiety is linked to the epsilon-amino group of a lysine chosen from lys¹²¹, lys¹³⁴, lys¹³⁵, and lys¹⁶⁵ of the CIFN polypeptide. In further embodiments, the linkage comprises an amide bond between the PEG moiety and the epsilon-amino group of the chosen lysine residue in the CIFN polypeptide. In still further embodiments, the linkage comprises an amide bond between a propionyl group of the PEG moiety and the epsilon-amino group of the chosen lysine residue in the CIFN polypeptide. In additional embodiments, the amide bond is formed by condensation of an alpha-methoxy, omega-propanoic acid activated ester of the PEG moiety and the epsilon-amino group of the chosen lysine residue in the CIFN polypeptide.

In connection with the above-described monopegylated CIFN molecules, the invention contemplates embodiments of each such molecule where the CIFN polypeptide is chosen from interferon alpha-con₁, interferon alpha-con₂, and interferon alpha-con₃, the amino acid sequences of which CIFN polypeptides are disclosed in U.S. Pat. No. 4,695,623.

Populations of IFN-α

In addition, any of the methods of the invention can employ a PEGylated IFN-α composition that comprises a population of monopegylated IFN-α molecules, where the population consists of one or more species of monopegylated IFN-α molecules as described above. The subject composition comprises a population of modified IFN-α polypeptides, each with a single PEG molecule linked to a single amino acid residue of the polypeptide.

In some of these embodiments, the population comprises a mixture of a first IFN-α polypeptide linked to a PEG molecule at a first amino acid residue; and at least a second IFN-α polypeptide linked to a PEG molecule at a second amino acid residue, wherein the first and second IFN-α polypeptides are the same or different, and wherein the location of the first amino acid residue in the amino acid sequence of the first IFN-α polypeptide is not the same as the location of the second amino acid residue in the second IFN-α polypeptide. As one non-limiting example, an IFN-α composition suitable for use in a subject method comprises a population of PEG-modified IFN-α polypeptides, the population comprising an IFN-α polypeptide linked at its amino terminus to a linear PEG molecule; and an IFN-α polypeptide linked to a linear PEG molecule at a lysine residue.

Generally, a given modified IFN-α species represents from about 0.5% to about 99.5% of the total population of monopegylated IFNA polypeptide molecules in a population, e.g, a given modified IFN-α species represents about 0.5%, about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 99.5% of the total population of monopegylated IFN-α polypeptide molecules in a population. In some embodiments, a suitable composition comprises a population of monopegylated IFN-α polypeptides, which population comprises at least about 70%, at least about 80%, at least about 90%, at least about 95%, or at least about 99%, IFN-α polypeptides linled to PEG at the same site, e.g., at the N-terminal amino acid.

In particular embodiments of interest, a composition suitable for use in a subject method comprises a population of monopegylated CIFN molecules, the population consisting of one or more species of molecules, where each species of molecules is characterized by a single CIFN polypeptide linked, directly or indirectly in a covalent linkage, to a single linear PEG moiety of about 30 kD in molecular weight, and where the linkage is to either a lysine residue in the CIFN polypeptide, or the N-terminal amino acid residue of the CIFN polypeptide.

The amino acid residue to which the PEG is attached is in many embodiments the N-terminal amino acid residue. In other embodiments, the PEG moiety is attached (directly or via a linker) to a surface-exposed lysine residue. In additional embodiments, the PEG moiety is attached (directly or via a linker) to a lysine residue chosen from lys³¹, lys⁵⁰, lys⁷¹, lys⁸⁴, lys¹²¹, lys¹²², lys¹³⁴, lys¹³⁵, and lys¹⁶⁵ of the CIFN polypeptide. In further embodiments, the PEG moiety is attached (directly or via a linker) to a lysine residue chosen from lys¹²¹, lys¹³⁴, lys¹³⁵, and lys¹⁶⁵ of the CIFN polypeptide.

As an example, a composition suitable for use in a subject method comprises a population of monopegylated CIFN molecules, consisting of a first monopegylated CIFN polypeptide species of molecules characterized by a PEG moiety jinked at the N-terminal amino acid residue of a first CIFN polypeptide, and a second nionopegylated CIFN polypeptide species of molecules characterized by a PEG moiety linked to a first lysine residue of a second CIFN polypeptide, where the first and second CIFN polypeptides are the same or different. A composition suitable for use in a subject method can further comprise at least one additional monopegylated CIFN polypeptide species of molecules characterized by a PEG moiety linked to a lysine residue in the CIFN polypeptide, where the location of the linkage site in each additional monopegylated CIFN polypeptide species is not the same as the location of the linkage site in any other species. In all species in this example, the PEG moiety is a linear PEG moiety having an average molecular weight of about 30 kD.

As another example, a composition suitable for use in a subject method comprises a population of monopegylated CIFN molecules, consisting of a first monopegylated CIFN polypeptide species of molecules characterized by a PEG moiety linked at the N-terminal amino acid residue of a first CIFN polypeptide, and a second monopegylated CIFN polypeptide species of molecules characterized by a PEG moiety linked to a first surface-exposed lysine residue of a second CIFN polypeptide, where the first and second CIFN polypeptides are the same or different. A composition suitable for use in a subject method can further comprise at least one additional monopegylated CIFN polypeptide species of molecules characterized by a PEG moiety linked to a surface-exposed lysine residue in the CIFN polypeptide, where the location of the linkage site in each additional monopegylated CIFN polypeptide species is not the same as the location of the linkage site in any other species. In all species in this example, the PEG moiety is a linear PEG moiety having an average molecular weight of about 30 kD.

As another example, a composition suitable for use in a subject method comprises a population of monopegylated CIFN molecules, consisting of a first monopegylated CIFN polypeptide species of molecules characterized by a PEG moiety linked at the N-terminal amino acid residue of a first CIFN polypeptide, and a second monopegylated CIFN polypeptide species of molecules characterized by a PEG moiety linked to a first lysine residue selected from one of lys³¹, lys⁵⁰, lys⁷¹, lys⁸⁴, lys¹²¹, lys¹²², lys¹³⁴, lys¹³⁵, and lys¹⁶⁵ in a second CIFN polypeptide, where the first and second CIFN polypeptides are the same or different. A composition suitable for use in a subject method can further comprise a third monopegylated CIFN polypeptide species of molecules characterized by a PEG moiety linked to a second lysine residue selected from one of lys³¹, lys⁵⁰, lys⁷¹, lys⁸⁴, lys¹²¹, lys¹²², lys¹³⁴, lys¹³⁵, and lys¹⁶⁵ in a third CIFN polypeptide, where the third CIFN polypeptide is the same or different from either of the first and second CIFN polypeptides, where the second lysine residue is located in a position in the amino acid sequence of the third CIFN polypeptide that is not the same as the position of the first lysine residue in the amino acid sequence of the second CIFN polypeptide. A composition suitable for use in a subject method may further comprise at least one additional monopegylated CIFN polypeptide species of molecules characterized by a PEG moiety linked to one of lys³¹, lys⁵⁰, lys⁷¹, lys⁸⁴, lys¹²¹, lys¹²², lys¹³⁴, lys¹³⁵, and lys¹⁶⁵, where the location of the linkage site in each additional monopegylated CIFN polypeptide species is not the same as the location of the linkage site in any other species. In all species in this example, the PEG moiety is a linear PEG moiety having an average molecular weight of about 30 kD.

As another example, a composition suitable for use in a subject method comprises a population of monopegylated CIFN molecules, consisting of a first monopegylated CIFN polypeptide species of molecules characterized by a PEG moiety linked at the N-terminal amino acid residue of a first CIFN polypeptide, and a second monopegylated CIFN polypeptide species of molecules characterized by a PEG moiety linked to a first lysine residue selected from one of lys¹²¹, lys¹³⁴, lys¹³⁵, and lys¹⁶⁵ in a second CIFN polypeptide, where the first and second CIFN polypeptides are the same or different. A composition suitable for use in a subject method can further comprise a third monopegylated CIFN polypeptide species of molecules characterized by a PEG moiety linked to a second lysine residue selected from one of lys¹²¹, lys¹³⁴, lys¹³⁵, and lys¹⁶⁵ in a third CIFN polypeptide, where the third CIFN polypeptide is the same or different from either of the first and second CIFN polypeptides, where the second lysine residue is located in a position in the amino acid sequence of the third CIFN polypeptide that is not the same as the position of the first lysine residue in the amino acid sequence of the second CIFN polypeptide. A composition suitable for use in a subject method may further comprise at least one additional monopegylated CIFN polypeptide species of molecules characterized by a PEG moiety linked to one of lys¹²¹, lys¹³⁴, lys¹³⁵, and lys¹⁶⁵, where the location of the linkage site in each additional monopegylated CIFN polypeptide species is not the same as the location of the linkage site in any other species. In all species in this example, the PEG moiety is a linear PEG moiety having an average molecular weight of about 30 kD.

As another non-limiting example, a composition suitable for use in a subject method comprises a population of monopegylated CIFN molecules, consisting of a first monopegylated CIFN polypeptide species of molecules characterized by a PEG moiety linked to a first lysine residue in a first CIFN polypeptide; and a second monopegylated CIFN polypeptide species of molecules characterized by a PEG moiety linked at a second lysine residue in a second CIFN polypeptide, where the first and second CIFN polypeptides are the same or different, and where the first lysine is located in a position in the amino acid sequence of the first CIEN polypeptide that is not the same as the position of the second lysine residue in the amino acid sequence of the second CIFN polypeptide. A composition suitable for use in a subject method may further comprise at least one additional monopegylated CIFN species of molecules characterized by a PEG moiety linked to a lysine residue in the CIFN polypeptide, where the location of the linkage site in each additional monopegylated CIFN polypeptide species is not the same as the location of the linkage site in any other species. In all species in this example, the PEG moiety is a linear PEG moiety having an average molecular weight of about 30 kD.

As another non-limiting example, a composition suitable for use in a subject method comprises a population of monopegylated CIFN molecules, consisting of a first monopegylated CIFN polypeptide species of molecules characterized by a PEG moiety linked at a first lysine residue chosen from lys³¹, lys⁵⁰, lys⁷¹, lys⁸⁴, lys¹²¹, lys¹²², lys¹³⁴, lys¹³⁵, and lys¹⁶⁵ in a first CIFN polypeptide; and a second monopegylated CIFN polypeptide species of molecules characterized by a PEG moiety linked at a second lysine residue chosen from lys³¹, lys⁵⁰, lys⁷¹, lys⁸⁴, lys¹²¹, lys¹²², lys¹³⁴, lys¹³⁵, and lys¹⁶⁵ in a second CIFN polypeptide, where the first and second CIFN polypeptides are the same or different, and where the second lysine residue is located in a position in the amino acid sequence of the second CIFN polypeptide that is not the same as the position of the first lysine residue in the first CIFN polypeptide. The composition may further comprise at least one additional monopegylated CIFN polypeptide species of molecules characterized by a PEG moiety linked to one of lys³¹, lys⁵⁰, lys⁷¹, lys⁸⁴, lys¹²¹, lys¹²², lys¹³⁴, lys¹³⁵, and lys¹⁶⁵, where the location of the linkage site in each additional monopegylated CIFN polypeptide species is not the same as the location of the linkage site in any other species. In all species in this example, the PEG moiety is a linear PEG moiety having an average molecular weight of about 30 kD.

As another non-limiting example, a composition suitable for use in a subject method comprises a population of monopegylated CIFN molecules, consisting of a first monopegylated CIFN polypeptide species of molecules characterized by a PEG moiety linked at a first lysine residue chosen from lys¹²¹, lys¹³⁴, lys¹³⁵, and lys¹⁶⁵ in a first CIFN polypeptide; and a second monopegylated CIFN polypeptide species of molecules characterized by a PEG moiety linked at a second lysine residue chosen from lys¹²¹, lys¹³⁴, lys¹³⁵, and lys¹⁶⁵ in a second CIFN polypeptide, where the first and second CIFN polypeptides are the same or different, and where the second lysine residue is located in a position in the amino acid sequence of the second CIFN polypeptide that is not the same as the position of the first lysine residue in the first CIFN polypeptide. The composition may further comprise at least one additional monopegylated CIFN polypeptide species of molecules characterized by a PEG moiety linked to one of lys¹²¹, lys¹³⁴, lys¹³⁵, and lys¹⁶⁵, where the location of the linkage site in each additional monopegylated CIFN polypeptide species is not the same as the location of the linkage site in any other species. In all species in this example, the PEG moiety is a linear PEG moiety having an average molecular weight of about 30 kD.

As another non-limiting example, a composition suitable for use in a subject method comprises a monopegylated population of CIFN molecules, consisting of a first monopegylated CIFN polypeptide species of molecules characterized by a PEG moiety linked to a first surface-exposed lysine residue in a first CIFN polypeptide; and a second monopegylated CIFN polypeptide species of molecules characterized by a PEG moiety linked at a second surface-exposed lysine residue in a second CIFN polypeptide, where the first and second CIFN polypeptides are the same or different, and where the first surface-exposed lysine is located in a position in the amino acid sequence of the first CIFN polypeptide that is not the same as the position of the second surface-exposed lysine residue in the amino acid sequence of the second CIFN polypeptide. A composition suitable for use in a subject method may further comprise at least one additional monopegylated CIFN species of molecules characterized by a PEG moiety linked to a surface-exposed lysine residue in the CIFN polypeptide, where the location of the linkage site in each additional monopegylated CIFN polypeptide species is not the same as the location of the linkage site in any other species. In all species in this example, the PEG moiety is a linear PEG moiety having an average molecular weight of about 30 kD.

In connection with each of the above-described populations of monopegylated CIFN molecules, the invention contemplates embodiments where the molecules in each such population comprise a CIFN polypeptide chosen from interferon alpha-con₁, interferon alpha-con₂, and interferon alpha-con₃.

The invention further features use of a product that is produced by the process of reacting CIFN polypeptide with a succinimidyl ester of alpha-methoxy, omega-propionylpoly(ethylene glycol) (mPEGspa) that is linear and about 30 kD in molecular weight, where the reactants are initially present at a molar ratio of about 1:1 to about 1:5 CIFN:mPEGspa, and where the reaction is conducted at a pH of about 7 to about 9, followed by recovery of the monopegylated CIFN product of the reaction. In one embodiment, the reactants are initially present at a molar ratio of about 1:3 CIFN:mPEGspa and the reaction is conducted at a pH of about 8. In another embodiment where the product is generated by a scaled-up procedure needed for toxicological and clinical investigations, the reactants are initially present in a molar ratio of 1:2 CIFN:nPEGspa and the reaction is conducted at a pH of about 8.0.

In connection with the above-described product-by-process, the invention contemplates embodiments where the CIFN reactant is chosen from interferon alpha-con₁, interferon alpha-con₂, and interferon alpha-con₃.

IFN-β

The term interferon-beta (“IFN-β”) includes IFN-β polypeptides that are naturally occurring; non-naturally-occurring IFN-β polypeptides; and analogs of naturally occurring or non-naturally occurring IFN-β that retain antiviral activity of a parent naturally-occurring or non-naturally occurring IFN-β.

Any of a variety of beta interferons can be administered in a subject method. Suitable beta interferons include, but are not limited to, naturally-occurring IFN-β; IFN-β1a, e.g., Avonex® (Biogen, Inc.), and Rebif® (Serono, SA); IFN-β1b (Betaseron®; Berlex); and the like.

The IFN-β formulation may comprise an N-blocked species, wherein the N-terminal amino acid is acylated with an acyl group, such as a formyl group, an acetyl group, a malonyl group, and the like. Also suitable for use is a consensus IFN-β.

IFN-β polypeptides can be produced by any known method. DNA sequences encoding IFN-β may be synthesized using standard methods. In many embodiments, IFN-β polypeptides are the products of expression of manufactured DNA sequences transformed or transfected into bacterial hosts, e.g., E. coli, or in eulkaryotic host cells (e.g., yeast; mammalian cells, such as CHO cells; and the like). In these embodiments, the IFN-β is “recombinant IFN-β.” Where the host cell is a bacterial host cell, the IFN-β is modified to comprise an N-terminal methionine.

It is to be understood that IFN-β as described herein may comprise one or more modified amino acid residues, e.g., glycosylations, chemical modifications, and the like.

IFN-tau

The term interferon-tau includes IFN-tau polypeptides that are naturally occurring; non-naturally-occurring IFN-tau polypeptides; and analogs of naturally occurring or non-naturally occurring IFN-tau that retain antiviral activity of a parent naturally-occurring or non-naturally occurring IFN-tau.

Suitable tau interferons include, but are not limited to, naturally-occurring IFN-tau; Tauferon® (Pepgen Corp.); and the like.

IFN-tau may comprise an amino acid sequence as set forth in any one of GenBank Accession Nos. P15696; P56828; P56832; P56829; P56831; Q29429; Q28595; Q28594; S08072; Q08071; Q08070; Q08053; P56830; P28169; P28172; and P28171. The sequence of any known IFN-tau polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

The IFN-tau formulation may comprise an N-blocked species, wherein the N-terminal amino acid is acylated with an acyl group, such as a formyl group, an acetyl group, a malonyl group, and the like. Also suitable for use is a consensus IFN-tau.

IFN-tau polypeptides can be produced by any known method. DNA sequences encoding IFN-tau may be synthesized using standard methods. In many embodiments, IFN-tau polypeptides are the products of expression of manufactured DNA sequences transformed or transfected into bacterial hosts, e.g., E. coli, or in eulkaryotic host cells (e.g., yeast; mammalian cells, such as CHO cells; and the like). In these embodiments, the IFN-tau is “recombinant IFN-tau.” Where the host cell is a bacterial host cell, the IFN-tau is modified to comprise an N-terminal methionine.

It is to be understood that IFN-tau as described herein may comprise one or more modified amino acid residues, e.g., glycosylations, chemical modifications, and the like.

IFN-ω

The term interferon-omega (“IFN-ω”) includes IFN-ω polypeptides that are naturally occurring; non-naturally-occurring IFN-ω polypeptides; and analogs of naturally occurring or non-naturally occurring IFN-ω that retain antiviral activity of a parent naturally-occurring or non-naturally occurring IFN-ω.

Any known omega interferon can be administered in a subject method. Suitable IFN-ω include, but are not limited to, naturally-occurring IFN-ω; recombinant IFN-ω, e.g., Biomed 510 (BioMedicines); and the like.

IFN-ω may comprise an amino acid sequence as set forth in GenBank Accession No.

NP_{—}002168; or AAA70091. The sequence of any known IFN-ω polypeptide may be altered in various ways known in the art to generate targeted changes in sequence. A variant polypeptide will usually be substantially similar to the sequences provided herein, i. e. will differ by at least one amino acid, and may differ by at least two but not more than about ten amino acids. The sequence changes may be substitutions, insertions or deletions. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g, acetylation, or carboxylation; changes in amino acid sequence that introduce or remove a glycosylation site; changes in amino acid sequence that make the protein susceptible to PEGylation; and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

The IFN-ω formulation may comprise an N-blocked species, wherein the N-terminal amino acid is acylated with an acyl group, such as a formyl group, an acetyl group, a malonyl group, and the like. Also suitable for use is a consensus IFN-ω.

IFN-ω polypeptides can be produced by any known method. DNA sequences encoding IFN-ω may be synthesized using standard methods. In many embodiments, IFN-ω polypeptides are the products of expression of manufactured DNA sequences transformed or transfected into bacterial hosts, e.g., E. coli, or in eukaryotic host cells (e.g., yeast; mammalian cells, such as CHO cells; and the like). In these embodiments, the IFN-ω is “recombinant IFN-ω.” Where the host cell is a bacterial host cell, the IFN-ω is modified to comprise an N-terminal methionine.

It is to be understood that IFN-ω as described herein may comprise one or more modified amino acid residues, e.g., glycosylations, chemical modifications, and the like.

Type III Interferon Receptor Agonists

In any of the above-described methods, the interferon receptor agonist is in some embodiments an agonist of a Type III interferon receptor (e.g., “a Type III interferon agonist”). Type III interferon agonists include an IL-28b polypeptide; and IL-28a polypeptide; and IL-29 polypeptide; antibody specific for a Type III interferon receptor; and any other agonist of Type III interferon receptor, including non-polypeptide agonists.

IL-28A, IL-28B, and IL-29 (referred to herein collectively as “Type III interferons” or “Type III IFNs”) are described in Sheppard et al. (2003) Nature 4:63-68. Each polypeptide binds a heterodimeric receptor consisting of IL-10 receptor β chain and an IL-28 receptor α. Sheppard et al. (2003), supra. The amino acid sequences of IL-28A, IL-28B, and IL-29 are found under GenBank Accession Nos. NP_{—}742150, NP_{—}742151, and NP_{—}742152, respectively.

The amino acid sequence of a Type III IFN polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

Included in the subject invention is the use of polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al.

J. Biol. Chem. 275:23783-23789). Analogs may be used that include residues other than naturally occurring L-amino acids, e.g. D-amino acids or non-naturally occurring synthetic amino acids. The protein may be pegylated to enhance stability. The polypeptides may be fused to albumin.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

Immunomodulatory Agents

Immunomodulatory agents that are suitable for use in a subject combination therapy include, but are not limited to, Type II interferon receptor agonists (including IFN-γ); TNF antagonists; pirfenidone and pirfenidone analogs; and thymosin-α.

Type II Interferon Receptor Agonists

Type II interferon receptor agonists include any naturally occurring or non-naturally-occurring ligand of a human Type II interferon receptor that binds to and causes signal transduction via the receptor. Type II interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

A specific example of a Type II interferon receptor agonist is IFN-gamma (IFN-γ) and variants thereof. While the present invention exemplifies use of an IFN-gamma polypeptide, it will be readily apparent that any Type II interferon receptor agonist can be used in a subject method.

Interferon-Gamma

The nucleic acid sequences encoding IFN-gamma polypeptides may be accessed from public databases, e.g., Genbank, journal publications, and the like. While various mammalian IFN-gamma polypeptides are of interest, for the treatment of human disease, generally the human protein will be used. Human IFN-gamma coding sequence may be found in Genbank, accession numbers X13274; V00543; and NM_{—}000619. The corresponding genomic sequence may be found in Genbank, accession numbers J00219; M37265; and V00536. See, for example. Gray et al. (1982) Nature 295:501 (Genbank X13274); and Rinderluiecht et al. (1984) J.B.C. 259:6790.

IFN-γ1b (Actimmune®; human interferon) is a single-chain polypeptide of 140 amino acids. It is made recombinantly in E. coli and is unglycosylated (Rinderkliecht et al. 1984, J. Biol. Chem. 259:6790-6797). Recombinant IFN-gamma as discussed in U.S. Pat. No. 6,497,871 is also suitable for use herein.

The IFN-gamma to be used in a subject method may be any of natural IFN-gamma, recombinant IFN-gamma and the derivatives thereof so far as they have an IFN-γ activity, particularly human IFN-gamma activity. Human IFN-gamma exhibits the antiviral and anti-proliferative properties characteristic of the interferons, as well as a number of other immunomodulatory activities, as is known in the art. Although IFN-gamma is based on the sequences as provided above, the production of the protein and proteolytic processing can result in processing variants thereof. The unprocessed sequence provided by Gray et al., supra, consists of 166 amino acids (aa). Although the recombinant IFN-gamma produced in E. coli was originally believed to be 146 amino acids, (commencing at amino acid 20) it was subsequently found that native human IFN-gamma is cleaved after residue 23, to produce a 143 aa protein, or 144 aa if the terminal methionine is present, as required for expression in bacteria. During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression. The N-terminal methionine is an artifact encoded by the mRNA translational “start” signal AUG that, in the particular case of E. coli expression is not processed away. In other microbial systems or eukaryotic expression systems, methionine may be removed.

For use in the subject methods, any of the native IFN-gamma peptides, modifications and variants thereof, or a combination of one or more peptides may be used. IFN-gamma peptides of interest include fragments, and can be variously truncated at the carboxyl terminus relative to the full sequence. Such fragments continue to exhibit the characteristic properties of human gamma interferon, so long as amino acids 24 to about 149 (numbering from the residues of the unprocessed polypeptide) are present. Extraneous sequences can be substituted for the amino acid sequence following amino acid 155 without loss of activity. See, for example, U.S. Pat. No. 5,690,925. Native IFN-gamma moieties include molecules variously extending from amino acid residues 24-150; 24-151, 24-152; 24- 153, 24-155; and 24-157. Any of these variants, and other variants known in the art and having IFN-γ activity, may be used in the present methods.

The sequence of the IFN-γ polypeptide may be altered in various ways known in the art to generate targeted changes in sequence. A variant polypeptide will usually be substantially similar to the sequences provided herein, i.e., will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation,.or carboxylation; changes in amino acid sequence that introduce or remove a glycosylation site; changes in amino acid sequence that make the protein susceptible to PEGylation; and the like. IFN-gamma may be modified with one or more polyethylene glycol moieties (PEGylated). In one embodiment, the invention contemplates the use of IFN-gamma variants with one or more non-naturally occurring glycosylation and/or pegylation sites that are engineered to provide glycosyl- and/or PEG-derivatized polypeptides with reduced serum clearance, such as the IFN-gamma polypeptide variants described in any of International Patent Publication Nos. WO 01/36001 and WO 02/081507. Also included are modifications of glycosylation, e.g., those made by modifying the glycosylation patterns of a polypeptide during its synthesis and processing or in further processing steps; e.g., by exposing the polypeptide to enzymes that affect glycosylation, such as mammalian glycosylating or deglycosylating enzymes. Also embraced are sequences that have phosphorylated amino acid residues, e.g., phosphotyrosine, phosphoserine, or phosphothreonine.

Included in the subject invention is the use of IFN-γ polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see, for example, Friedler et al. 2000, J. Biol. Chem. 275:23783-23789). Analogs may be used that include residues other than naturally occurring L-amino acids, e.g., D-amino acids or non-naturally occurring synthetic amino acids. The protein may be pegylated to enhance stability.

The IFN-γ polypeptides may be prepared by in vitro synthesis, using conventional methods as known in the art, by recombinant methods, or may be isolated from cells induced or naturally producing the protein. The particular sequence and the method of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The IFN-γ polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

TNF Antagonists

Suitable TNF-α antagonists for use herein include agents that decrease the level of TNF-α synthesis, agents that block or inhibit the binding of TNF-α to a TNF-α receptor (TNFR), and agents that block or inhibit TNFR-mediated signal transduction. Unless otherwise expressly stated, every reference to a “TNF-α antagonist” or “TNF antagonist” herein will be understood to mean a TNF-α antagonist other than pirfenidone or a pirfenidone analog.

As used herein, the terms “TNF receptor polypeptide” and “TNFR polypeptide” refer to polypeptides derived from TNFR (from any species) which are capable of binding TNF. Two distinct cell-surface TNFRs have described: Type II TNFR (or p75 TNFR or TNFRII) and Type I TNFR (or p55 TNFR or TNFRI). The mature full-length human p75 TNFR is a glycoprotein having a molecular weight of about 75-80 kilodaltons (kD). The mature full-length human p55 TNFR is a glycoprotein having a molecular weight of about 55-60 kD. Exemplary TNFR polypeptides are derived from TNFR Type I and/or TNFR type II. Soluble TNFR includes p75 TNFR polypeptide; fusions of p75 TNFR with heterologous fusion partners, e.g., the Fc portion of an immunoglobulin.

TNFR polypeptide may be an intact TNFR or a suitable fragment of TNFR. U.S. Pat. No. 5,605,690 provides examples of TNFR polypeptides, including soluble TNFR polypeptides, appropriate for use in the present invention. In many embodiments, the TNFR polypeptide comprises an extracellular domain of TNFR. In some embodiments, the TNFR polypeptide is a fusion polypeptide comprising an extracellular domain of TNFR linked to a constant domain of an immunoglobulin molecule. In other embodiments, the TNFR polypeptide is a fusion polypeptide comprising an extracellular domain of the p75 TNFR linked to a constant domain of an IgG1 molecule. In some embodiments, when administration to humans is contemplated, an Ig used for fusion proteins is human, e.g., human IgG1.

Monovalent and multivalent forms of TNFR polypeptides may be used in the present invention. Multivalent forms of TNFR polypeptides possess more than one TNF binding site. In some embodiments, the TNFR is a bivalent, or dimeric, form of TNFR. For example, as described in U.S. Pat. No. 5,605,690 and in Mohler et al., 1993, J. Tumunol., 151:1548-1561, a chimeric antibody polypeptide with TNFR extracellular domains substituted for the variable domains of either or both of the immunoglobulin heavy or light chains would provide a TNFR polypeptide for the present invention. Generally, when such a chimeric TNFR:antibody polypeptide is produced by cells, it forms a bivalent molecule through disulfide linkages between the immunoglobulin domains. Such a chimeric TNFR:antibody polypeptide is referred to as TNFR:Fc.

In one embodiment, a subject method involves administration of an effective amount of the soluble TNFR ENBREL® etanercept. ENBREL® is a dimeric fusion protein consisting of the extracellular ligand-binding portion of the human 75 kilodalton (p75) TNFR linked to the Fe portion of human IgG1. The Fc component of ENBREL® contains the CH2 domain, the CH3 domain and hinge region, but not the CH1 domain of IgG1. ENBREL® is produced in a Chinese hamster ovary (CHO) mammalian cell expression system. It consists of 934 amino acids and has an apparent molecular weight of approximately 150 kilodaltons. Smith et al. (1990) Science 248:1019-1023; Mohler et al. (1993) J. Immunol. 151:1548-1561; U.S. Pat. No. 5,395,760; and U.S. Pat. No. 5,605,690.

Also suitable for use are monoclonal antibodies that bind TNF-α. Monoclonal antibodies include “humanized” mouse monoclonal antibodies; chimeric antibodies; monoclonal antibodies that are at least about 80%, at least about 90%, at least about 95%, or 100% human in amino acid sequence; and the like. See, e.g., WO 90/10077; WO 90/04036;

and WO 92/02190. Suitable monoclonal antibodies include antibody fragments, such as Fv, F(ab′)₂ and Fab; synthetic antibodies; artificial antibodies; phage display antibodies; and the like.

Examples of suitable monoclonal antibodies include infliximab (REMICADE®, Centocor); and adalimumab (HUMIRA™, Abbott). REMICADE® is a chimeric monoclonal anti-TNF-α antibody that includes about 25% mouse amino acid sequence and about 75% human amino acid sequence. REMICADE® comprises a variable region of a mouse monoclonal anti-TNF-α antibody fused to the constant region of a human IgG1. Elliott et al. (1993) Arthritis Rheum. 36:1681-1690; Elliott et al. (1994) Lancet 344:1105-1110; Baert et al. (1999) Gastroenterology 116:22-28. HUMIRA™ is a human, full-length IgG1 monoclonal antibody that was identified using phage display technology. Piascik (2003) J. Am. Pharm. Assoc. 43:327-328.

Also included in the term “TNF antagonist,” and therefore suitable for use in a subject method, are stress-activated protein kinase (SAPK) inhibitors. SAPK inhibitors are known in the art, and include, but are not limited to 2-alkyl imidazoles disclosed in U.S. Pat. No. 6,548,520; 1,4,5-substituted imidazole compounds disclosed in U.S. Pat. No. 6,489,325; 1,4,5-substituted imidazole compounds disclosed in U.S. Pat. No. 6,569,871; heteroaryl aminophenyl ketone compounds disclosed in Published U.S. Patent Application No. 2003/0073832; pyridyl imidazole compounds disclosed in U.S. Pat. No. 6,288,089; and heteroaryl aminobenzophenones disclosed in U.S. Pat. No. 6,432,962. Also of interest are compounds disclosed in U.S. Patent Application Publication No. 2003/0149041; and U.S. Pat. No. 6,214,854. A stress-activated protein kinase is a member of a family of nitrogen-activated protein kinases which are activated in response to stress stimuli. SAPK include, but are not limited to, p38 (Lee et al. (1994) Nature 372:739) and c-jun N-terminal kinase (JNK).

Methods to assess TNF antagonist activity are known in the art and exemplified herein. For example, TNF antagonist activity may be assessed with a cell-based competitive binding assay. In such an assay, radiolabeled TNF is mixed with serially diluted TNF antagonist and cells expressing cell membrane bound TNFR. Portions of the suspension are centrifuged to separate free and bound TNF and the amount of radioactivity in the free and bound fractions determined. TNF antagonist activity is assessed by inhibition of TNF binding to the cells in the presence of the TNF antagonist.

As another example, TNF antagonists may be analyzed for the ability to neutralize TNF activity in vitro in a bioassay using cells susceptible to the cytotoxic activity of TNF as target cells. In such an assay, target cells, cultured with TNF, are treated with varying amounts of TNF antagonist and subsequently are examined for cytolysis. TNF antagonist activity is assessed by a decrease in TNF-induced target cell cytolysis in the presence of the TNF antagonist.

Pirfenidone and Analogs Thereof

In some embodiments, the subject method provides an immunomodulatory agent that is pirfenidone (5-methyl-1-phenyl-2-(1H)-pyridone) or a pirfenidone analog.
Pirfenidone 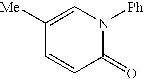
Pirfenidone Analogs 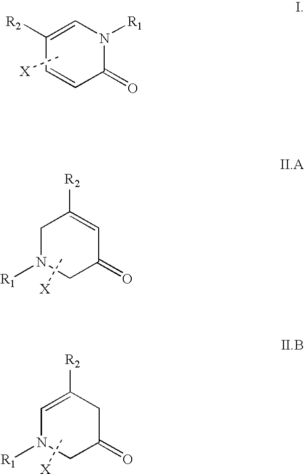
Descriptions for Substituents R₁, R₂, X

R₁: carbocyclic (saturated and unsaturated), heterocyclic (saturated or unsaturated), alkyls (saturated and unsaturated). Examples include phenyl, benzyl, pyrimidyl, naphthyl, indolyl, pyrrolyl, furyl, thienyl, imidazolyl, cyclohexyl, piperidyl, pyrrolidyl, morpholinyl, cyclohexenyl, butadienyl, and the like.

R₁ can further include substitutions on the carbocyclic or heterocyclic moieties with substituents such as halogen, nitro, amino, hydroxyl, alkoxy, carboxyl, cyano, thio, alkyl, aryl, heteroalkyl, heteroaryl and combinations thereof, for example, 4-nitrophenyl, 3-chliorophenyl, 2,5-dinitrophenyl, 4-metlioxyphenyl, 5-methyl-pyrrolyl, 2, 5-diclilorocyclohexyl, guanidinyl-cyclohexenyl and the like.

R₂: alkyl, carbocylic, aryl, heterocyclic. Examples include: methyl, ethyl, propyl, isopropyl, phenyl, 4-nitrophenyl, thienyl and the like.

X: may be any number (from 1 to 3) of substituents on the carbocyclic or heterocyclic ring. The substituents can be the same or different. Substituents can include hydrogen, alklyl, heteroalkyl, aryl, heteroaryl, halo, nitro, carboxyl, hydroxyl, cyano, amino, thio, alklylamino, haloaryl and the like.

The substituents may be optionally further substituted with 1-3 substituents from the group consisting of alkyl, aryl, nitro, alkoxy, hydroxyl and halo groups. Examples include: methyl, 2,3-dimethyl, phenyl, p-tolyl, 4-chlorophenyl, 4-nitrophenyl, 2,5-dichlorophenyl, furyl, thienyl and the like.

Specific Examples include those shown in Table 1:

TABLE 1

IA
5-Methyl-1-(2′-pyridyl)-2-(1H) pyridine,
6-Methyl-1-phenyl-2-(1H) pyridone,
5-Methyl-3-phenyl-1-(2′-thienyl)-2-(1H) pyridone,
5-Methyl-1-(2′-naphthyl)-2-(1H) pyridone,
5-Methyl-1-p-tolyl-2-(1H) pyridone,
5-Methyl-1-(1′naphthyl)-2-(1H) pyridone,
5-Ethyl-1-phenyl-2-(1H) pyridone,
5-Methyl-1-(5′-quinolyl)-2-(1H) pyridone,
5-Methyl-1-(4′-quinolyl)-2-(1H) pyridone,
5-Methyl-1-(4′-pyridyl)-2-(1H) pyridone,
3-Methyl-1-phenyl-2-(1H) pyridone,
5-Methyl-1-(4′-methoxyphenyl)-2-(1H) pyridone,
1-Phenyl-2-(1H) pyridone,
1,3-Diphenyl-2-(1H) pyridone,
1,3-Diphenyl-5-methyl-2-(1H) pyridone,
5-Methyl-1-(3′-trifluoromethylphenyl)-2-(1H)-pyridone,
3-Ethyl-1-phenyl-2-(1H) pyridone,
5-Methyl-1-(3′-pyridyl)-2-(1H) pyridone,
5-Methyl-1-(3-nitrophenyl)-2-(1H) pyridone,
3-(4′-Chlorophenyl)-5-Methyl-1-phenyl-2-(1H) pyridone,
5-Methyl-1-(2′-Thienyl)-2-(1H) pyridone,
5-Methyl-1-(2′-thiazolyl)-2-(1H) pyridone,
3,6-Dimethyl-1-phenyl-2-(1H) pyridone,
1-(4′Chlorophenyl)-5-Methyl-2-(1H) pyridone,
1-(2′-Imidazolyl)-5-Methyl-2-(1H) pyridone,
1-(4′-Nitrophenyl)-2-(1H) pyridone,
1-(2′-Furyl)-5-Methyl-2-(1H) pyridone,
1-Phenyl-3-(4′-chlorophenyl)-2-(1H) pyridine.
IIB
6-Methyl-1-phenyl-3-(1H) pyridone,
5-Methyl-1-p-tolyl-3-(1H) pyridone,
5-Methyl-1-(2′-naphthyl)-3-(1H) pyridone,
5-Methyl-1-phenyl-3-(1H) pyridone,
5-Methyl-1-(5′-quinolyl)-3-(1H) pyridone,
5-Ethyl-1-phenyl-3-(1H) pyridone,
5-Methyl-1-(4′-methoxyphenyl)-3-(1H) pyridone,
4-Methyl-1-phenyl-3-(1H) pyridone,
5-Methyl-1-(3′-pyridyl)-3-(1H) pyridone,
5-Methyl-1-(2′-Thienyl)-3-(1H) pyridone,
5-Methyl-1-(2′-pyridyl)-3-(1H) pyridone,
5-Methyl-1-(2′-quinolyl)-3-(1H) pyridone,
1-Phenyl-3-(1H) pyridine,
1-(2′-Furyl)-5-methyl-3-(1H) pyridone,
1-(4′-Chlorophenyl)-5-methyl-3-(1H) pyridine.

U.S. Pat. Nos. 3,974,281; 3,839,346; 4,042,699; 4,052,509; 5,310,562; 5,518,729; 5,716,632; and 6,090,822 describe methods for the synthesis and formulation of pirfenidone and pirfenidone analogs in pharmaceutical compositions suitable for use in the methods of the present invention.

Thymosin-α

Thymosin-α (Zadaxin™; available from SciClone Pharmaceuticals, Inc., San Mateo, Calif.) is a synthetic form of thymosin alpha 1, a hormone found naturally in the circulation and produced by the thymus gland. Thymosin-α increases activity of T cells and NK cells. Zadaxin™ formulated for subcutaneous injection is a purified sterile lyophilized preparation of chemically synthesized thymosin alpha 1 identical to human thymoisin alpha 1. Thymosin alpha 1 is an acetylated polypeptide with the following sequence: Ac-Ser-Asp-Ala-Ala-Val -Asp-Thr-Ser-Ser-Glu-lle-Thr-Thr-Lys-Asp-Leu-Lys-Glu-Lys-Lys-Glu -Val-Val-Glu-Glu-Ala-Glu-Asn-OH, and having a molecular weight of 3,108 daltons. The lyophilized preparation contains 1.6 mg synthetic thymoosin-α, 50 mg mannitol, and sodium phosphate buffer to adjust the pH to 6.8.

Ribavirin

Ribavirin, 1-β-ribofuranosyl-1H-1,2,4-triazole-3-carboxamide, is a nucleoside analog available from ICN Pharmaceuticals, Inc., Costa Mesa, Calif., and is described in the Merck Index, compound No. 8199, Eleventh Edition. Its manufacture and formulation is described in U.S. Pat. No. 4,211,771. The invention also contemplates use of derivatives of ribavirin (see, e.g., U.S. Pat. No. 6,277,830). The ribavirin may be administered orally in capsule or tablet form, or in the same or different administration form and in the same or different route as another therapeutic agent administered in a subject combination therapy. Of course, other types of administration of ribavirin, as they become available are contemplated, such as by nasal spray, transdermally, by suppository, by sustained release dosage form, etc. Any form of administration will work so long as the proper dosages are delivered without destroying the active ingredient.

Ribavirin is generally administered in an amount ranging from about 400 mg to about 1200 mg, from about 600 mg to about 1000 mg, or from about 700 to about 900 mg per day. In some embodiments, ribavirin is administered throughout the entire course of the combination therapy. In other embodiments, ribavirin is administered only during a first period of time. In still other embodiments, ribavirin is administered only during a second period of time.

Levovirin

Levovirin is the L-enantiomer of ribavirin, and exhibits the property of enhancing a Th1 immune response over a Th2 immune response. Levovirin is manufactured by ICN Pharmaceuticals.

Levovirin has the following structure: 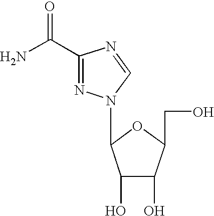
Viramidine

Viramidine is a 3-carboxamidine derivative of ribavirin, and acts as a prodrug of ribavirin. It is efficiently converted to ribavirin by adenosine deaminases.

Viramidine has the following structure: 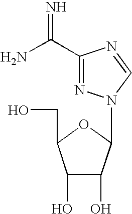
Nucleoside Analogs

Nucleoside analogs that are suitable for use in a subject combination therapy include, but are not limited to, ribavirin, levovirin, viramidine, isatoribine, an L-ribofuranosyl nucleoside as disclosed in U.S. Pat. No. 5,559,101 and encompassed by Formula I of U.S. Pat. No. 5,559,101 (e.g., 1-β-L-ribofuranosyluracil, 1-β-L-ribofuranosyl-5-fluorouracil, 1-β-L-ribofuranosylcytosine, 9-β-L-ribofuranosyladenine, 9-β-L-ribofuranosylhypoxanthine, 9-β-L-ribofuranosylguanine, 9-β-L-ribofuranosyl-6-thioguainie, 2-amino-α-L-ribofuranl[1′,2′:4,5]oxazoline, O²,O²-anhydro-1-α-L-ribofuranosyluracil, 1-α-L-ribofuranosyluracil, 1-(2,3,5-tri-O-benzoyl-α-ribofuranosyl)-4-thiouracil, 1-α-L-ribofuranosylcytosine, 1-α-L-ribofuranosyl-4-thiouracil, 1-α-L-ribofaranosyl-5-fluorouracil, 2-amino-β-L-arabinofurano [1′,2′:4,5]oxazoline, O²,O²-anhydro-β-L-arabinofuranosyluracil, 2′-deoxy-β-L-uridine, 3′5′-Di-O-benzoyl-2′deoxy-4-thio β-L-uridine, 2′-deoxy-β-L-cytidine, 2′-deoxy-β-L-4-thiouridine, 2′-deoxy-β-L-thymidine, 2′-deoxy-β-L-5-fluorouridine, 2′,3′-dideoxy-β-L-uridine, 2′-deoxy-β-L-5-fluorouridine, and 2′-deoxy-β-L-inosine); a compound as disclosed in U.S. Pat. No. 6,423,695 and encompassed by Formula I of U.S. Pat. No. 6,423,695; a compound as disclosed in U.S. Patent Publication No. 2002/0058635, and encompassed by Formula 1 of U.S. Patent Publication No. 2002/0058635; a nucleoside analog as disclosed in WO 01/90121 A2 (Idenix); a nucleoside analog as disclosed in WO 02/069903 A2 (Biocryst Pharmaceuticals Inc.); a nucleoside analog as disclosed in WO 02/057287 A2 or WO 02/057425 A2 (both MercklIsis); and the like.

HCV NS3 Inhibitors

Suitable HCV non-structural protein-3 (NS3) inhibitors include, but are not limited to, a tri-peptide as disclosed in U.S. Pat. Nos. 6,642,204, 6,534,523, 6,420,380, 6,410,531, 6,329,417, 6,329,379, and 6,323,180 (Boehringer-Ingelheim); a compound as disclosed in U.S. Pat. No. 6,143,715 (Boehringer-Ingelheim); a macrocyclic compound as disclosed in U.S. Pat. No. 6,608,027 (Boehringer-Ingelheim); an NS3 inhibitor as disclosed in U.S. Pat. Nos. 6,617,309, 6,608,067, and 6,265,380 (Vertex Pharmaceuticals); an azapeptide compound as disclosed in U.S. Pat. No. 6,624,290 (Schering); a compound as disclosed in U.S. Pat. No. 5,990,276 (Schering); a compound as disclosed in Pause et al. (2003) J. Biol. Chem. 278:20374-20380; NS3 inhibitor BILN 2061 (Boehringer-Ingelheim; Lamarre et al. (2002) Hepatology 36:301A; and Lamarre et al. (Oct. 26, 2003) Nature doi:10.1038/nature02099); NS3 inhibitor VX-950 (Vertex Pharmaceuticals; Kwong et al. (Oct. 24-28, 2003) 54^{th} Ann. Meeting AASLD); NS3 inhibitor SCH6 (Abib et al. (Oct. 24-28, 2003) Abstract 137. Program and Abstracts of the 54^{th} Annual Meeting of the American Association for the Study of Liver Diseases (AASLD). Oct. 24-28, 2003. Boston, Mass.); any of the NS3 protease inhibitors disclosed in WO 99/07733, WO 99/07734, WO 00/09558, WO 00/09543, WO 00/59929 or WO 02/060926 (e.g., compounds 2, 3, 5, 6, 8, 10, 11, 18, 19, 29, 30, 31, 32, 33, 37, 38, 55, 59, 71, 91, 103, 104, 105, 112, 113, 114, 115, 116, 120, 122, 123, 124, 125, 126 and 127 disclosed in the table of pages 224-226 in WO 02/060926); an NS3 protease inhibitor as disclosed in any one of U.S. Patent Publication Nos. 2003019067, 20030187018, and 20030186895; and the like.

Of particular interest in many embodiments are NS3 inhibitors that are specific NS3 inhibitors, e.g., NS3 inhibitors that inhibit NS3 serine protease activity and that do not show significant inhibitory activity against other serine proteases such as human leukocyte elastase, porcine pancreatic elastase, or bovine pancreatic chymotrypsin, or cysteine proteases such as human liver cathepsin B.

NS5B Inhibitors

Suitable HCV non-structural protein-5 (NS5; RNA-dependent RNA polymerase) inhibitors include, but are not limited to, a compound as disclosed in U.S. Pat. No. 6,479,508 (Boeliringer-Ingelheim); a compound as disclosed in any of International Patent Application Nos. PCT/CA02/01127, PCT/CA02/01128, and PCT/CA02/01129, all filed on Jul. 18, 2002 by Boehringer Ingelheim; a compound as disclosed in U.S. Pat. No. 6,440,985 (ViroPharma); a compound as disclosed in WO 01/47883, e.g., JTK-003 (Japan Tobacco); a dinucleotide analog as disclosed in Zhong et al. (2003) Antimicrob. Agents Chemother. 47:2674-268 1; a benzothiadiazine compound as disclosed in Dhanalc et al. (2002) J. Biol Chem. 277(41):38322-7; an NS5B inhibitor as disclosed in WO 02/100846 A1 or WO 02/100851 A2 (both Shire); an NS5B inhibitor as disclosed in WO 01/85172 A1 or WO 02/098424 A1 (both Glaxo SmithKline); an NS5B inhibitor as disclosed in WO 00/06529 or WO 02/06246 A1 (both Merck); an NS5B inhibitor as disclosed in WO 03/000254 (Japan Tobacco); an NS5B inhibitor as disclosed in EP 1 256,628 A2 (Agouron); JTK-002 (Japan Tobacco); JTK-109 (Japan Tobacco); and the like.

Of particular interest in many embodiments are NS5 inhibitors that are specific NS5 inhibitors, e.g., NS5 inhibitors that inhibit NS5 RNA-dependent RNA polymerase and that lack significant inhibitory toward other RNA dependent RNA polymerases and toward DNA dependent RNA polymerases.

Additional Antiviral Therapeutic Agents

Additional antiviral therapeutic agents that can be administered in a subject combination therapy include, but are not limited to, inhibitors of inosine monophosphate dehydrogenase (IMPDH); ribozymes that are complementary to viral nucleotide sequences; antisense RNA inhibitors; and the like.

IMPDH inhibitors

IMPDH inhibitors that are suitable for use in a subject combination therapy include, but are not limited to, VX-497 ((S)—N-3-[3-(3-methoxy-4-oxazol-5-yl-phenyl)-ureido]-benzyl-carbamic acid tetrahydrofuran-3-yl-ester); Vertex Pharmaceuticals; see, e.g., Marldand et al. (2000) Antimicrob. Agents Chemother. 44:859-866); ribavirin (ICN Pharmaceuticals);

levovirin (Ribapharm; see, e.g., Watson (2002) Curr Opin Investig Drugs 3(5):680-3); viramidine (Ribapharm); and the like.

Ribozyme and Antisense

Ribozyme and antisense antiviral agents that are suitable for use in a subject combination therapy include, but are not limited to, ISIS 14803 (ISIS Pharmaceuticals/Elan Corporation; see, e.g., Witherell (2001) Curr Opin Investig Drugs. 2(11):1523-9); Heptazyme™; and the like.

Side Effect Management Agents

In some embodiments, a subject therapy comprises administering a palliative agent (e.g., an agent that reduces patient discomfort caused by a therapeutic agent), or other agent for the avoidance, treatment, or reduction of a side effect of a therapeutic agent. Such agents are also referred to as “side effect management agents.”

Suitable side effect management agents include agents that are effective in pain management; agents that ameliorate gastrointestinal discomfort; analgesics, anti-inflammatories, antipsychotics, antineurotics, anxiolytics, and hematopoietic agents. In addition, the invention contemplates the use of any compound for palliative care of patients suffering from pain or any other side effect in the course of treatment with a subject therapy. Exemplary palliative agents include acetaminophen, ibuprofen, and other NSAIDs, H2 blockers, and antacids.

Analgesics that can be used to alleviate pain in the methods of the invention include non-narcotic analgesics such as non-steroidal anti-inflammatory drugs (NSAIDs) acetaminophen, salicylate, acetyl-salicylic acid (aspirin, diflunisal), ibuprofen, Motrin, Naprosyn, Nalfon, and Trilisate, indomethacin, glucametacine, acemetacin, sulindac, naproxen, piroxicam, diclofenac, benoxaprofen, ketoprofen, oxaprozin, etodolac, letorolac tromethamine, kcetorolac, nabumetone, and the like, and mixtures of two or more of the foregoing.

Other suitable analgesics include fentanyl, buprenorphine, codeine sulfate, morphine hydrochloride, codeine, hydromorphone (Dilaudid), levorphanol (Levo-Dromoran), methadone (Dolophine), morphine, oxycodone (in Percodan), and oxymorphone (Numorphan). Also suitable for use are benzodiazepines including, but not limited to, flurazepain (Dalmane), diazepain (Valium), and Versed, and the like.

Anti-Inflammatory Agents

Suitable anti-inflammatory agents include, but are not limited to, steroidal anti-inflammatory agents, and non-steroidal anti-inflammatory agents.

Suitable steroidal anti-inflammatory agents include, but are not limited to, hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, conisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures of two or more of the foregoing.

Suitable non-steroidal anti-inflammatory agents, include, but are not limited to, 1) the oxicams, such as piroxicam, isoxicam, tenoxicam, and sudoxicamn; 2) the salicylates, such as aspirin, disalcid, benorylate, trilisate, safapryn, solprin, diflunisal; and fendosal; 3) the acetic acid derivatives, such as diclofenac, fenclofenac, indomethacin, sulindac, tolmetin, isoxepac, furofenac, tiopinac, zidometacin, acematacin, fentiazac, zomepiract, clidanac, oxepinac, and felbinac; 4) the fenamates, such as mefenamic, meclofenamic, flufenamic, niflumic, and tolfenamic acids; 5) the propionic acid derivatives, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and 6) the pyrazoles, such as phenylbutazone, oxyphenbutazone, feprazone, azapropazone, and trimethazone, mixtures of these non-steroidal anti-inflammatory agents may also be employed, as well as the pharmaceutically-acceptable salts and esters of these agents.

Suitable anti-inflammatory agents include, but are not limited to, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; -Dexamethasone Dipropionate; Diclofenac Potassium; Diclofenac Sodium; Diflorasone Diacetate; −Diflumidone Sodium; Diflunisal; Difluprednate; Diftalone; Dimethyl Sulfoxide; Drocinonide; Endrysone; Enlimomab Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

Antipsychotic and antineurotic drugs that can be used to alleviate psychiatric side effects in the methods of the invention include any and all selective serotonin receptor inhibitors (SSRIs) and other anti-depressants, anxiolytics (e.g. alprazolam), etc. Anti-depressants include, but are not limited to, serotonin reuptake inhibitors such as Celexa®, Desyrel®, Effexor®, Luvox®, Paxil®, Prozac®, Zoloft®, and Serzone®; tricyclics such as Adapin®, Anafrinil®, Elavil®, Janimmine®, Ludiomil®, Pamelor®, Tofranil®, Vivactil®, Sinequan®, and Surmontil®; monoamine oxidase inhibitors such as Eldepryl®, Marplan®, Nardil®, and Parnate®. Anti-anxiety agents include, but are not limited to, azaspirones such as BuSpar®, benzodiazepines such as Ativan®, Librium®, Tranxene®, Centrax®, Klonopin®, Paxipam®, Serax®, Valium®, and Xanax®; and beta-blockers such as Inderal® and Tenormin®.

Agents that reduce gastrointestinal discomfort such as nausea, diarrhea, gastrointestinal cramping, and the like are suitable palliative agents for use in a subject combination therapy. Suitable agents include, but are not limited to, antiemetics, anti-diarrheal agents, H2 blockers, antacids, and the like.

Suitable H2 blockers (histamine type 2 receptor antagonists) that are suitable for use as a palliative agent in a subject therapy include, but are not limited to, Cimetidine (e.g., Tagamet, Peptol, Nu-cimet, apo-cimetidine, non-cimetidine); Ranitidine (e.g., Zantac, Nu-ranit, Novo-randine, and apo-ranitidine); and Famotidine (Pepcid, Apo-Famotidine, and Novo-Famotidine).

Suitable antacids include, but are not limited to, aluminum and magnesium hydroxide (Maalox®, Mylanta®; aluminum carbonate gel (Basajel®; aluminum hydroxide (Amphojel®, AlternaGEL®); calcium carbonate (Tums®, Titralac®); magnesium hydroxide; and sodium bicarbonate.

Antiemetics include, but are not limited to, 5-hydroxytryptophan-3 (5HT3) inhibitors; corticosteroids such as dexamethasone and methylprednisolone; Marinol® (dronabinol); prochlolperazine; benzodiazepines; promethazine; and metoclopramide cisapride; Alosetron Hydrochloride; Batanopride Hydrochloride; Bemesetron; Benzquinamide; Chlorpromazine; Chlorpromazine Hydrochloride; Clebopride; Cyclizine Hydrochloride; Dimenhydrinate; Diphenidol; Diphenidol Hydrochloride; Diphenidol Pamoate; Dolasetron Mesylate; Domperidone; Dronabinol; Fludorex; Flumeridone; Galdansetron Hydrochloride; Granisetron; Granisetron Hydrochloride; Lurosetron Mesylate; Meclizine Hydrochloride; Metoclopramide Hydrochloride; Metopimazine; Ondansetron Hydrochloride; Pancopride; Prochlorperazine; Prochlorperazine Edisylate; Prochlorperazine Maleate; Prometliazine Hydrochloride; Thiethylperazine; Thiethylperazine Malate; Thiethylperazine Maleate; Trimethobenzamide Hydrochloride; Zacopride Hydrochloride.

Anti-diarrheal agents include, but are not limited to, Rolgamidine, Diphenoxylate hydrochloride (Lomotil), Metronidazole (Flagyl), Methylprednisolone (Medrol), Sulfasalazine (Azulfidine), and the like.

Suitable hematopoietic agents that can be used to prevent or restore depressed blood cell populations in the methods of the invention include erythropoietins, such as EPOGEN™ epoetin-alfa, granulocyte colony stimulating factors (G-CSFs), such as NEUPOGEN™ filgrastim, granulocyte-macrophage colony stimulating factors (GM-CSFs), thrombopoietins, etc.

Dosages, Formulations, and Route of Administration

A therapeutic agent (also referred to as an “active agent”) used in a subject method, e.g., i) a Type I interferon receptor agonist or a Type III interferon receptor agonist; ii) an immunomodulatory agent; iii) an inhibitor of an HCV enzyme, is administered to individuals in a formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20^{th} edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In the subject methods, the active agents (e.g., (i) a Type I interferon receptor agonist or a Type III interferon receptor agonist; ii) an immunomodulatory agent; iii) an inhibitor of an HCV enzyme; and optionally one or more additional active agents) may be administered to the host using any convenient means capable of resulting in the desired therapeutic effect. Thus, the agents can be incorporated into a variety of formulations for therapeutic administration. More particularly, and active agent (e.g., (i) a Type I interferon receptor agonist or a Type III interferon receptor agonist; ii) an immunomodulatory agent; iii) an inhibitor of an HCV enzyme; and optionally one or more additional active agents) can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of an active agent (e.g., (i) a Type I interferon receptor agonist or a Type III interferon receptor agonist; ii) an immunomodulatory agent; iii) an inhibitor of an HCV enzyme; and optionally one or more additional active agents) can be achieved in various ways, including oral, buccal, rectal, parenteral, intraperitoneal, intradermal, subcutaneous, intramuscular, transdermal, intratracheal, etc., administration. In some embodiments, two different routes of administration are used. For example, in some embodiments, IFN-α is administered subcutaneously, and pirfenidone is administered orally.

Subcutaneous administration of an active agent (e.g., (i) a Type I interferon receptor agonist or a Type III interferon receptor agonist; ii) an immunomodulatory agent; iii) an inhibitor of an HCV enzyme; and optionally one or more additional active agents) can be accomplished using standard methods and devices, e.g., needle and syringe, a subcutaneous injection port delivery system, and the like. See, e.g., U.S. Pat. Nos. 3,547,119; 4,755,173;

4,531,937; 4,311,137; and 6,017,328. A combination of a subcutaneous injection port and a device for administration of a therapeutic agent to a patient through the port is referred to herein as “a subcutaneous injection port delivery system.” In some embodiments, subcutaneous administration is achieved by a combination of devices, e.g., bolus delivery by needle and syringe, followed by delivery using a continuous delivery system.

In some embodiments, a therapeutic agent, e.g., i) a Type I interferon receptor agonist or a Type III interferon receptor agonist; ii) an immunomodulatory agent; iii) an inhibitor of an HCV enzyme, is delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, the present methods of drug delivery can be accomplished using any of a variety of refillable, pump systems. Pumps provide consistent, controlled release over time. Typically, the agent is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. All implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are generally preferred because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electronmechanical infusion pump can also be suitable for use with the present invention. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems will in some embodiments be used, due to their generally more consistent, controlled release over time. Osmotic pumps are particularly preferred due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, a therapeutic agent (e.g., (i) a Type I interferon receptor agonist or a Type III interferon receptor agonist; ii) an immunomodulatory agent; iii) an inhibitor of an HCV enzyme; and optionally one or more additional active agents) is delivered using an implantable drug delivery system, e.g., a system that is programmable to provide for administration of a therapeutic agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the Synchromed infusion pump (Medtronic).

In pharmaceutical dosage forms, the active agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the active agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The active agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the active agents can be made into suppositories by mixing with a variety of bases such as emulsifying bases or water-soluble bases. An active agent can can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent calculated in an amount sufficient to produce the desired effect in association with a pharmaceutically acceptable diluent, carrier or vehicle. The specifications for a particular active agent depend on the particular agent employed and the effect to be achieved, and the pharmacodynamics associated with each agent in the host.

In connection with each of the methods described herein, the invention provides embodiments in which the therapeutic agent(s) is/are administered to the patient by a controlled drug delivery device. In some embodiments, the therapeutic agent(s) is/are delivered to the patient substantially continuously or continuously by the controlled drug delivery device. Optionally, an implantable infusion pump is used to deliver the therapeutic agent(s) to the patient substantially continuously or continuously by subcutaneous infusion.

In other embodiments, a therapeutic agent is administered to the patient so as to achieve and maintain a desired average daily serum concentration of the therapeutic agent at a substantially steady state for the duration of the monotherapy or combination therapy. Optionally, an implantable infusion pump is used to deliver the therapeutic agent to the patient by subcutaneous infusion so as to achieve and maintain a desired average daily serum concentration of the therapeutic agent at a substantially steady state for the duration of the therapeutic agent in monotherapy or combination therapy.

Type I or Type III interferon receptor agonist

In some embodiments, the Type I or III interferon receptor agonist is an IFN-α. Effective dosages of an IFN-α can range from about 1 μg to about 30 μg, from about 3 μg to about 27 μg, from about 1 MU to about 20 MU, from about 3 MU to about 10 MU, from about 90 μg to about 180 μg, or from about 18 μg to about 90 μg.

Effective dosages of Infergen consensus IFN-α include about 3 μg, about 9 μg, about 15 μg, about 18 μg, or about 27 μg of drug per dose. Effective dosages of IFN-α2a and IFN-α2b can range from 3 million Units (MU) to 10 MU per dose. Effective dosages of PEGylated IFN-α2a can contain an amount of about 90 μg to 180 μg, or about 135 μg, of drug per dose. Effective dosages of PEGylated IFN-α2b can contain an amount of about 0.5 μg to 1.5 μg of drug per kg of body weight per dose. Effective dosages of PEGylated consensus interferon (PEG-CIFN) can contain an amount of about 10 μg to about 100 μg, or about 18 μg to about 90 μg, or about 27 μg to about 60 μg, or about 45 μg, of CIFN amino acid weight per dose of PEG-CIFN. IFN-α can be administered daily, every other day, once a week, three times a week, every other week, three times per month, once monthly, substantially continuously or continuously.

In some embodiments, monoPEG (30 kD, linear)-ylated consensus IFN-α is administered. In some embodiments, monoPEG (30 kD, linear)-ylated consensus IFN-α is administered at a dosing interval of every 7 days. In some embodiments, monoPEG (30 kD, linear)-ylated consensus IFN-α is administered at a dosing interval of every 8 days to every 14 days, e.g., once every 8 days, once every 9 days, once every 10 days, once every 11 days, once every 12 days, once every 13 days, or once every 14 days, or at a dosing interval greater than 14 days. Effective dosages of monoPEG (30 kD, linear)-ylated INFERGEN® consensus IFN-α generally range from about 45 μg to about 270 μg per dose, e.g., 60 μg per dose, 100 μg per dose, 150 μg per dose, 200 μg per dose, etc.

In some embodiments, a Type I or III interferon receptor agonist is administered in a first dosing regimen, followed by a second dosing regimen. The first dosing regimen of Type I or III interferon receptor agonist (also referred to as “the induction regimen ”) generally involves administration of a higher dosage of the Type I or III interferon receptor agonist. For example, in the case of INFERGEN® consensus IFN-α (CIFN), the first dosing regimen comprises administering CIFN at about 9 μg, about 15 μg, about 18 μg, or about 27 μg. The first dosing regimen can encompass a single dosing event, or at least two or more dosing events. The first dosing regimen of the Type I or III interferon receptor agonist can be administered daily, every other day, three times a week, every other week, three times per month, once monthly, substantially continuously or continuously.

The first dosing regimen of the Type I or III interferon receptor agonist is administered for a first period of time, which time period can be at least about 4 weeks, at least about 8 weeks, or at least about 12 weeks.

The second dosing regimen of the Type I or III interferon receptor agonist (also referred to as “the maintenance dose”) generally involves administration of a lower amount of the Type I or III interferon receptor agonist. For example, in the case of CIFN, the second dosing regimen comprises administering CIFN at least about 3 μg, at least about 9 μg, at least about 15 μg, or at least about 18 μg. The second dosing regimen can encompass a single dosing event, or at least two or more dosing events.

The second dosing regimen of the Type I or III interferon receptor agonist can be administered daily, every other day, three times a week, every other week, three times per month, once monthly, substantially continuously or continuously.

In some embodiments, where an “induction“/“maintenance” dosing regimen of a Type I or a III interferon receptor agonist is administered, a “priming” dose of a Type II interferon receptor agonist is included. In these embodiments, Type II interferon receptor agonist can be administered for a period of time from about 1 day to about 14 days, from about 2 days to about 10 days, or from about 3 days to about 7 days, before the beginning of treatment with the Type I or III interferon receptor agonist. This period of time is referred to as the “priming” phase. In some of these embodiments, Type II interferon receptor agonist treatment is continued throughout the entire period of treatment with the Type I or III interferon receptor agonist. In other embodiments, Type II interferon receptor agonist treatment is discontinued before the end of treatment with the Type I or III interferon receptor agonist. In some of these embodiments, the total time of treatment with the Type II interferon receptor agonist (including the “priming” phase) is from about 2 days to about 30 days, from about 4 days to about 25 days, from about 8 days to about 20 days, from about 10 days to about 18 days, or from about 12 days to about 16 days.

In other embodiments, the Type I or III interferon receptor agonist is administered in a non-induction (single) dosing regimen. For example, in the case of CIFN, the dose of CIFN is generally in a range of from about 3 μg to about 15 μg, or from about 9 μg to about 15 μg. The dose of Type I or a Type III interferon receptor agonist is generally administered daily, every other day, three times a week, every other week, three times per month, once monthly, or substantially continuously. The dose of the Type I or III interferon receptor agonist is administered for a period of time, which period can be, for example, from at least about 24 weeks to at least about 48 weeks, or longer.

In some embodiments, where a single dosing regimen of a Type I or III interferon receptor agonist is administered, a “priming” dose of Type II interferon receptor agonist is included. For example, a Type II interferon receptor agonist can be administered for a period of time from about 1 day to about 14 days, from about 2 days to about 10 days, or from about 3 days to about 7 days, before the beginning of treatment with the Type I or III interferon receptor agonist. This period of time is referred to as the “priming” phase. In some of these embodiments, Type II interferon receptor agonist treatment is continued throughout the entire period of treatment with the Type I or III interferon receptor agonist. In other embodiments, Type II interferon receptor agonist treatment is discontinued before the end of treatment with Type I or III interferon receptor agonist. In some of these embodiments, the total time of treatment with the Type II interferon receptor agonist (including the “priming” phase) is from about 2 days to about 30 days, from about 4 days to about 25 days, from about 8 days to about 20 days, from about 10 days to about 18 days, or from about 12 days to about 16 days. Type II interferon receptor agonist

In some embodiments, the Type II interferon receptor agonist is an IFN-γ. Effective dosages of IFN-γ can range from about 0.5 μg/m² to about 500 μg/m², usually from about 1.5 μg/m² to 200 μg/m², depending on the size of the patient. This activity is based on 10⁶ international units (U) per 50 μg of protein. IFN-γ can be administered daily, every other day, three times a week, or substantially continuously or continuously.

In specific embodiments of interest, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-γ per body weight (assuming a range of body weights of from about 45 kg to about 135 kg) is in the range of from about 4.4 μg IFN-γ per kg body weight to about 1.48 μg IFN-γ per kg body weight.

The body surface area of subject individuals generally ranges from about 1.33 m² to about 2.50 m². Thus, in many embodiments, an IFN-γ dosage ranges from about 150 μg/m² to about 20 μg/m². For example, an IFN-γ dosage ranges from about 20 μg/m² to about 30 μg/m², from about 30 μg/m² to about 40 μg/m², from about 40 μg/m² to about 50 μg/m², from about 50 μg/m² to about 60 μg/m², from about 60 μg/m² to about 70 μg/m², from about 70 μg/m² to about 80 μg/m², from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m². In some embodiments, the dosage groups range from about 25 μg/m² to about 100 μg/m². In other embodiments, the dosage groups range from about 25 μg/m² to about 50 μg/m².

TNF Antagonist

Effective dosages of a TNF-α antagonist range from 0.1 μg to 40 mg per dose, e.g., from about 0.1 μg to about 0.5 μg per dose, from about 0.5 μg to about 1.0 μg per dose, from about 1.0 μg per dose to about 5.0 μg per dose, from about 5.0 μg to about 10 μg per dose, from about 10 μg to about 20 μg per dose, from about 20 μg per dose to about 30 μg per dose, from about 30 μg per dose to about 40 μg per dose, from about 40 μg per dose to about 50 μg per dose, from about 50 μg per dose to about 60 μg per dose, from about 60 μg per dose to about 70 μg per dose, from about 70 μg to about 80 μg per dose, from about 80 μg per dose to about 100 μg per dose, from about 100 μg to about 150 μg per dose, from about 150 μg to about 200 μg per dose, from about 200 μg per dose to about 250 μg per dose, from about 250 μg to about 300 μg per dose, from about 300 μg to about 400 μg per dose, from about 400 μg to about 500 μg per dose, from about 500 μg to about 600 μg per dose, from about 600 μg to about 700 μg per dose, from about 700 μg to about 800 μg per dose, from about 800 μg to about 900 μg per dose, from about 900 μg to about 1000 μg per dose, from about 1 mg to about 10 mg per dose, from about 10 mg to about 15 mg per dose, from about 15 mg to about 20 mg per dose, from about 20 mg to about 25 mg per dose, from about 25 mg to about 30 mg per dose, from about 30 mg to about 35 mg per dose, or from about 35 mg to about 40 mg per dose.

In some embodiments, the TNF-α antagonist is EMBREL® etanercept. Effective dosages of etanercept range from about 0.1 μg to about 40 mg per dose, from about 0.1 μg to about 1 μg per dose, from about 1 μg to about 10 μg per dose, from about 10 μg to about 100 μg per dose, from about 100 μg to about 1 mg per dose, from about 1 mg to about 5 mg per dose, from about 5 mg to about 10 mg, from about 10 mg to about 15 mg per dose, from about 15 mg to about 20 mg per dose, from about 20 mg to about 25 mg per dose, from about 25 mg to about 30 mg per dose, from about 30 mg to about 35 mg per dose, or from about 35 mg to about 40 mg per dose.

In some embodiments, effective dosages of a TNF-α antagonist are expressed as mg/kg body weight. In these embodiments, effective dosages of a TNF-α antagonist are from about 0.1 mg/kg body weight to about 10 mg/kg body weight, e.g., from about 0.1 mg/kg body weight to about 0.5 mg/kg body weight, from about 0.5 mg/kg body weight to about 1.0 mg/kg body weight, from about 1.0 mg/kg body weight to about 2.5 mg/kg body weight, from about 2.5 mg/kg body weight to about 5.0 mg/kg body weight, from about 5.0 mg/kg body weight to about 7.5 mg/kg body weight, or from about 7.5 mg/kg body weight to about 10 mg/kg body weight.

In some embodiments, the TNF-α antagonist is REMICADE® infliximab. Effective dosages of REMICADE® range from about 0.1 mg/kg to about 10 mg/kg, from about 0.1 mg/kg to about 0.5 mg/kg, from about 0.5 mg/kg to about 1.0 mg/kg, from about 1.0 mg/kg to about 1.5 mg/kg, from about 1.5 mg/kg to about 2.0 mg/kg, from about 2.0 mg/kg to about 2.5 mg/kg, from about 2.5 mg/kg to about 3.0 mg/kg, from about 3.0 mg/kg to about 3.5 mg/kg, from about 3.5 mg/kg to about 4.0 mg/kg, from about 4.0 mg/kg to about 4.5 mg/kg, from about 4.5 mg/kg to about 5.0 mg/kg, from about 5.0 mg/kg to about 7.5 mg/kg, or from about 7.5 mg/kg to about 10 mg/kg per dose.

In some embodiments the TNF-α antagonist is HUMIRA™ adalimumab. Effective dosages of HUMIRA™ range from about 0.1 μg to about 35 mg, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, or from about 35 mg to about 40 mg per dose.

In many embodiments, a TNF-α antagonist is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The TNF-α antagonist can be administered tid, bid, qd, qod, biw, tiw, qw, qow, three times per month, once monthly, substantially continuously, or continuously.

In many embodiments, multiple doses of a TNF-α antagonist are administered. For example, a TNF-α antagonist is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Those of skill in the art will readily appreciate that dose levels can vary as a function of the specific compounds, the severity of the symptoms and the susceptibility of the subject to side effects. Preferred dosages for a given compound are readily determinable by those of skill in the art by a variety of means. A preferred means is to measure the physiological potency of a given compound.

Pirfenidone or a Pirfenidone Analog

Pirfenidone or a pirfenidone analog can be administered once per month, twice per month, three times per month, once per week, twice per week, three times per week, four times per week, five times per week, six times per week, daily, or in divided daily doses ranging from once daily to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Effective dosages of pirfenidone or a specific pirfenidone analog include a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally. Other doses and formulations of pirfenidone and specific pirfenidone analogs suitable for use are described in U.S. Pat. Nos., 5,310,562; 5,518,729; 5,716,632; and 6,090,822.

Thyrnosin-α

Thymosin-α (Zadaxin™) is generally administered by subcutaneous injection. Thymosin-α can be administered tid, bid, qd, qod, biw, tiw, qw, qow, three times per month, once monthly, substantially continuously, or continuously. In many embodiments, thymosin-α is administered twice per week.

Effective dosages of thymosin-α range from about 0.5 mg to about 5 mg, e.g., from about 0.5 mg to about 1.0 mg, from about 1.0 mg to about 1.5 mg, from about 1.5 mg to about 2.0 mg, from about 2.0 mg to about 2.5 mg, from about 2.5 mg to about 3.0 mg, from about 3.0 mg to about 3.5 mg, from about 3.5 mg to about 4.0 mg, from about 4.0 mg to about 4.5 mg, or from about 4.5 mg to about 5.0 mg. In particular embodiments, thymosin-α is administered in dosages containing an amount of 1.0 mg or 1.6 mg.

Thymosin-α can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Ribavirin, Levovirin, Viramidine

Ribavirin is generally administered in an amount ranging from about 30 mg to about 60 mg, from about 60 mg to about 125 mg, from about 125 mg to about 200 mg, from about 200 mg to about 300 μm, from about 300 mg to about 400 mg, from about 400 mg to about 1200 mg, from about 600 mg to about 1000 mg, or from about 700 to about 900 mg per day, or about 10 mg/kg body weight per day. In some embodiments, ribavirin is administered orally in dosages of about 400, about 800, about 1000, or about 1200 mg per day.

Levovirin is generally administered in an amount ranging from about 30 mg to about 60 mg, from about 60 mg to about 125 mg, from about 125 mg to about 200 mg, from about 200 mg to about 300 μm, from about 300 mg to about 400 mg, from about 400 mg to about 1200 mg, from about 600 mg to about 1000 mg, or from about 700 to about 900 mg per day, or about 10 mg/kg body weight per day. In some embodiments, levovirin is administered orally in dosages of about 400, about 800, about 1000, or about 1200 mg per day.

Viramidine is generally administered in an amount ranging from about 30 mg to about 60 mg, from about 60 mg to about 125 mg, from about 125 mg to about 200 mg, from about 200 mg to about 300 μm, from about 300 mg to about 400 mg, from about 400 mg to about 1200 mg, from about 600 mg to about 1000 mg, or from about 700 to about 900 mg per day, or about 10 mg/kg body weight per day. In some embodiments, viramidine is administered orally in dosages of about 800, or about 1600 mg per day.

In many embodiments, multiple doses of a ribavirin, levovirin, viramidine, isatoribine, and/or other nucleoside analogs are administered. For example, a nucleoside analog is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Those of skill in the art will readily appreciate that dose levels can vary as a function of the specific compounds, the severity of the symptoms and the susceptibility of the subject to side effects. Preferred dosages for a given compound are readily determinable by those of skill in the art by a variety of means. A preferred means is to measure the physiological potency of a given compound.

NS3 Inhibitors, NS5B Inhibitors

Effective dosages of an HCV enzyme inhibitor range from about 10 mg to about 200 mg per dose, e.g., from about 10 mg to about 15 mg per dose, from about 15 mg to about 20 mg per dose, from about 20 mg to about 25 mg per dose, from about 25 mg to about 30 mg per dose, from about 30 mg to about 35 mg per dose, from about 35 mg to about 40 mg per dose, from about 40 mg per dose to about 45 mg per dose, from about 45 mg per dose to about 50 mg per dose, from about 50 mg per dose to about 60 mg per dose, from about 60 mg per dose to about 70 mg per dose, from about 70 mg per dose to about 80 mg per dose, from about 80 mg per dose to about 90 mg per dose, from about 90 mg per dose to about 100 mg per dose, from about 100 mg per dose to about 125 mg per dose, from about 125 mg per dose to about 150 mg per dose, from about 150 mg per dose to about 175 mg per dose, or from about 175 mg per dose to about 200 mg per dose.

In some embodiments, effective dosages of an HCV enzyme inhibitor are expressed as mg/kg body weight. In these embodiments, effective dosages of an HCV enzyme inhibitor are from about 0.01 mg/kg body weight to about 100 mg/kg body weight, from about 0.I mg/kg body weight to about 50 mg/kg body weight, from about 0.1 mg/kg body weight to about 1 mg/kg body weight, from about 1 mg/kg body weight to about 10 mg/kg body weigh, from about 10 mg/kg body weight to about 100 mg/kg body weight, from about 5 mg/kg body weight to about 400 mg/kg body weight, from about 5 mg/kg body weight to about 50 mg/kg body weight, from about 50 mg/kg body weight to about 100 mg/kg body weight, from about 100 mg/kg body weight to about 200 mg/kg body weight, from about 200 mg/kg body weight to about 300 mg/kg body weight, or from about 300 mg/kg body weight to about 400 mg/kg body weight.

In many embodiments, an HCV enzyme inhibitor is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The HCV enzyme inhibitor can be administered tid, bid, qd, qod, biw, tiw, qw, qow, three times per month, once monthly, substantially continuously, or continuously.

In many embodiments, multiple doses of an HCV enzyme inhibitor are administered. For example, an HCV enzyme inhibitor is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Those of skill in the art will readily appreciate that dose levels can vary as a function of the specific compounds, the severity of the symptoms and the susceptibility of the subject to side effects. Preferred dosages for a given compound are readily determinable by those of skill in the art by a variety of means. A preferred means is to measure the physiological potency of a given compound.

Side Effect Management Agent

Any subject monotherapy or combination therapy can be modified to include administration of a side effect management agent. Side effects of Type I interferon receptor agonist treatment include, but are not limited to, fever, malaise, tachycardia, chills, headache, arthralgia, myalgia, myelosuppression, suicide ideation, platelet suppression, neutropenia, lymphocytopenia, erythrocytopenia (anemia), and anorexia. In some embodiments, an effective amount of a palliative agent reduces a side effect induced by treatment with a Type I interferon receptor agonist by at least about 10%, at least about 20%, at least about 30%, at least about 40%, at least about 50%, at least about 60%, or more, compared to the rate of occurrence or the degree or extent of the side effect when the Type I interferon receptor agonist is administered without the palliative agent. For example, if a fever is experienced with the Type I interferon receptor agonist therapy, then the body temperature of an individual treated with the Type I interferon receptor agonist therapy and palliative agent according to the instant invention is reduced by at least 0.5 degree Fahrenheit, and in some embodiments is within the normal range, e.g., at or near 98.6° F.

Side effects of pirfenidone or a pirfenidone analog include gastrointestinal disturbances and discomfort. Gastrointestinal disturbances include nausea, diarrhea, gastrointestinal cramping, and the like. In some embodiments, an effective amount of a palliative agent reduces a side effect induced by treatment with a pirfenidone or a pirfenidone analog by at least about 10%, at least about 20%, at least about 30%, at least about 40%, at least about 50%, at least about 60%, or more, compared to the rate of occurrence or the degree or extent of the side effect when the pirfenidone or pirfenidone analog is administered without the palliative agent.

Side effects of other, additional therapeutic agents (e.g., anti-angiogenic agents; anti-cancer agents such as anti-proliferative agents, anti-neoplastic agents, and cytotoxic agents; anti-fibrotic agents; TNF-α antagonists; and anti-inflammatory agents) are well known. For example, side effects of anti-neoplastic agents include gastrointestinal discomfort. Other side effects of additional therapeutic agents include fever, malaise, etc.

Combination Regimens for Treating a Hepatitis C Virus Infection

The present invention provides methods of treating HCV infection by administering a combination of a Type I or III interferon receptor agonist, an immunomodulatory agent, and an HCV enzyme inhibitor in a therapeutically effective amount to an individual in need thereof. In some embodiments, the Type I interferon receptor agonist is IFN-α. Immunomodulatory agents can be selected from i) a Type II interferon receptor agonist; ii) a TNF antagonist; iii) pirfenidone or a pirfenidone analog; iv) and thymosin-α. Where the immunomodulatory agent is a Type II interferon receptor agonist, in many embodiments the Type II interferon receptor agonist is IFN-γ. HCV enzyme inhibitors are selected from NS3 protease inhibitors; NS3 lielicase inhibitors; and NS5B RNA-dependent RNA polymerase inhibitors. IFN-α, IFN-γ, and HCVenzyine inhibitor in combination therapy

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of an IFN-α selected from (i) INFERGEN( containing an amount of about 1 μg to about 30 μg of drug per dose of INFERGEN®, subcutaneously qd, qod, tiw, biw, qw, qow, three times per month, once monthly, or per day continuously or substantially continuously (ii) PEGylated consensus IFN-α (PEG-CIFN) containing an amount of about 10 μg to about 100 μg, or about 45 μg to about 60 μg, of CIFN amino acid weight per dose of PEG-CIFN subcutaneously qw, qow, three times per month, or monthly (iii) IFN-α2a, 2b or 2c containing an amount of about 3 MU to about 10 MU of drug per dose of IFN-α2a, 2b or 2c subcutaneously qd, qod, tiw, biw, or per day continuously or substantially continuously (iv) PEGASYS® containing an amount of about 90 μg to about 360 μg, or about 180 μg, of drug per dose of PEGASYS®, subcutaneously qw, qow, three times per month, or monthly (v) PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly or (vi) mono PEG(30 kD, linear)-ylated consensus IFN-α containing an amount of from about 100 μg to about 200 μg, or about 150 μg, of drug per dose of mono PEG(30 kD, linear)-ylated consensus IFN-α subcutaneously qw, qow, once every 8 days to once every 14 days, three times per month, or monthly; b) a dosage of IFN-γ containing an amount of from about 10 μg to about 300 μg of drug per dose of IFN-γ, subcutaneously qd, qod, tiw, or biw, or per day substantially continuously or continuously; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of INFERGEN® containing an amount of about 1 μg to about 30 μg of drug per dose of INFERGEN®, subcutaneously qd, qod, tiw, biw, qw, qow, three times per month, or once monthly, or per day continuously or substantially continuously; b) a dosage of IFN-γ containing an amount of from about 10 μg to about 300 μg of drug per dose of IFN-γ, subcutaneously qd, qod, tiw, or biw, or per day substantially continuously or continuously; and c) a dosage of an HCV NS3 protease inhibitor containing an amount of from about 0.01 mg to about 100 mg of drug per Idlogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGylated consensus IFN-α (PEG-CIFN) containing an amount of about 10 μg to about 100 μg, or about 45 μg to about 60 μg, of CIFN amino acid weight per dose of PEG-CIFN subcutaneously qw, qow, three times per month, or monthly; b) a dosage of IFN-γ containing an amount of from about 10 μg to about 300 μg of drug per dose of IFN-γ, subcutaneously qd, qod, tiw, or biw, or per day substantially continuously or continuously; and c) a dosage of an HCV NS3 protease inhibitor containing an amount of from about 0.01 mg to about 100 mg of drug per Idlogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of IFN-α2a, 2b or 2c containing an amount of about 3 MU to about 10 MU of drug per dose of IFN-α2a, 2b or 2c subcutaneously qd, qod, tiw, biw, or per day continuously or substantially continuously; b) a dosage of IFN-γ containing an amount of from about 10 μg to about 300 μg of drug per dose of IFN-γ, subcutaneously qd, qod, tiw, or biw, or per day substantially continuously or continuously; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGASYS® peginterferon alfa-2a containing an amount of about 90 μg to about 360 μg, or about 180 μg, of drug per dose of PEGASYS®, subcutaneously qw, qow, three times per month, or monthly; b) a dosage of IFN-γ containing an amount of from about 10 μg to about 300 μg of drug per dose of IFN-γ, subcutaneously qd, qod, tiw, or biw, or per day substantially continuously or continuously; and c) a dosage of an HCV NS3 protease inhibitor containing an amount of from about 0.01 mg to about 100 mg of drug per kilogram body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEG-INTRON® peginterferon alfa-2b containing an amount of about 0.75 μg to about 3.0 μg, or about 1.0 μg, of drug per kilogram of body weight per dose of PEG-INTRON®, subcutaneously qw, qow, three times per month, or monthly; b) a dosage of IFN-γ containing an amount of from about 10 μg to about 300 μg of drug per dose of IFN-γ, subcutaneously qd, qod, tiw, or biw, or per day substantially continuously or continuously; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of mono PEG(30 kd, linear)-ylated consensus IFN-α containing an amount of from about 100 μg to about 200 μg, or about 150 μg, of drug per dose of mono PEG(30 kD, linear.)-ylated consensus IFN-α, subcutaneously qw, qow, once every 8 days to once every 14 days, three times per month, or once monthly; b) a dosage of IFN-γ containing an amount of from about 10 μg to about 300 μg of drug per dose of IFN-γ, subcutaneously qd, qod, tiw, or biw, or per day substantially continuously or continuously; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of an IFN-α selected from (i) INFERGEN® containing an amount of about 1 μg to about 30 μg of drug per dose of INFERGEN®, subcutaneously qd, qod, tiw, biw, qw, qow, three times per month, once monthly, or per day continuously or substantially continuously (ii) PEGylated consensus IFN-α (PEG-CIFN) containing an amount of about 10 μg to about 100 μg, or about 45 μg to about 60 μg, of CIFN amino acid weight per dose of PEG-CIFN subcutaneously qw, qow, three times per month, or monthly (iii) IFN-α2a, 2b or 2c containing an amount of about 3 MU to about 10 MU of drug per dose of IFN-α2a, 2b or 2c subcutaneously qd, qod, tiw, biw, or per day continuously or substantially continuously (iv) PEGASYS® containing an amount of about 90 μg to about 360 μg, or about 180 μg, of drug per dose of PEGASYS®, subcutaneously qw, qow, three times per month, or monthly (v) PEG-INTRON®D containing an amount of about 0.75 μg to about 3.0 μg, or about 1.0 μg, of drug per lilogram of body weight per dose of PEG-INTRON®, subcutaneously biw, qw, qow, three times per month, or monthly or (vi) mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly; b) a dosage of IFN-γ containing an amount of from about 10 μg to about 300 μg of drug per dose of IFN-γ, subcutaneously qd, qod, tiw, or biw, or per day substantially continuously or continuously; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of INFERGEN® containing an amount of about 1 μg to about 30 μg of drug per dose of INFERGEN®, subcutaneously qd, qod, tiw, biw, qw, qow, three times per month, once monthly, or per day continuously or substantially continuously; b) a dosage of IFN-γ containing an amount of from about 10 μg to about 300 μg of drug per dose of IFN-γ, subcutaneously qd, qod, tiw, or biw, or per day substantially continuously or continuously; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGylated consensus IFN-α (PEG-CIFN) containing an amount of about 10 μg to about 100 μg, or about 45 μg to about 60 μg, of CIFN amino acid weight per dose of PEG-CIFN, subcutaneously qw, qow, three times per month, or monthly; b) a dosage of IFN-γ containing an amount of from about 10 μg to about 300 μg of drug per dose of IFN-γ, subcutaneously qd, qod, tiw, or biw, or per day substantially continuously or continuously; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of IFN-α2 containing an amount of about 3 MU to about 10 MU of drug per dose of IFN-α2a, 2b or 2c, subcutaneously qd, qod, tiw, biw, or per day continuously or substantially continuously; b) a dosage of IFN-γ containing an amount of from about 10 μg to about 300 μg of drug per dose of IFN-γ, subcutaneously qd, qod, tiw, or biw, or per day substantially continuously or continuously; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGASYS® peginterferon alfa-2a containing an amount of about 90 μg to about 360 μg, or about 180 μg, of drug per dose of PEGASYS®, subcutaneously qw, qow, three times per month, or monthly; b) a dosage of IFN-γ containing an amount of from about 10 μg to about 300 μg of drug per dose of IFN-γ, subcutaneously qd, qod, tiw, or biw, or per day substantially continuously or continuously; and c) a dosage of an HCV NS5B RNA-dependent RNA polymerase inhibitor containing an amount of from about 0.01 mg to about 100 mg of drug per kilogram body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEG-INTRON® peginterferon alfa-2b containing an amount of about 0.75 μg to about 3.0 μg, or about 1.0 μg, of drug per kilogram of body weight per dose of PEG-INTRON®, subcutaneously biw, qw, qow, three times per month, or monthly; b) a dosage of IFN-γ containing an amount of from about 10 μg to about 300 μg of drug per dose of IFN-γ, subcutaneously qd, qod, tiw, or biw, or per day substantially continuously or continuously; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of mono PEG(30 kD, linear)-ylated consensus IFN-α containing an amount of from about 100 μg to about 200 μg, or 150 μg, of drug per dose of mono PEG(30 kD, linear)-ylated consensus IFN-α, subcutaneously qw, qow, once every 8 days to once every 14 days, three times per month, or once monthly; b) a dosage of IFN-γ containing an amount of from about 10 μg to about 300 μg of drug per dose of IFN-γ, subcutaneously qd, qod, tiw, or biw, or per day substantially continuously or continuously; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of an IFN-α selected from (i) INFERGEN® containing an amount of about 1 μg to about 30 μg of drug per dose of INFERGEN®, subcutaneously qd, qod, tiw, biw, qw, qow, three times per month, once monthly, or per day continuously or substantially continuously (ii) PEGylated consensus IFN-α (PEG-CIFN) containing an amount of about 10 μg to about 100 μg, or about 45 μg to about 60 μg, of CIFN amino acid weight per dose of PEG-CIFN subcutaneously qw, qow, three times per month, or monthly (iii) IFN-α2a, 2b or 2c containing an amount of about 3 MU to about 10 MU of drug per dose of IFN-α2a, 2b or 2c subcutaneously qd, qod, tiw, biw, or per day continuously or substantially continuously (iv) PEGASYS® containing an amount of about 90 μg to about 360 μg, or about 180 μg, of drug per dose of PEGASYS®, subcutaneously qw, qow, three times per month, or monthly (v) PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly or (vi) mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly; b) a dosage of IFN-γ containing an amount of from about 10 μg to about 300 μg of drug per dose of IFN-γ, subcutaneously qd, qod, tiw, or biw, or per day substantially continuously or continuously; c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly; and d) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of INFERGEN® containing an amount of about 1 μg to about 30 μg of drug per dose of INFERGEN®, subcutaneously qd, qod, tiw, biw, qw, qow, three times per month, once monthly, or per day continuously or substantially continuously; b) a dosage of IFN-γ containing an amount of from about 10 μg to about 300 μg of drug per dose of IFN-γ, subcutaneously qd, qod, tiw, or biw, or per day substantially continuously or continuously; c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly; and d) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGylated consensus IFN-α (PEG-CIFN) containing an amount of about 10 μg to about 100 μg, or about 45 μg to about 60 μg, of CIFN amino acid weight per dose of PEG-CIFN, subcutaneously qw, qow, three times per month, or monthly; b) a dosage of IFN-γ containing an amount of from about 10 μg to about 300 μg of drug per dose of IFN-γ, subcutaneously qd, qod, tiw, or biw, or per day substantially continuously or continuously; c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly; and d) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of IFN-α2a, 2b or 2c containing an amount of about 3 MU to about 1 0 MU of drug per dose of IFN-α2a, 2b or 2c, subcutaneously qd, qod, tiw, biw, or per day continuously or substantially continuously; b) a dosage of IFN-γ containing an amount of from about 10 μg to about 300 μg of drug per dose of IFN-γ, subcutaneously qd, qod, tiw, or biw, or per day substantially continuously or continuously; c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly; and d) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGASYS® peginterferon alfa-2a containing an amount of about 90 μg to about 360 μg, or about 180 μg, of drug per dose of PEGASYS®, subcutaneously qw, qow, three times per month, or monthly; b) a dosage of IFN-γ containing an amount of from about 10 μg to about 300 μg of drug per dose of IFN-γ, subcutaneously qd, qod, tiw, or biw, or per day substantially continuously or continuously; c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly; and d) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEG-INTRON® peginterferon alfa-2b containing an amount of about 0.75 μg to about 3.0 μg, or about 1.0 μg, of drug per kilogram of body weight per dose of PEG-INTRON®, subcutaneously biw, qw, qow, three times per month, or monthly; b) a dosage of IFN-γ containing an amount of from about 10 μg to about 300 μg of drug per dose of IFN-γ, subcutaneously qd, qod, tiw, or biw, or per day substantially continuously or continuously; c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly; and d) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly; for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or once monthly; b) a dosage of IFN-γ containing an amount of from about 10 μg to about 300 μg of drug per dose of IFN-γ, subcutaneously qd, qod, tiw, or biw, or per day substantially continuously or continuously; c) a dosage of an HCV NS3 protease inhibitor containing an amount of from about 0.0 1 mg to about 100 mg of drug per kilogram of body weight per dose, orally tid, bid, qd, qod, tiw, biw, qw, qow, three times per month, or once monthly; and d) a dosage of an HCV NS5B RNA-dependent RNA polymerase inhibitor containing an amount of from about 0.01 mg to about 1 00 mg of drug per kilogram of body weight per dose, orally tid, bid, qd, qod, tiw, biw, qw, qow, three times per month, or once monthly; for the desired treatment duration, to achieve a sustained viral response.

Any of the above-described regimens involving administration of IFN-α, IFN-γ, and an NS3 protease inhibitor; IFN-α, IFN-γ, and an NS5B RNA-dependent RNA polymerase inhibitor; or IFN-α, IFN-γ, an NS3 protease inhibitor and an NS5B RNA-dependent RNA polymerase inhibitor, can be modified to further comprise administering an effective amount of an additional antiviral agent. In some embodiments, a subject combination therapy method for treating an HCV infection in an individual comprises co-administering effective amounts of IFN-α, IFN-γ, an NS3 protease inhibitor, and a second immunomodulatory, agent. In other embodiments, a subject combination therapy method for treating an HCV infection in an individual comprises co-administering effective amounts of IFN-α, IFN-γ, an NS5B RNA-dependent RNA polymerase inhibitor, and a second immunomodulatory agent. In other embodiments, a subject combination therapy method for treating an HCV infection in an individual comprises co-administering effective amounts of IFN-α, IFN-γ, an NS3 protease inhibitor, an NS5B RNA-dependent RNA polymerase inhibitor, and a second immunomodulatory agent.

As non-limiting examples, any of the above-described IFN-α, IFN-γ and HCV enzyme inhibitor combination regimens can be modified to include: (a) administering a dosage of a TNF-A antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; (b) administering a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; or (c) administering a dosage of Zadaxin™ thymosin-α containing an amount of 1.0 mg or 1.6 mg, administered subcutaneously twice per week for the desired treatment duration. IFN-α, TNF antagonist, and HCV enzyme inhibitor in combination therapy

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of an IFN-α selected from (i) INFERGEN® containing an amount of about 1 μg to about 30 μg of drug per dose of INFERGEN®, subcutaneously qd, qod, tiw, biw, qw, qow, three times per month, once monthly, or per day continuously or substantially continuously (ii) PEGylated consensus IFN-α (PEG-CIFN) containing an amount of about 10 μg to about 100 μg, or about 45 μg to about 60 μg, of CIFN amino acid weight per dose of PEG-CIFN subcutaneously qw, qow, three times per month, or monthly (iii) IFN-α2a, 2b or 2c containing an amount of about 3 MU to about 10 MU of drug per dose of IFN-α2a, 2b or 2c subcutaneously qd, qod, tiw, biw, or per day continuously or substantially continuously (iv) PEGASYS® containing an amount of about 90 μg to about 360 μg, or 180 μg, of drug per dose of PEGASYS®, subcutaneously qw, qow, three times per month, or monthly (v) PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly or (vi) mono PEG(30 kD), linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; and c) a dosage of an HCV NS3 protease inhibitor containing an amount of from about 0.01 mg to about 100 mg of drug per idlogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of INFERGEN® containing an amount of about 1 μg to about 30 μg of drug per dose of INFERGEN®, subcutaneously qd, qod, tiw, biw, qw, qow, three times per month, once monthly, or per day continuously or substantially continuously, for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; and c) a dosage of an HCV NS3 protease inhibitor containing an amount of from about 0.01 mg to about 100 mg of drug per kilogram body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGylated consensus IFN-α (PEG-CIFN) containing an amount of about 10 μg to about 100 μg, or about 45 μg to about 60 μg, of CIFN amino acid weight per dose of PEG-CIFN, subcutaneously qw, qow, three times per month, or monthly, for the desired treatment duration; b) a dosage of a TNF-A antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of IFN-α2a, 2b or 2c containing an amount of about 3 MU to about 10 MU of drug per dose of IFN-α2a, 2b or 2c, subcutaneously qd, qod, tiw, biw, or per day continuously or substantially continuously, for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGASYS® containing an amount of about 90 μg to about 360 μg, or about 180 μg, of drug per dose of PEGASYS®, subcutaneously qw, qow, three times per month, or monthly for the desired treatment duration; b) a dosage of a TNF-α antagonist selected from (i) ENBREL® in an amount of about 25 mg of drug subcutaneously biw (ii) REMICADE® in an amount of about 3 mg/kg to about 1 0 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; and c) a dosage of an HCV NS3 protease inhibitor containing an amount of from about 0.0 1 mg to about 1 00 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly, for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; and c) a dosage of an HCV NS3 protease inhibitor containing an amount of from about 0.0 1 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of an IFN-α selected from (i) INFERGEN® containing an amount of about 1 μg to about 30 μg of drug per dose of INFERGEN®, subcutaneously qd, qod, tiw, biw, qw, qow, three times per month, once monthly, or per day continuously or substantially continuously (ii) PEGylated consensus IFN-α (PEG-CIFN) containing an amount of about 10 μg to about 100 μg, or about 45 μg to about 60 μg, of CIFN amino acid weight per dose of PEG-CIFN subcutaneously qw, qow, three times per month, or monthly (iii) IFN-α2a, 2b or 2c containing an amount of about 3 MU to about 10 MU of drug per dose of IFN-α2a, 2b or 2c subcutaneously qd, qod, tiw, biw, or per day continuously or substantially continuously (iv) PEGASYS® containing an amount of about 90 μg to about 360 μg, or about 180 μg, of drug per dose of PEGASYS®, subcutaneously qw, qow, three times per month, or monthly (v) PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly or (vi) mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of a TNF-α antagonist selected from (i) ENBREL® in an amount of about 25 mg of drug subcutaneously biw (ii) REMICADE®D in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of INFERGEN® containing an amount of about 1 μg to about 30 μg of drug per dose of INFERGEN®, subcutaneously qd, qod, tiw, biw, qw, qow, three times per month, once monthly, or per day continuously or substantially continuously, for the desired treatment duration; b) a dosage of a TNF-A antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGylated consensus IFN-α (PEG-CIFN) containing an amount of about 10 μg to about 100 μg, or about 45 μg to about 60 μg, of CIFN amino acid weight per dose of PEG-CIFN subcutaneously qw, qow, three times per month, or monthly, for the desired treatment duration; b) a dosage of a TNF-A antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of IFN-α2a, 2b or 2c containing an amount of about 3 MU to about 1 0 MU of drug per dose of IFN-α2a, 2b or 2c, subcutaneously qd, qod, tiw, biw, or per day continuously or substantially continuously, for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGASYS® containing an amount of about 90 μg to about 360 μg, or about 180 μg, of drug per dose of PEGASYS®, subcutaneously qw, qow, three times per month, or monthly, for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly, for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of a TNF-α antagonist selected from (i) ENBREL® in an amount of about 25 mg of drug subcutaneously biw (ii) REMICADE®D in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; and c) a dosage of an HCV NS5B RNA-dependent RNA polymerase inhibitor containing an amount of from about 0.01 mg to about 1 00 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of an IFN-α selected from (i) INFERGEN® containing an amount of about 1 μg to about 30 μg of drug per dose of INFERGEN®, subcutaneously qd, qod, tiw, biw, qw, qow, three times per month, once monthly, or per day continuously or substantially continuously (ii) PEGylated consensus IFN-α (PEG-CIFN) containing an amount of about 10 μg to about 100 μg, or about 45 μg to about 60 μg, of CIFN amino acid weight per dose of PEG-CIFN subcutaneously qw, qow, three times per month, or monthly (iii) IFN-α2a, 2b or 2c containing an amount of about 3 MU to about 10 MU of drug per dose of IFN-α2a, 2b or 2c subcutaneously qd, qod, tiw, biw, or per day continuously or substantially continuously (iv) PEGASYS® containing an amount of about 90 μg to about 360 μg, or about 180 μg, of drug per dose of PEGASYS®, subcutaneously qw, qow, three times per month, or monthly (v) PEG-INTRON®M containing an amount of about 0.75 μg to about 3.0 μg, or about 1.0 μg, of drug per kilogram of body weight per dose of PEG-INTRON®, subcutaneously qw, qow, three times per month, or monthly or (vi) mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of INFERGEN® containing an amount of about 1 μg to about 30 μg of drug per dose of INFERGEN®, subcutaneously qd, qod, tiw, biw, qw, qow, three times per month, once monthly, or per day continuously or substantially continuously, for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGylated consensus IFN-α (PEG-CIFN) containing an amount of about 10 μg to about 100 μg, or about 45 μg to about 60 μg, of CIFN amino acid weight per dose of PEG-CIFN, subcutaneously qw, qow, three times per month, or monthly for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 46 mg of drug subcutaneously qw, qow, three times per month, once monthly, once every 6 weeks, or once every 8 weeks, for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of IFN-α2a, 2b or 2c containing an amount of about 3 MU to about 10 MU of drug per dose of IFN-α2a, 2b or 2c, subcutaneously qd, qod, tiw, biw, or per day continuously or substantially continuously, for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGASYS® containing an amount of about 90 μg to about 360 μg, or 180 μg, of drug per dose of PEGASYS®, subcutaneously qw, qow, three times per month, or monthly for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

Any of the above-described regimens involving administration of IFN-α, a TNF antagonist, and an NS3 protease inhibitor; IFN-α, a TNF antagonist, and an NS5B RNA-dependent RNA polymerase inhibitor; or IFN-α, a TNF antagonist, an NS3 protease inhibitor and an NS5B RNA-dependent RNA polymerase inhibitor, can be modified to further comprise administering an effective amount of an additional immunomodulatory agent. Thus, in some embodiments, a subject combination therapy method for treating an HCV infection in an individual comprises co-administering effective amounts of IFN-α, a TNF antagonist, an NS3 protease inhibitor, and a second immunomodulatory agent. In other embodiments, a subject combination therapy method for treating an HCV infection in an individual comprises co-administering effective amounts of IFN-α, a TNF antagonist, an NS5B RNA-dependent RNA polymerase inhibitor, and a second immunomodulatory agent. In other embodiments, a subject combination therapy method for treating an HCV infection in an individual comprises co-administering effective amounts of IFN-α, a TNF antagonist, an NS3 protease inhibitor, an NS5B RNA-dependent RNA polymerase inhibitor, and a second immunomodulatory agent.

As non-limiting examples, any of the above-described IFN-α, TNF antagonist and HCV enzyme inhibitor combination regimens can be modified to include: (a) administering a dosage of IFN-γ containing an amount of from about 10 μg to about 300 μg of drug per dose of IFN-γ, subcutaneously qd, qod, tiw, or biw, or per day substantially continuously or continuously; (b) administering a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; (c) a dosage of Zadaxin™ containing an amount of 1.0 mg or 1.6 mg, administered subcutaneously twice per week for the desired treatment duration; (d) a dosage of ribavirin or a derivative thereof, in an amount of about 400 mg, 800 mg, 1000 mg, or 1200 mg orally daily for the desired treatment duration; (e) a dosage of levovirin, in an amount of about 400 mg, 600 mg, 800 mg, 1000 mg, or 1200 mg orally daily for the desired treatment duration; or (f) a dosage of Viramidine™ in an amount of from about 800 mg to about 1600 mg orally daily for the desired treatment duration.

IFN-α, Pirfenidone or a Pirfenidone Analog, and an HCV Enzyme Inhibitor in Combination Therapy

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of an IFN-α selected from (i) INFERGEN® containing an amount of about 1 μg to about 30 μg of drug per dose of INFERGEN®, subcutaneously qd, qod, tiw, biw, qw, qow, three times per month, once monthly, or per day continuously or substantially continuously (ii) PEGylated consensus IFN-α (PEG-CIFN) containing an amount of about 10 μg to about 100 μg, or about 45 μg to about 60 μg, of CIFN amino acid weight per dose of PEG-CIFN subcutaneously qw, qow, three times per month, or monthly (iii) IFN-α2a, 2b or 2c containing an amount of about 3 MU to about 10 MU of drug per dose of IFN-α2a, 2b or 2c subcutaneously qd, qod, tiw, biw, or per day continuously or substantially continuously (iv) PEGASYS® containing an amount of about 90 μg to about 360 μg, or about 180 μg, of drug per dose of PEGASYS®, subcutaneously qw, qow, three times per month, or monthly (v) PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly or (vi) mono PEG(30 kD, linear)-ylated consensus IFN-α containing an amount of from about 100 μg to about 200 μg, or about 150 μg, of drug per dose of mono PEG(30 1kD, linear)-ylated consensus IFN-α, subcutaneously qw, qow, once every 8 days to once every 14 days, three times per month, or monthly, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of INFERGEN® containing an amount of about 1 μg to about 30 μg of drug per dose of INFERGEN® subcutaneously qd, qod, tiw, biw, qw, qow, three times per month, once monthly, or per day continuously or substantially continuously, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGylated consensus IFN-α (PEG-CIFN) containing an amount of about 10 μg to about 100 μg, or about 45 μg to about 60 μg, of CIFN amino acid weight per dose of PEG-CIFN, subcutaneously qw, qow, three times per month, or monthly, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of IFN-α2a, 2b or 2c containing an amount of about 3 MU to about 10 MU of drug per dose of IFN-α2a, 2b or 2c, subcutaneously qd, qod, tiw, biw, or per day continuously or substantially continuously, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGASYS® containing an amount of about 90 μg to about 360 μg, or about 180 μg, of drug per dose of PEGASYS®, subcutaneously qw, qow, three times per month, or monthly, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of mono PEG(30 kD, linear)-ylated consensus IFN-α containing an amount of from about 100 μg to about 200 μg, or about 150 μg, of drug per dose of mono PEG(30 1kD, linear)-ylated consensus IFN-α, subcutaneously qw, qow, once every 8 days to once every 14 days, three times per month, or monthly, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of an IFN-α selected from (i) INFERGEN® containing an amount of about 1 μg to about 30 μg of drug per dose of INFERGEN®, subcutaneously qd, qod, tiw, biw, qw, qow, three times per month, once monthly, or per day continuously or substantially continuously (ii) PEGylated consensus IFN-α (PEG-CIFN) containing an amount of about 10 μg to about 100 μg, or about 45 μg to about 60 μg, of CIFN amino acid weight per dose of PEG-CIFN subcutaneously qw, qow, three times per month, or monthly (iii) IFN-α2a, 2b or 2c containing an amount of about 3 MU to about 10 MU of drug per dose of IFN-α2a, 2b or 2c subcutaneously qd, qod, tiw, biw, or per day continuously or substantially continuously (iv) PEGASYS® containing an amount of about 90 μg to about 360 μg, or about 180 μg, of drug per dose of PEGASYS®, subcutaneously qw, qow, three times per month, or monthly (v) PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly or (vi) mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of INFERGEN® containing an amount of about 1 μg to about 30 μg of drug per dose of INFERGEN®, subcutaneously qd, qod, tiw, biw, qw, qow, three times per month, once monthly, or per day continuously or substantially continuously, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGylated consensus IFN-α (PEG-CIFN) containing an amount of about 10 μg to about 100 μg, or about 45 μg to about 60 μg, of CIFN amino acid weight per dose of PEG-CIFN subcutaneously qw, qow, three times per month, or monthly, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of IFN-α2a, 2b or 2c containing an amount of about 3 MU to about 10 MU of drug per dose of IFN-α2a, 2b or 2c, subcutaneously qd, qod, tiw, biw, or per day continuously or substantially continuously, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGASYS® containing an amount of about 90 μg to about 360 μg, or about 180 μg, of drug per dose of PEGASYS®, subcutaneously qw, qow, three times per month, or monthly, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of an IFN-α selected from (i) INFERGEN® containing an amount of about 1 μg to about 30 μg of drug per dose of INFERGEN®, subcutaneously qd, qod, tiw, biw, qw, qow, three times per month, once monthly, or per day continuously or substantially continuously (ii) PEGylated consensus IFN-α (PEG-CIFN) containing an amount of about 10 μg to about 100 μg, or about 45 μg to about 60 μg, of CIFN amino acid weight per dose of PEG-CIFN subcutaneously qw, qow, three times per month, or monthly (iii) IFN-α2a, 2b or 2c containing an amount of about 3 MU to about 10 MU of drug per dose of IFN-α2a, 2b or 2c subcutaneously qd, qod, tiw, biw, or per day continuously or substantially continuously (iv) PEGASYS® containing an amount of about 90 μg to about 360 μg, or about 180 μg, of drug per dose of PEGASYS®, subcutaneously qw, qow, three times per month, or monthly (v) PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly or (vi) mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of INFERGEN® containing an amount of about 1 μg to about 30 μg of drug per dose of INFERGEN®, subcutaneously qd, qod, tiw, biw, qw, qow, three times per month, once monthly, or per day continuously or substantially continuously, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGylated consensus IFN-α (PEG-CIFN) containing an amount of about 10 μg to about 100 μg, or about 45 μg to about 60 μg, of CIFN amino acid weight per dose of PEG-CIFN, subcutaneously qw, qow, three times per month, or monthly for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of IFN-α2a, 2b or 2c containing an amount of about 3 MU to about 10 MU of drug per dose of IFN-α2a, 2b or 2c, subcutaneously qd, qod, tiw, biw, or per day continuously or substantially continuously, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; c) a dosage of an HCV NS5B RNA-dependent RNA polymerase inhibitor containing an amount of from about 0.01 mg to about 100 mg of drug per ldlogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGASYS® containing an amount of about 90 μg to about 360 μg, or about 180 μg, of drug per dose of PEGASYS®, subcutaneously qw, qow, three times per month, or monthly for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

Any of the above-described regimens involving administration of IFN-α, pirfenidone or a pirfenidone analog, and an NS3 protease inhibitor; IFN-α, pirfenidone or a pirfenidone analog, and an NS5B RNA-dependent RNA polymerase inhibitor; or IFN-α, pirfenidone or a pirfenidone analog, an NS3 protease inhibitor and an NS5B RNA-dependent RNA polymerase inhibitor, can be modified to further comprise administering an effective amount of an additional immunomodulatory agent. Thus, in some embodiments, a subject combination therapy method for treating an HCV infection in an individual comprises co-administering effective amounts of IFN-α, pirfenidone or a pirfenidone analog, an NS3 protease inhibitor, and a second immunomodulatory agent. In other embodiments, a subject combination therapy method for treating an HCV infection in an individual comprises co-administering effective amounts of IFN-α, pirfenidone or a pirfenidone analog, an NS5B RNA-dependent RNA polymerase inhibitor, and a second immunomodulatory agent. In other embodiments, a subject combination therapy method for treating an HCV infection in an individual comprises co-administering effective amounts of IFN-α, pirfenidone or a pirfenidone analog, an NS3 protease inhibitor, an NS5B RNA-dependent RNA polymerase inhibitor, and a second immunomodulatory agent.

As non-limiting examples, any of the above-described IFN-α, pirfenidone or pirfenidone analog, and HCV enzyme inhibitor combination regimens can be modified to include: (a) administering a dosage of IFN-γ containing an amount of from about 10 μg to about 300 μg of drug per dose of IFN-γ, subcutaneously qd, qod, tiw, or biw, or per day substantially continuously or continuously; (b) administering a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; (c) a dosage of Zadaxin™ containing an amount of 1.0 mg or 1.6 mg, administered subcutaneously twice per week for the desired treatment duration; (d) a dosage of ribavirin or a derivative thereof, in an amount of about 400 mg, 800 mg, 1000 mg, or 1200 mg orally daily for the desired treatment duration; (e) a dosage of levovirin, in an amount of about 400 mg, 600 mg, 800 mg, 1000 mg, or 1200 mg orally daily for the desired treatment duration; or (f) a dosage of Viramidine™ in an amount of from about 800 mg to about 1600 mg orally daily for the desired treatment duration.

IFN-α, Thymosin-α, and an HCV Enzyme Inhibitor in Combination Therapy

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of an IFN-α selected from (i) INFERGEN® containing an amount of about 1 μg to about 30 μg of drug per dose of INFERGEN®, subcutaneously qd, qod, tiw, biw, qw, qow, three times per month, once monthly, or per day continuously or substantially continuously (ii) PEGylated consensus IFN-α (PEG-CIFN) containing an amount of about 10 μg to about 1000 μg, or about 45 μg to about 60 μg, of CIFN amino acid weight per dose of PEG-CIFN subcutaneously qw, qow, three times per month, or monthly (iii) IFN-α2a, 2b or 2c containing an amount of about 3 MU to about 10 MU of drug per dose of IFN-α2a, 2b or 2c subcutaneously qd, qod, tiw, biw, or per day continuously or substantially continuously (iv) PEGASYS® containing an amount of about 90 μg to about 360 μg, or about 180 μg, of drug per dose of PEGASYS®, subcutaneously qw, qow, three times per month, or monthly (v) PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly or (vi) mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of Zadaxin™ containing an amount of 1.0 mg or 1.6 mg, administered subcutaneously twice per week for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of INFERGEN® containing an amount of about 1 μg to about 30 μg of drug per dose of INFERGEN®, subcutaneously qd, qod, tiw, biw, qw, qow, three times per month, once monthly, or per day continuously or substantially continuously, for the desired treatment duration; b) a dosage of Zadaxin™ containing an amount of 1.0 mg or 1.6 mg, administered subcutaneously twice per week for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGylated consensus IFN-α (PEG-CIFN) containing an amount of about 10 μg to about 100 μg, or about 45 μg to about 60 μg, of CIFN amino acid weight per dose of PEG-CIFN, subcutaneously qw, qow, three times per month, or monthly, for the desired treatment duration; b) a dosage of Zadaxin™ containing an amount of 1.0 mg or 1.6 mg, administered subcutaneously twice per week for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of IFN-α2a, 2b or 2c containing an amount of about 3 MU to about 10 MU of drug per dose of IFN-α2a, 2b or 2c, subcutaneously qd, qod, tiw, biw, or per day continuously or substantially continuously, for the desired treatment duration; b) a dosage of Zadaxin™ containing an amount of 1.0 mg or 1.6 mg, administered subcutaneously twice per week for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGASYS® containing an amount of about 90 μg to about 360 μg, or about 180 μg, of drug per dose of PEGASYS®, subcutaneously qw, qow, three times per month, or monthly, for the desired treatment duration; b) a dosage of Zadaxin™ containing an amount of 1.0 mg or 1.6 mg, administered subcutaneously twice per week for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly, for the desired treatment duration; b) a dosage of Zadaxin™ containing an amount of 1.0 mg or 1.6 mg, administered subcutaneously twice per week for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of Zadaxin™ containing an amount of 1.0 mg or 1.6 mg, administered subcutaneously twice per week for the desired treatment duration; and c) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of an IFN-α selected from (i) INFERGEN® containing an amount of about 1 μg to about 30 μg of drug per dose of INFERGEN®, subcutaneously qd, qod, tiw, biw, qw, qow, three times per month, once monthly, or per day continuously or substantially continuously (ii) PEGylated consensus IFN-α (PEG-CIFN) containing an amount of about 10 μg to about 100 μg, or about 45 μg to about 60 μg, of CIFN amino acid weight per dose of PEG-CIFN subcutaneously qw, qow, three times per month, or monthly (iii) IFN-α2a, 2b or 2c containing an amount of about 3 MU to about 10 MU of drug per dose of IFN-α2a, 2b or 2c subcutaneously qd, qod, tiw, biw, or per day continuously or substantially continuously (iv) PEGASYS® containing an amount of about 90 μg to about 360 μg, or about 180 μg, of drug per dose of PEGASYS®, subcutaneously qw, qow, three times per month, or monthly (v) PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly or (vi) mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of Zadaxin™ containing an amount of 1.0 mg or 1.6 mg, administered subcutaneously twice per week for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of INFERGEN® containing an amount of about 1 μg to about 30 μg of drug per dose of INFERGEN®, subcutaneously qd, qod, tiw, biw, qw, qow, three times per month, once monthly, or per day continuously or substantially continuously, for the desired treatment duration; b) a dosage of Zadaxin™ containing an amount of 1.0 mg or 1.6 mg, administered subcutaneously twice per week for the desired treatment duration; and c) a dosage of an HCV NS5B RNA-dependent RNA polymerase inhibitor containing an amount of from about 0.0 1 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGylated consensus IFN-α (PEG-CIFN) containing an amount of about 10 μg to about 100 μg, or about 45 μg to about 60 μg, of CIFN amino acid weight per dose of PEG-CIFN, subcutaneously qw, qow, three times per month, or monthly, for the desired treatment duration; b) a dosage of Zadaxin™ containing an amount of 1.0 mg or 1.6 mg, administered subcutaneously twice per week for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of IFN-α2a, 2b or 2c containing an amount of about 3 MU to about 10 MU of drug per dose of IFN-α2a, 2b or 2c, subcutaneously qd, qod, tiw, biw, or per day continuously or substantially continuously, for the desired treatment duration; b) a dosage of Zadaxin™ containing an amount of 1.0 mg or 1.6 mg, administered subcutaneously twice per week for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGASYS® containing an amount of about 90 μg to about 360 μg, or about 180 μg, of drug per dose of PEGASYS®, subcutaneously qw, qow, three times per month, or monthly, for the desired treatment duration; b) a dosage of Zadaxin™ containing an amount of 1.0 mg or 1.6 mg, administered subcutaneously twice per week for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly, for the desired treatment duration; b) a dosage of Zadaxin™ containing an amount of 1.0 mg or 1.6 mg, administered subcutaneously twice per week for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of Zadaxin™ containing an amount of 1.0 mg or 1.6 mg, administered subcutaneously twice per week for the desired treatment duration; and c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of an IFN-α selected from (i) INFERGEN® containing an amount of about 1 μg to about 30 μg of drug per dose of INFERGEN®, subcutaneously qd, qod, tiw, biw, qw, qow, three times per month, once monthly, or per day continuously or substantially continuously (ii) PEGylated consensus IFN-α (PEG-CIFN) containing an amount of about 10 μg to about 100 μg, or about 45 μg to about 60 μg, of CIFN amino acid weight per dose of PEG-CIFN subcutaneously qw, qow, three times per month, or monthly (iii) IFN-α2a, 2b or 2c containing an amount of about 3 MU to about 10 MU of drug per dose of IFN-α2a, 2b or 2c subcutaneously qd, qod, tiw, biw, or per day continuously or substantially continuously (iv) PEGASYS® containing an amount of about 90 μg to about 360 μg, or about 180 μg, of drug per dose of PEGASYS®, subcutaneously qw, qow, three times per month, or monthly (v) PEG-INTRON® containing an amount of about 0.75 μg to about 3.0 μg, or about 1.0 μg, of drug per kilogram of body weight per dose of PEG-INTRON®, subcutaneously biw, qw, qow, three times per month, or monthly or (vi) mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every . 14 days, three times per month, or monthly, for the desired treatment duration; b) a dosage of Zadaxin™ containing an amount of 1.0 mg or 1.6 mg, administered subcutaneously twice per week for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.0 1 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of INFERGEN® containing an amount of about 1 μg to about 30 μg of drug per dose of INFERGEN®, subcutaneously qd, qod, tiw, biw, qw, qow, three times per month, once monthly, or per day continuously or substantially continuously, for the desired treatment duration; b) a dosage of Zadaxin™ containing an amount of 1.0 mg or 1.6 mg, administered subcutaneously twice per week for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGylated consensus IFN-α (PEG-CIFN) containing an amount of about 10 μg to about 100 μg, or about 45 μg to about 60 μg, of CIFN amino acid weight per dose of PEG-CIFN, subcutaneously qw, qow, three times per month, or monthly, for the desired treatment duration; b) a dosage of Zadaxin™ containing an amount of 1.0 mg or 1.6 mg, administered subcutaneously twice per week for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per ldlogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of IFN-α2a, 2b or 2c containing an amount of about 3 MU to about 10 MU of drug per dose of IFN-α2a, 2b or 2c, subcutaneously qd, qod, tiw, biw, or per day continuously or substantially continuously, for the desired treatment duration; b) a dosage of Zadaxin™ containing an amount of 1.0 mg or 1.6 mg, administered subcutaneously twice per week for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEGASYS® containing an amount of about 90 μg to about 360 μg, or about 180 μg, of drug per dose of PEGASYS®, subcutaneously qw, qow, three times per month, or monthly for the desired treatment duration; b) a dosage of Zadaxin™ containing an amount of 1.0 mg or 1.6 mg, administered subcutaneously twice per week for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of PEG-INTRON® containing an amount of about 0.75 μg to about 3.0 μg, or about 1.0 μg, of drug per kilogram of body weight per dose of PEG-INTRON® subcutaneously biw, qw, qow, three times per month, or monthly, for the desired treatment duration; b) a dosage of Zadaxin™ containing an amount of 1.0 mg or 1.6 mg, administered subcutaneously twice per week for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

In some embodiments, the invention provides a combination therapy method using combined effective amounts of i) a Type I interferon receptor agonist, ii) an immunomodulatory agent; and iii) an HCV enzyme inhibitor in the treatment of an HCV infection in a patient, comprising co-administering to the patient a) a dosage of mono PEG(30 kD, linear)-ylated consensus IFN-α containing an amount of from about 100 μg to about 200 μg, or about 150 μg, of drug per dose of mono PEG(30 kD, linear)-ylated consensus IFN-α, subcutaneously qw, qow, once every 8 days to once every 14 days, three times per month, or monthly, for the desired treatment duration; b) a dosage of Zadaxin™ containing an amount of 1.0 mg or 1.6 mg, administered subcutaneously twice per week for the desired treatment duration; c) a dosage of an HCV NS5B RNA-dependent RNA polymerase inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration; and d) a dosage of an HCV NS3 protease inhibitor containing an amount of from about 0.01 mg to about 100 mg of drug per kilogram of body weight per dose, orally tid, bid, qd, qod, tiw, biw, qw, qow, three times per month, or once monthly, for the desired treatment duration, to achieve a sustained viral response.

Any of the above-described regimens involving administration of: (1) IFN-α, thymosin-α, and an NS3 protease inhibitor; (2) IFN-α, thymosin-α, and an NS5B RNA-dependent RNA polymerase inhibitor; or (3) IFN-α, thymosin-α, an NS3 protease inhibitor and an NS5B RNA-dependent RNA polymerase inhibitor; can be modified to further comprise administering an effective amount of an additional antiviral agent. Thus, in some embodiments, a subject combination therapy method for treating an HCV infection in an individual comprises co-administering effective amounts of IFN-α, thymosin-α, an NS3 protease inhibitor, and a second immunomodulatory agent. In other embodiments, a subject combination therapy method for treating an HCV infection in an individual comprises co-administering effective amounts of IFN-α, thymosin-α, an NS5B RNA-dependent RNA polymerase inhibitor, and a second immunomodulatory agent. In other embodiments, a subject combination therapy method for treating an HCV infection in an individual comprises co-administering effective amounts of IFN-α, thymosin-α, an NS3 protease inhibitor, an NS5B RNA-dependent RNA polymerase inhibitor, and a second immunomodulatory agent.

As non-limiting examples, any of the above-described IFN-α, thymosin-α, and HCV enzyme inhibitor combination regimens can be modified to include: (a) administering a dosage of IFN-γ containing an amount of from about 10 μg to about 300 μg of drug per dose of IFN-γ, subcutaneously qd, qod, tiw, or biw, or per day substantially continuously or continuously; (b) administering a dosage of a TNF-α antagonist selected from (i) ENBREL® in an amount of about 25 mg of drug subcutaneously biw (ii) REMICADE® in an amount of about 3 mg/kg to about 10 mg/kg of drug intravenously qw, qow, three times per month, once monthly, once every 6 weeks, or once every 8 weeks or (iii) HUMIRA™ in an amount of about 40 mg of drug subcutaneously qw, qow, three times per month, once monthly, once every 6 weeks, or once every 8 weeks, for the desired treatment duration; (c) administering a dosage of pirfenidone or a pirfenidone analog, in a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally for the desired treatment duration; (d) administering a dosage of ribavirin or a derivative thereof, in an amount of about 400 mg, 800 mg, 1000 mg, or 1200 mg orally daily for the desired treatment duration; (e) administering a dosage of levovirin, in an amount of about 400 mg, 600 mg, 800 mg, 1000 mg, or 1200 mg orally daily for the desired treatment duration; or (f) administering a dosage of viramidine in an amount of from about 800 mg to about 1600 mg orally daily for the desired treatment duration.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 100 μg of drug per dose, subcutaneously once weekly or once every 8 days for the desired treatment duration.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 150 μg of drug per dose, subcutaneously once weekly or once every 8 days for the desired treatment duration.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 200 μg of drug per dose, subcutaneously once weekly or once every 8 days for the desired treatment duration.

As non-limiting examples, any of the above-described methods featuring an IFN-α regimen can be modified to replace the subject IFN-α regimen with a regimen of INFERGEN® interferon alfacon-1 comprising administering a dosage of INFERGEN® interferon alfacon-1 containing an amount of 9 μg of drug per dose, subcutaneously once daily or three times per week for the desired treatment duration.

As non-limiting examples, any of the above-described methods featuring an IFN-α regimen can be modified to replace the subject IFN-α regimen with a regimen of INFERGEN® interferon alfacon-1 comprising administering a dosage of INFERGEN® interferon alfacon-1 containing an amount of 15 μg of drug per dose, subcutaneously once daily or three times per week for the desired treatment duration.

As non-limiting examples, any of the above-described methods featuring an IFN-γ regimen can be modified to replace the subject IFN-γ regimen with a regimen of IFN-γ comprising administering a dosage of IFN-γ containing an amount of 25 μg of drug per dose, subcutaneously three times per week for the desired treatment duration.

As non-limiting examples, any of the above-described methods featuring an IFN-γ regimen can be modified to replace the subject IFN-γ regimen with a regimen of IFN-γ comprising administering a dosage of IFN-γ containing an amount of 50 μg of drug per dose, subcutaneously three times per week for the desired treatment duration.

As non-limiting examples, any of the above-described methods featuring an IFN-γ regimen can be modified to replace the subject IFN-γ regimen with a regimen of IFN-γ comprising administering a dosage of IFN-γ containing an amount of 100 μg of drug per dose, subcutaneously three times per week for the desired treatment duration.

As non-limiting examples, any of the above-described methods featuring an IFN-α and IFN-γ combination regimen can be modified to replace the subject IFN-α and IFN-γcombination regimen with an IFN-α and IFN-γ combination regimen comprising: (a) administering a dosage of monoPEG (30 kD, linear)-ylated consensus IFN-α containing an amount of 100 μg of drug per dose, subcutaneously once weekly or once every 8 days; and (b) administering a dosage of IFN-γ containing an amount of 50 μg of drug per dose, subcutaneously three times per week; for the desired treatment duration.

As non-limiting examples, any of the above-described methods featuring a TNF antagonist regimen can be modified to replace the subject TNF antagonist regimen with a TNF antagonist regimen comprising administering a dosage of a TNF antagonist selected from the group of: (a) etanercept in an amount of 25 mg of drug per dose subcutaneously twice per week, (b) infliximab in an amount of 3 mg of drug per kilogram of body weight per dose intravenously at weeks 0, 2 and 6, and every 8 weeks thereafter, or (c) adalimumab in an amount of 40 mg of drug per dose subcutaneously once weekly or once every 2 weeks; for the desired treatment duration.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 100 μg of drug per dose, subcutaneously once weekly or once every 8 days; and (b) administering a dosage of IFN-γ containing an amount of 100 μg of drug per dose, subcutaneously three times per week; for the desired treatment duration.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 150 μg of drug per dose, subcutaneously once weekly or once every 8 days; and (b) administering a dosage of IFN-γ containing an amount of 50 μg of drug per dose, subcutaneously three times per week; for the desired treatment duration.

As non-limiting examples, any of the above-described methods featuring an IFN-α and IFN-γ, combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 150 μg of drug per dose, subcutaneously once weekly or once every 8 days; and (b) administering a dosage of IFN-γ containing an amount of 100 μg of drug per dose, subcutaneously three times per week; for the desired treatment duration.

As non-limiting examples, any of the above-described methods featuring an IFN-α and IFN-γ combination regimen can be modified to replace the subject IFN-α and IFN-γcombination regimen with an IFN-α and IFN-γ combination regimen comprising: (a) administering a dosage of monoPEG (30 kD, linear)-ylated consensus IFN-α containing an amount of 200 μg of drug per dose, subcutaneously once weekly or once every 8 days; and (b) administering a dosage of IFN-γ containing an amount of 50 μg of drug per dose, subcutaneously three times per week; for the desired treatment duration.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 200 μg of drug per dose, subcutaneously once weekly or once every 8 days; and (b) administering a dosage of IFN-γ containing an amount of 100 μg of drug per dose, subcutaneously three times per week; for the desired treatment duration.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 25 μg of drug per dose, subcutaneously three times per week; for the desired treatment duration.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 50 μg of drug per dose, subcutaneously three times per week; for the desired treatment duration.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 100 μg of drug per dose, subcutaneously three times per week; for the desired treatment duration.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 25 μg of drug per dose, subcutaneously three times per week; for the desired treatment duration.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 50 μg of drug per dose, subcutaneously three times per week; for the desired treatment duration.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 100 μg of drug per dose, subcutaneously three times per week; for the desired treatment duration.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 25 μg of drug per dose, subcutaneously three times per week; for the desired treatment duration.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 50 μg of drug per dose, subcutaneously three times per week; for the desired treatment duration.

As non-limiting examples, any of the above-described methods featuring an IFN-α, and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 100 μg of drug per dose, subcutaneously three times per week; for the desired treatment duration.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously once daily; and (b) administering a dosage of IFN-γ containing an amount of 25 μg of drug per dose, subcutaneously three times per week; for the desired treatment duration.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN(M interferon alfacon-1 containing an amount of 15 μg of drug per dose, subcutaneously once daily; and (b) administering a dosage of IFN-γ containing an amount of 50 μg of drug per dose, subcutaneously three times per week; for the desired treatment duration.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously once daily; and (b) administering a dosage of IFN-γ containing an amount of 100 μg of drug per dose, subcutaneously three times per week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 100 μg of drug per dose, subcutaneously once weekly or once every 8 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 100 μg of drug per dose, subcutaneously once weekly or once every 8 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 150 μg of drug per dose, subcutaneously once weekly or once every 8 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 150 μg of drug per dose, subcutaneously once weekly or once every 8 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 200 μg of drug per dose, subcutaneously once weekly or once every 8 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 200 μg of drug per dose, subcutaneously once weekly or once every 8 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) ilifliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-αcontaining an amount of 100 μg of drug per dose, subcutaneously once weekly or once every 8 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-α containing an amount of 150 μg of drug per dose, subcutaneously once weekly or once every 8 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-α containing an amount of 200 μg of drug per dose, subcutaneously once weekly or once every 8 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of INFERGEN(I interferon alfacon-1 containing an amount of 9 μg of drug per dose, subcutaneously once daily or three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of INFERGEN® interferon alfacon-1 containing an amount of 15 μg of drug per dose, subcutaneously once daily or three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 25 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 50 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 100 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration.

As non-limiting examples, any of the above-described methods that includes a regimen of monoPEG (30 kD, linear)-ylated consensus IFN-α can be modified to replace the regimen of monoPEG (30 kD, linear)-ylated consensus IFN-α with a regimen of peginterferon alfa-2a comprising administering a dosage of peginterferon alfa-2a containing an amount of 180 μg of drug per dose, subcutaneously once weekly for the desired treatment duration.

As non-limiting examples, any of the above-described methods that includes a regimen of monoPEG (30 kD, linear)-ylated consensus IFN-α can be modified to replace the regimen of monoPEG (30 kD, linear)-ylated consensus IFN-α with a regimen of peginterferon alfa-2b comprising administering a dosage of peginterferon alfa-2b containing an amount of 1.0 μg of drug per kilogram of body weight per dose, subcutaneously once or twice weekly for the desired treatment duration.

As non-limiting examples, any of the above-described methods can be modified to include administering a dosage of ribavirin containing an amount of 400 mg, 800 mg, 1000 mg or 1200 mg of drug orally per day, optionally in two or more divided doses per day, for the desired treatment duration.

As non-limiting examples, any of the above-described methods can be modified to include administering a dosage of ribavirin containing (i) an amount of 1000 mg of drug orally per day for patients having a body weight of less than 75 kg or (ii) an amount of 1200 mg of drug orally per day for patients having a body weight of greater than or equal to 75 kg, optionally in two or more divided doses per day, for the desired treatment duration.

As non-limiting examples, any of the above-described methods featuring an HCV NS3 inhibitor regimen can be modified to replace the subject HCV NS3 inhibitor regimen with an HCV NS3 inhibitor regimen comprising administering a dosage of 0.01 mg to 0.1 mg of drug per kilogram of body weight orally daily, optionally in two or more divided doses per day, for the desired treatment duration.

As non-limiting examples, any of the above-described methods featuring an HCV NS3 inhibitor regimen can be modified to replace the subject HCV NS3 inhibitor regimen with an HCV NS3 inhibitor regimen comprising administering a dosage of 0.1 mg to 1 mg of drug per kilogram of body weight orally daily, optionally in two or more divided doses per day, for the desired treatment duration.

As non-limiting examples, any of the above-described methods featuring an HCV NS3 inhibitor regimen can be modified to replace the subject HCV NS3 inhibitor regimen with an HCV NS3 inhibitor regimen comprising administering a dosage of 1 mg to 10 mg of drug per kilogram of body weight orally daily, optionally in two or more divided doses per day, for the desired treatment duration.

As non-limiting examples, any of the above-described methods featuring an HCV NS3 inhibitor regimen can be modified to replace the subject HCV NS3 inhibitor regimen with an HCV NS3 inhibitor regimen comprising administering a dosage of 10 mg to 100 mg of drug per kilogram of body weight orally daily, optionally in two or more divided doses per day, for the desired treatment duration.

As non-limiting examples, any of the above-described methods featuring an HCV NS5B inhibitor regimen can be modified to replace the subject HCV NS5B inhibitor regimen with an HCV NS5B inhibitor regimen comprising administering a dosage of 0.01 mg to 0.1 mg of drug per kilogram of body weight orally daily, optionally in two or more divided doses per day, for the desired treatment duration.

As non-limiting examples, any of the above-described methods featuring an HCV NS5B inhibitor regimen can be modified to replace the subject HCV NS5B inhibitor regimen with an HCV NS5B inhibitor regimen comprising administering a dosage of 0.1 mg to 1 mg of drug per kilogram of body weight orally daily, optionally in two or more divided doses per day, for the desired treatment duration.

As non-limiting examples, any of the above-described methods featuring an HCV NS5B inhibitor regimen can be modified to replace the subject HCV NS5B inhibitor regimen with an HCV NS5B inhibitor regimen comprising administering a dosage of 1 mg to 10 mg of drug per kilogram of body weight orally daily, optionally in two or more divided doses per day, for the desired treatment duration.

As non-limiting examples, any of the above-described methods featuring an HCV NS5B inhibitor regimen can be modified to replace the subject HCV NS5B inhibitor regimen with an HCV NS5B inhibitor regimen comprising administering a dosage of 10 mg to 100 mg of drug per kilogram of body weight orally daily, optionally in two or more divided doses per day, for the desired treatment duration.

Patient Identification

In certain embodiments, the specific regimen of drug therapy used in treatment of the HCV patient is selected according to certain disease parameters exhibited by the patient, such as the initial viral load, genotype of the HCV infection in the patient, liver histology and/or stage of liver fibrosis in the patient.

Thus, in some embodiments, the present invention provides any of the above-described methods for the treatment of HCV infection in which the subject method is modified to treat a treatment failure patient for a duration of 48 weeks.

In other embodiments, the invention provides any of the above-described methods for HCV in which the subject method is modified to treat a non-responder patient, where the patient receives a 48 week course of therapy.

In other embodiments, the invention provides any of the above-described methods for the treatment of HCV infection in which the subject method is modified to treat a relapser patient, where the patient receives a 48 week course of therapy.

In other embodiments, the invention provides any of the above-described methods for the treatment of HCV infection in which the subject method is modified to treat a naive patient infected with HCV genotype 1, where the patient receives a 48 week course of therapy.

In other embodiments, the invention provides any of the above-described methods for the treatment of HCV infection in which the subject method is modified to treat a naive patient infected with HCV genotype 4, where the patient receives a 48 week course of therapy.

In other embodiments, the invention provides any of the above-described methods for the treatment of HCV infection in which the subject method is modified to treat a naive patient infected with HCV genotype 1, where the patient has a high viral load (HVL), where “HVL” refers to an HCV viral load of greater than 2×10⁶ HCV genome copies per mL serum, and where the patient receives a 48 week course of therapy.

In one embodiment, the invention provides any of the above-described methods for the treatment of an HCV infection, where the subject method is modified to include the steps of (1) identifying a patient having advanced or severe stage liver fibrosis as measured by a Knodell score of 3 or 4 and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides any of the above-described methods for the treatment of an HCV infection, where the subject method is modified to include the steps of (1) identifying a patient having advanced or severe stage liver fibrosis as measured by a Knodell score of 3 or 4 and then (2) administering to the patient the drug therapy of the subject method for a time period of about 40 weeks to about 50 weeks, or about 48 weeks.

In another embodiment, the invention provides any of the above-described methods for the treatment of an HCV infection, where the subject method is modified to include the steps of (1) identifying a patient having an HCV genotype 1 infection and an initial viral load of greater than 2 million viral genome copies per ml of patient serum and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides any of the above-described methods for the treatment of an HCV infection, where the subject method is modified to include the steps of (1) identifying a patient having an HCV genotype 1 infection and an initial viral load of greater than 2 million viral genome copies per ml of patient serum and then (2) administering to the patient the drug therapy of the subject method for a time period of about 40 weeks to about 50 weeks, or about 48 weeks.

In another embodiment, the invention provides any of the above-described methods for the treatment of an HCV infection, where the subject method is modified to include the steps of (1) identifying a patient having an HCV genotype 1 infection and an initial viral load of greater than 2 million viral genome copies per ml of patient serum and no or early stage liver fibrosis as measured by a Knodell score of 0, 1, or 2 and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides any of the above-described methods for the treatment of an HCV infection, where the subject method is modified to include the steps of (1) identifying a patient having an HCV genotype 1 infection and an initial viral load of greater than 2 million viral genome copies per ml of patient serum and no or early stage liver fibrosis as measured by a Knodell score of 0, 1, or 2 and then (2) administering to the patient the drug therapy of the subject method for a time period of about 40 weeks to about 50 weeks, or about 48 weeks.

In another embodiment, the invention provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

In another embodiment, the invention provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 20 weeks to about 24 weeks.

In another embodiment, the invention provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 24 weeks to about 48 weeks.

In another embodiment, the invention provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

In another embodiment, the invention provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 20 weeks to about 24 weeks.

In another embodiment, the invention provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of at least about 24 weeks.

In another embodiment, the invention provides any of the above-described methods for the treatment of an HCV infection, where the subject method is modified to include the steps of (1) identifying a patient having an HCV genotype 1 or 4 infection and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides any of the above-described methods for the treatment of an HCV infection, where the subject method is modified to include the steps of (1) identifying a patient having an HCV infection characterized by any of HCV genotypes 5, 6, 7, 8 and 9 and then (2) administering to the patient the drug therapy of the subject method for a time period of about 20 weeks to about 50 weeks.

In another embodiment, the invention provides any of the above-described methods for the treatment of an HCV infection, where the subject method is modified to include the steps of (1) identifying a patient having an HCV infection characterized by any of HCV genotypes 5, 6, 7, 8 and 9 and then (2) administering to the patient the drug therapy of the subject method for a time period of at least about 24 weeks and up to about 48 weeks.

Subjects Suitable for Treatment

Individuals who are to be treated according to the methods of the invention include individuals who have been clinically diagnosed as infected with HCV. Individuals who are infected with HCV are identified as having HCV RNA in their blood, and/or having anti-HCV antibody in their serum.

Individuals who are clinically diagnosed as infected with HCV include naive individuals (e.g., individuals not previously treated for HCV, particularly those who have not previously received IFN-α-based and/or ribavirin-based therapy) and individuals who have failed prior treatment for HCV (“treatment failure” patients). Treatment failure patients include non-responders (i.e., individuals in whom the HCV titer was not significantly or sufficiently reduced by a previous treatment for HCV, e.g., a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-α monotherapy, a previous IFN-α and ribavirin combination therapy, or a previous pegylated IFN-α and ribavirin combination therapy, whose HCV titer decreased, and subsequently increased).

In particular embodiments of interest, individuals have an HCV titer of at least about 105, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

Also of interest are HCV-positive individuals (as described above) who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In particular embodiments of interest, HCV-positive individuals with stage 3 or 4 liver fibrosis according to the METAVIR scoring system are suitable for treatment with the methods of the present invention. In other embodiments, individuals suitable for treatment with the methods of the instant invention are patients with decompensated cirrhosis with clinical manifestations, including patients with far-advanced liver cirrhosis, including those awaiting liver transplantation. In still other embodiments, individuals suitable for treatment with the methods of the instant invention include patients with milder degrees of fibrosis including those with early fibrosis (stages 1 and 2 in the METAVIR, Ludwig, and Scheuer scoring systems; or stages 1, 2, or 3 in the Ishak scoring system.).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for treating a hepatitis C virus infection in an individual, the method comprising administering to the individual a IFN-α, at least one immunomodulatory agent, and at least one inhibitor of an HCV enzyme, in amounts effective to achieve a sustained viral response, wherein the immunomodulatory agent is one or more of IFN-γ, pirfenidone or a pirfenidone analog, a TNF antagonist, and thymosin-α.

2. The method of claim 1, wherein the inhibitor of an HCV enzyme is an HCV NS3 protease inhibitor.

3. The method of claim 1, wherein the inhibitor of an HCV enzyme is an HCV NS5B RNA-dependent RNA polymerase inhibitor.

4. The method of claim 1, comprising administering an HCV NS3 protease inhibitor and an HCV NS5B RNA-dependent RNA polymerase inhibitor.

5. The method of claim 1, wherein the immunomodulatory agent is IFN-γ administered subcutaneously in an amount of from about 10 μg to about 300 μg.

6. The method of claim 1, wherein the immunomodulatory agent is pirfenidone or a pirfenidone analog administered orally daily in an amount of from about 400 mg to about 3600 mg.

7. The method of claim 1, wherein the immunomodulatory agent is a TNF antagonist.

8. The method of claim 7, wherein the TNF antagonist is selected from the group consisting of etanercept, infliximab, and adalimumab.

9. The method of claim 1, wherein the immunomodulatory agent is thymosin-α administered subcutaneously twice weekly in an amount of from about 1.0 mg to about 1.6 mg.

10. The method of any of claims 1-9, wherein the method further comprises administering to the individual an effective amount of a nucleoside analog.

11. The method of claim 10, wherein the nucleoside analog is ribavirin.

12. The method of claim 10, wherein the nucleoside analog is levovirin.

13. The method of claim 10, wherein the nucleoside analog is viramidine.

14. The method of claim 10, wherein the nucleoside analog is isatoribine.

15. The method of claim 10, wherein the nucleoside analog is an L-nucleoside.

16. The method of claim 1, wherein the IFN-α is monoPEG (30 kD, linear)-ylated consensus IFN-α administered at a dosing interval of every 8 days to every 14 days.

17. The method of claim 1, wherein the IFN-α is monoPEG (30 1kD, linear)-ylated consensus IFN-α administered at a dosing interval of once every 7 days.

18. The method of any of claims 1-15, wherein the IFN-α is a pegylated IFN-α.

19. The method of claim 18, wherein the pegylated IFN-α is a pegylated IFN-α2 or a pegylated consensus IFN-α.

20. The method of claim 19, wherein the pegylated IFN-α is selected from the group consisting of peginterferon alfa-2a, peginterferon alfa-2b, and monoPEG (30 kD, linear)-ylated consensus IFN-α.

21. The method of claim 1, comprising administering effective amounts of IFN-γ and pirfenidone or a pirfenidone analog.

22. The method of claim 1, comprising administering effective amounts of IFN-γ and thymosin-α.

23. The method of claim 1, comprising administering effective amounts of IFN-γ and ribavirin.

24. The method of claim 1, comprising administering effective amounts of IFN-γ and levovirin.

25. The method of claim 1, comprising administering effective amounts of IFN-γ and viramidine.

26. The method of claim 1, comprising administering effective amounts of IFN-γ and an L-nucleoside.

27. The method of claim 1, comprising administering effective amounts of a TNF antagonist and IFN-γ.

28. The method of claim 27, wherein the TNF antagonist is selected from the group consisting of etanercept, infliximab and adalimumab.

29. The method of any of claims 21, 22, 27 or 28, wherein the method further comprises administering to the individual an effective amount of a nucleoside analog.

30. The method of claim 29, wherein the nucleoside analog is ribavirin.

31. The method of claim 29, wherein the nucleoside analog is levovirin.

32. The method of claim 29, wherein the nucleoside analog is viramidine.

33. The method of claim 29, wherein the nucleoside analog is an L-nucleoside.

34. The method of any of claims 21-33, wherein the IFN-α is a pegylated IFN-α.

35. The method of claim 34, wherein the pegylated IFN-α is a pegylated IFN-α2 or a pegylated consensus IFN-α.

36. The method of claim 34, wherein the pegylated IFN-α is selected from the group consisting of peginterferon alfa-2a, peginterferon alfa-2b and monoPEG (30 kD, linear)-ylated consensus IFN-α.

37. The method of any of claims 5-36, wherein the HCV enzyme inhibitor is an HCV NS3 protease inhibitor.

38. The method of any of claims 5-36, wherein the HCV enzyme inhibitor is an HCV NS5B RNA-dependent RNA polymerase inhibitor.
